(19) United States
(12) Patent Application Publication
(10) Pub. No.: US 2026/0199445 A1
(43) Pub. Date: Jul. 16, 2026

(54) VACCINE AGAINST KLEBSIELLA PNEUMONIAE

(71) Applicant: Idorsia Pharmaceuticals Ltd, Allschwil (CH)

(72) Inventors: Felix BROECKER, Berlin (DE); Sharavathi Guddehalli PARAMESWARAPPA, Berlin (DE); Claney Lebev PEREIRA, Berlin (DE); Marilda P. LISBOA, Berlin (DE); Arne VON BONIN, Allschwil (CH)

(21) Appl. No.: 19/136,851

(22) PCT Filed: Dec. 7, 2023
(86) PCT No.: PCT/EP2023/084647

§ 371 Date: Jun. 6, 2025

(30) Foreign Application Priority Data
Dec. 8, 2022 (WO) PCT/EP2022/085017

(51) Int. Cl. A61K 39/108; A61K 39/00; A61K 39/385; A61P 37/04

(52) U.S. Cl. CPC A61K 39/0266; A61K 39/385; A61P 37/04; A61K 2039/6037; A61K 2039/70

(57) ABSTRACT

The present invention relates to novel immunogenic compounds comprising at least one antigen of Formula (I), in particular immunogenic compounds of Formula (II), and their use as pharmaceuticals, in particular as vaccines. The invention also concerns related aspects including intermediates, as well as processes for the preparation of the immunogenic compounds. Furthermore, the invention relates to pharmaceutical compositions comprising the immunogenic compounds, as well as the use of the antigen of Formula (I) in biological assays.

This invention was made with United States government support under IDSEP160030-01, IDSEP160030-02 and IDSEP160030-03 awarded by HHS/ASPR awarded by HHS/ASPR. The United States government has certain rights in the invention.

The present invention relates to novel immunogenic compounds comprising at least one antigen of Formula (I), in particular immunogenic compounds of Formula (II), and their use as pharmaceuticals, in particular as vaccines. The invention also concerns related aspects including intermediates, as well as processes for the preparation of the immunogenic compounds. Furthermore, the invention relates to pharmaceutical compositions comprising the immunogenic compounds, as well as the use of the antigen of Formula (I) in biological assays.

Klebsiella pneumoniae (or K. pneumoniae) is a gram-negative, facultative anaerobic, rod-shaped bacterium colonizing mainly respiratory, intestinal and urinary tracts as well as the skin and causing K. pneumoniae infections (KPIs). The bacterium mainly acts as an opportunistic pathogen. KPIs are a major cause of nosocomial infections, primarily affecting immunocompromised patients. Infections caused by K. pneumoniae are an important challenge in healthcare settings due to the emergence of strains resistant to almost all available antimicrobial agents and their worldwide dissemination. Infections caused by K. pneumoniae are responsible for high rates of morbidity and mortality. Thus, prevention of infections caused by K. pneumoniae is highly desirable, and vaccination is the most cost-efficient and the most powerful means to fight KPIs.

K. pneumoniae is an encapsulated bacterium, expressing lipid polysaccharides (LPS) and capsular polysaccharide (CPS, K-antigen) on their outer membrane, which contribute to the virulence of this species.

The LPS consists of three components, namely a lipid A moiety which serves as a membrane anchor, a core oligosaccharide covalently bound to lipid A, and a terminal antigenic polysaccharide comprising repeating saccharide units forming the O-antigen which is covalently bound to the core oligosaccharide. Extracted LPS has been shown to be pyrogenic, toxic and able to cause tissue damage. LPS may be masked by CPS and is usually less exposed to the surface than CPS.

The CPS is comprised of repeating saccharide units that form a layer on the outer bacterial surface. CPS are usually complex, linear or branched, and of larger molecular weight than LPS. Their high immunogenicity and surface exposure had made them interesting targets for vaccination strategies. For instance, WO2016156338 discloses conjugates of synthetic oligosaccharides that are related to carbapenem-resistant K. pneumoniae CPS.

The variability, however, of Klebsiella CPS is high. Serologically more than 77 different CPS types, so-called K-types, K-serotypes or K-antigens have been identified, but there are at least 141 K-types. These additional K-types are identified based on the cps-locus or the K-locus and are called the KL series.

On the other hand, the variability of LPS is lower and the currently known so-called O-types, O-serotypes or O-antigens are limited to 11 major groups: O1, O2a (formerly known as Gal-I), O2ac, O2afg (formerly known as Gal-III), O2aeh (previously O9), O3 (includes sub-serotypes O3, O3a and O3b), O4, O5, O7, O8, and O12. Other than the above 11 additional O-types have been reported based on the O-locus known as the OL series. Though O-antigens are less immunogenic than K-antigens and are exposed to a lesser extent to the surface of the membrane, they have also been considered for vaccination strategies. In a recent survey a large collection of clinical isolates determined the relative prevalence of CPS and LPS serotypes, particularly in multidrug-resistant isolates (e.g. Lam et al., Microbial Genomics 2022; 8:000800; DOI 10.1099/mgen.0.000800).

The O-type O2afg is produced by many multidrug-resistant Klebsiella pneumoniae strains, e.g. ST258. These strains are globally disseminated and extremely drug resistant, for instance carbapenem-resistant. The repeating unit structure of O2afg is for instance disclosed in Kelly et al., J. of Bacteriol., 178(17), 1996, 5205-5214:

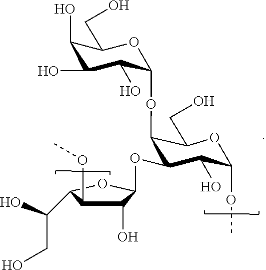

The O-type O2a is one of the five most frequent O-antigens of clinical isolates. Antibiotic resistance is also common in O2a. The repeating unit structure of O2a is for instance disclosed in Kelly et al., J. of Bacteriol., 178(17), 1996, 5205-5214:

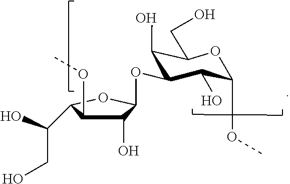

The O2afg serotype is not recognized by O2a-specific antibodies and vice versa (Szijarto et al., Int J Med Microbiol 2016; 306(2):89-98; PMID:26723873;http://dx.doi.org/10.1016/j. ijmm.2015.12.002).

It is known that pure isolated bacterial polysaccharides are thymus-independent antigens that activate B cells in the absence of T cell help. As a consequence, the immune response to carbohydrates is a primary immune response in which antibodies consist mainly of low-affinity IgG, there is no affinity maturation/isotype switching and IgGs are less robust and short-lived. Typical thymus-independent antigens are e.g. CPS from Streptococcus pneumoniae, Haemophilus influenzae type b.

To overcome the limitations of thymus-independent antigens, CPS or O-antigens of LPS can be covalently linked to carrier proteins. Immunization with these polysaccharide-protein conjugates (glycoconjugate vaccines) leads to T cell-dependent B cell activation and can induce long lasting immunity even in infants. For instance, WO2019106201 discloses conjugates of synthetic oligosaccharides related to K. pneumoniae serotype O1, O2, O2ac, and O8 O-polysaccharide and carbapenem-resistant K. pneumoniae ST258 O-polysaccharide.

The underlying mechanism of how conjugate vaccines are presented to T cells is still under debate. Polysaccharide-protein conjugate can be recognized and internalized by polysaccharide-specific B cell receptors (BCRs) of follicular B cells. The protein moiety of the glycoconjugate is processed and presented on MHC-II molecules on the cell surface of the B cell. Recognition of the MHC-II-peptide complex by a peptide-specific T cell then leads to cognate T cell/B cell interactions where the B cell receives activating signals from the T cell. Recent studies suggest that after binding to the BCR and subsequent uptake into endosomes, glycoconjugates may also be processed into glycopeptide fragments. The peptide portion can then bind to MHC-II molecules whereas the carbohydrate (glycan) is exposed to the T cell receptor, where it can interact with carbohydrate-specific CD4+ T cells.

In the case that two different synthetic glycan antigens e.g. glycan “A” and glycan “B” are covalently bound to the same carrier protein molecule, the number of existing glycan “A” and glycan “B”-specific B cells and the affinity of the expressed BCR on these B cells are determining the generation of glycan-specific antibodies (Ab). If, for example, there is a lower number of B cells present for glycan “A”, expressing also low-affinity BCR on the cell surface, the induced Ab response will be dominated by glycan “B”-specific Ab following vaccination. At present, the occurrence and the intensity of immunodominance of glycoconjugates cannot be predicted.

It remains difficult and unpredictable whether or which CPS or LPS may be appropriate candidates or model sequences for vaccines, and in particular it is unpredictable whether and which shorter oligosaccharides would be suitable for generating the desired immune response in vivo. In particular, immunodominance of glycoconjugates remains an unpredictive issue.

To date, there are no approved vaccines available against K. pneumoniae, which demonstrates clearly the challenges associated with the development of such vaccines.

It has now been surprisingly found that new oligosaccharide-antigens have improved properties as potential vaccines against Klebsiella pneumoniae. Due to their specific non-natural hybrid structure targeting multiple serotypes, they are able to reduce the amount of carrier protein per vaccination, thereby reducing undesired carrier induced epitope suppression, and at the same time reducing manufacturing costs.

### DESCRIPTION OF THE FIGURES

In the following the terms “Gal-I” and “O2a” are taken synonymously, and the terms “Gal-III” and “O2afg” are taken synonymously as well.

FIG. 1: Characterization of D13-CRM₁₉₇*, D17-CRM₁₉₇*, and D20-CRM₁₉₇* glycoconjugates in comparison to CRM₁₉₇ by HPLC-SEC.

FIG. 2: SDS-PAGE of D13-CRM₁₉₇*, D17-CRM₁₉₇*, and D20-CRM₁₉₇* glycoconjugates in comparison to CRM₁₉₇ and Marker (protein size marker is GelCode™ Blue Safe Protein Stain (Thermo Scientific)).

FIG. 3: Shows immunogenicity tests in ZiKa rabbits (6 rabbits per group) with 2 μg antigen dose per rabbit per immunization on day 0, 14 and 28 (D13-CRM₁₉₇*) or on day 0, 14 and 34 (D17-CRM₁₉₇*, and D20-CRM₁₉₇*); i.e. FIG. 3A shows the ELISA against Gal-III LPS isolated from the PCM27 strain (Polish Collection of Microorganisms) using an LPS extraction kit (JH Science); FIG. 3B shows ELISA against O2a LPS isolated from the NCTC 9148 strain using an LPS extraction kit (JH Science); sera were diluted 1:100.

FIG. 4: Shows immunogenicity tests in ZiKa rabbits (6 rabbits per group) with 2 μg antigen dose per rabbit per immunization on day 0, 14 and 28 (D13-CRM₁₉₇*) or on day 0, 14 and 34 (D17-CRM₁₉₇*, and D20-CRM₁₉₇*); i.e. FIG. 4A shows the ELISA against inactivated PCM27 bacteria (Gal-III); FIG. 4B shows ELISA against inactivated NCTC 9148 (O2a) bacteria; sera were diluted 1:500 (panel A) or 1:100 (panel B).

FIG. 5: Shows survival data of challenge experiments in mice. CD-1 mice received two intraperitoneal injections of rabbit antisera generated with D13-CRM₁₉₇* (obtained by immunization with 2 μg D13-CRM₁₉₇* antigen dose per rabbit per immunization on days 0, 14 and 28, and collected on day 35) (FIGS. 5A and 5B) or with D17-CRM₁₉₇* or D20-CRM₁₉₇* (FIG. 5C) (obtained by immunization with 2 μg D17-CRM₁₉₇* or D20-CRM₁₉₇* antigen dose per rabbit per immunization on days 0, 14 and 34, and collected on day 41) at −24 h and −1 h relative to infection or control serum of naïve rabbits. At 0 h, the mice were infected intraperitoneally with a lethal dose of a Klebsiella pneumoniae O2a-expressing strain NCTC 9163 (FIG. 5A) or O2afg-expressing strain ST258 (FIGS. 5B and 5C) along with galactosamine treatment (20 mg/mouse intraperitoneally). The mice were observed for 24 h for survival. The indicated survival curves are statistically significantly different with the indicated P values (Log-rank (Mantel-Cox) test). The number of mice per group were: 8 (FIGS. 5A and 5C) or 10 (FIG. 5B).

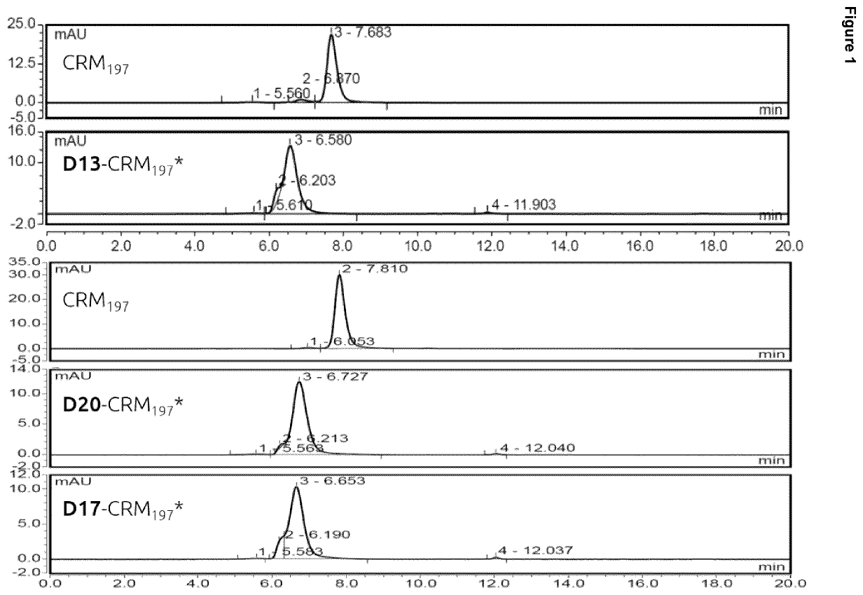

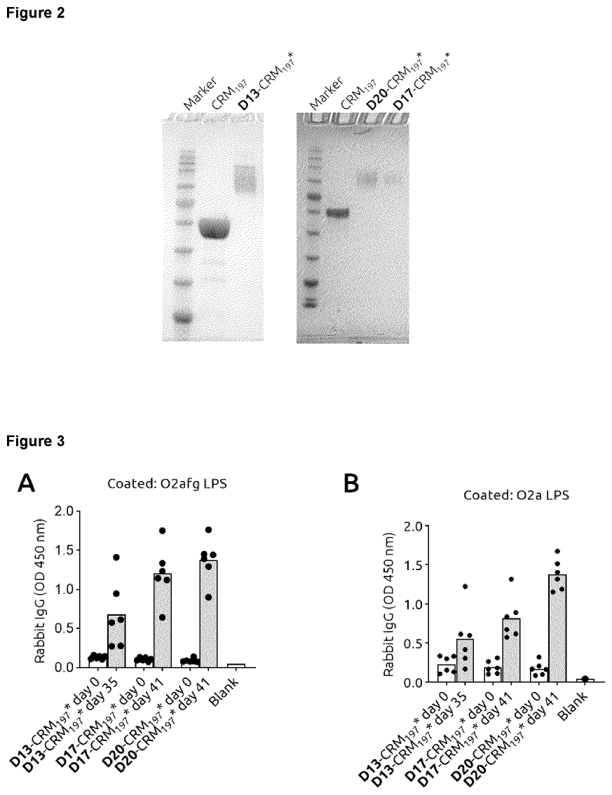

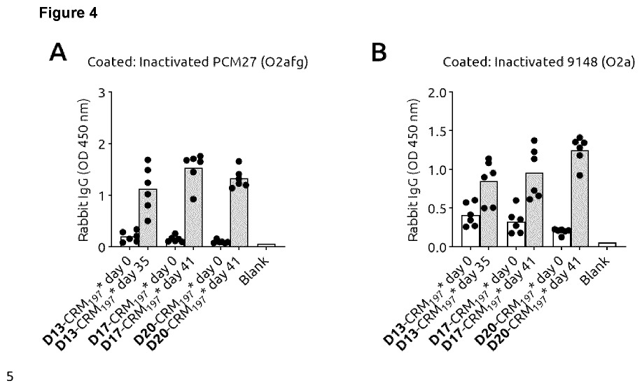

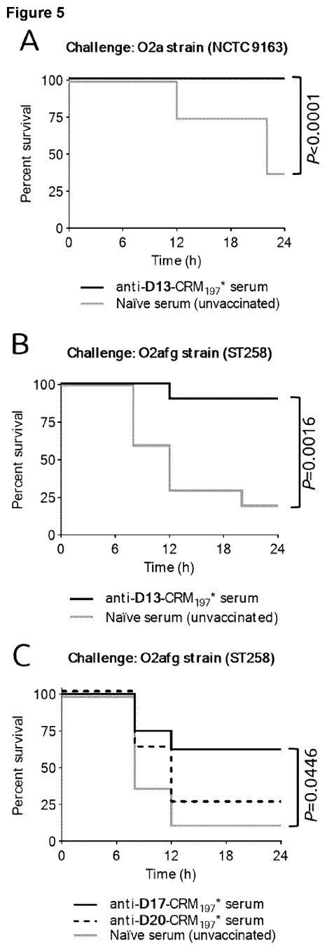

### DETAILED DESCRIPTION OF THE INVENTION

1) In a first aspect, the present invention relates to an immunogenic compound comprising at least one oligosaccharide hybrid antigen having the formula (I)

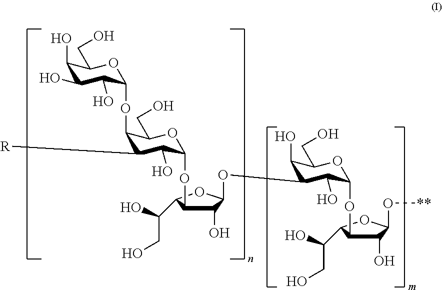

wherein

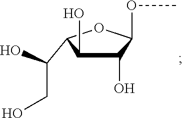

R is OH, or
m is 3, 4, 5, 6, 7 or 8; and
n is 1, 2, 3, 4, 5 or 6;
or a pharmaceutically acceptable salt thereof.

The “**” appointed in the dotted line relates to the point of attachment. It means that at this location, the antigen is attached to a carrier protein via a linker and/or spacer. The oligosaccharide hybrid antigen having formula (I) is responsible for the immunogenic selectivity, i.e. for targeted (specific) antibody response to multiple 0-serotypes of Klebsiella pneumoniae, in particular for O2a and O2afg.

The phrase “at least one antigen” means that the immunogenic compound may comprise one or more antigens according to Formula (I). As an example, the immunogenic compound comprises 1 to 28 antigens according to Formula (I).

In some embodiments, the immunogenic compound may comprise a mixture of different antigens according to Formula (I). Preferred immunogenic compounds are those that have uniform antigens according to Formula (I), i.e. which bear only one specific type of antigen according to Formula (I).

The term “hybrid” means that the antigen is comprised of two different parts of O-serotypes, namely of O2afg (Gal-Ill) and O2a (Gal-I), wherein the O2afg part is distal to the point of attachment “**”, and the O2a is proximal to the point of attachment “**”.

The oligosaccharide of the present invention is composed of galactans, namely
beta-D-galactofuranose/β-D-Galf:

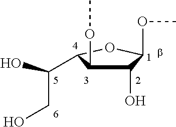

the dotted lines show the point of attachment, namely C1 and C3
alpha-D-galactopyranose/α-D-Galp:

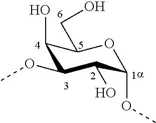

the dotted lines show the point of attachment, namely C1 and C3 Definitions provided herein are intended to apply uniformly to the compounds of Formula (I), (Ia), (II), (IIa), (IIb), (IIc), (III) and (IV) as defined in any one of embodiments 1) to 81), and, mutatis mutandis, throughout the description and the claims unless an otherwise expressly set out definition provides a broader or narrower definition. It is well understood that a definition or preferred definition of a term defines and may replace the respective term independently of (and in combination with) any definition or preferred definition of any or all other terms as defined herein.

The oligosaccharide part (i.e. the antigen, or epitope) of the compounds of Formula (I), (Ia), (II), (IIa), (IIb), (IIc), (III) and (IV) is composed of D-galacto-pyranosides and D-galacto-furanosides, respectively. The configuration at each anomeric center is either alpha or beta. The configuration at the anomeric centers may contribute to a mixture of anomers, whereby the anomers are synthesized in alpha or beta form, preferably as pure alpha or beta anomers. Mixtures of anomers may be separated in a manner known to a person skilled in the art.

2) A further embodiment relates to the immunogenic compound according to embodiment 1), or a pharmaceutically acceptable salt thereof, wherein m is 3, 4, 5 or 6, and n is 2, 3, or 4.

3) A further embodiment relates to the immunogenic compound according to embodiment 1), or a pharmaceutically acceptable salt thereof, wherein m is 3, 4, 5 or 6, and n is 2 or 3, for instance 2.

4) A further embodiment relates to the immunogenic compound according to any one of embodiments 1), 2) or 3), or a pharmaceutically acceptable salt thereof, wherein R is OH.

5) A further embodiment relates to the immunogenic compound according to embodiment 1), or a pharmaceutically acceptable salt thereof, wherein
m is 4, n is 2 and R is OH;
m is 4, n is 2 and R is

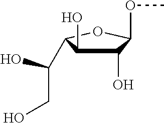

or;
m is 4, n is 3 and R is OH.

6) A further embodiment relates to the immunogenic compound according to embodiment 1), or a pharmaceutically acceptable salt thereof, wherein m is 4, n is 2 and R is OH.

7) A further embodiment relates to the immunogenic compound according to any one of embodiments 1), 2), 3), 4), 5) or 6), or a pharmaceutically acceptable salt thereof, wherein the immunogenic compound further comprises a carrier protein. The carrier protein is preferably non-toxic and suitable to induce immunogenicity. Hence, the carrier protein is preferably a non-toxic carrier protein suitable to induce immunogenicity.

8) A further embodiment relates to the immunogenic compound according to any one of embodiments 1), 2), 3), 4), 5) or 6), or a pharmaceutically acceptable salt thereof, wherein the immunogenic compound further comprises a carrier protein which is selected from the group consisting of
CRM₁₉₇; diphtheria toxoid; tetanus toxoid; cholera toxin B subunit; Neisseria meningitidis outer membrane protein (OMP); capsid protein of bacteriophage Qβ; oligomers or virus-like particles prepared with capsid protein of bacteriophage Qβ; detoxified Exotoxin A of Pseudomonas aeruginosa (EPA); maltose-binding protein (MBP); the Hc fragment of tetanus toxin (TetHc); detoxified hemolysin A of Staphylococcus aureus; clumping factor A (CIfA) and clumping factor B (CIfB) of Staphylococcus aureus; Escherichia coli FimH; Escherichia coli FimHC; detoxified variants of Escherichia coli heat labile enterotoxin; detoxified variants of cholera toxin; Escherichia coli Sat protein; the passenger domain of Escherichia coli Sat protein; detoxified variants of Streptococcus pneumoniae pneumolysin; Campylobacter jejuni AcrA; Pseudomonas PcrV protein; Campylobacter jejuni natural glycoproteins; bovine serum albumin (BSA); pilus protein GBS80 from Group B Streptococcus; Escherichia coli heat-labile enterotoxin; tetanus toxin; cholera toxin; and Streptococcus pneumoniae pneumolysin.

The term “CRM₁₉₇” refers to Cross Reactive Material 197, which is a nontoxic mutant version of the diphtheria toxin, wherein the single amino acid exchange of a glycine (Gly, G) in position 52 to a glutamic acid (Glu, E) renders the protein non-toxic. It is described in more detail in embodiment 17).

The term “Diphtheria toxoid” relates to a formalin-inactivated version of diphtheria toxin having SEQ ID NO: 2 (Uniprot ID: P00587). The present invention encompasses a protein having at least 90%, 91%, 92%, 93%, 94%, 95%, 96%, 97%, 98%, 99% or 99.9% identity to amino acid sequence SEQ ID NO: 2 (preferably 95%, 96%, 97%, 98%, 99% or 99.9% identity to amino acid sequence SEQ ID NO: 2). Diphtheria toxoid can be prepared as described e.g. by Glenny et al. in Br J Exp Pathol. 1923 October; 4(5):283-8 (PMCID: PMC2047731).

The term “Tetanus toxoid” relates to a formalin-inactivated version of tetanus toxin having SEQ ID NO: 3 (Uniprot ID: P04958). The present invention encompasses a protein having at least 90%, 91%, 92%, 93%, 94%, 95%, 96%, 97%, 98%, 99% or 99.9% identity to amino acid sequence SEQ ID NO: 3 (preferably 95%, 96%, 97%, 98%, 99% or 99.9% identity to amino acid sequence SEQ ID NO: 3). Tetanus toxoid can be prepared as described e.g. by G. Ramon at al., CR Soc Biol, 93 (1925), pp. 508-509.

The term “Cholera toxin B subunit” relates to a protein having SEQ ID NO: 4 (Uniprot ID: P01556). The present invention encompasses a protein having at least 90%, 91%, 92%, 93%, 94%, 95%, 96%, 97%, 98%, 99% or 99.9% identity to amino acid sequence SEQ ID NO: 4 (preferably 95%, 96%, 97%, 98%, 99% or 99.9% identity to amino acid sequence SEQ ID NO: 4).

The term “Neisseria meningitidis outer membrane protein” (OMP) relates to a protein having SEQ ID NO: 5 (Uniprot ID: Q51229). The present invention encompasses a protein having at least 90%, 91%, 92%, 93%, 94%, 95%, 96%, 97%, 98%, 99% or 99.9% identity to amino acid sequence SEQ ID NO: 5 (preferably 95%, 96%, 97%, 98%, 99% or 99.9% identity to amino acid sequence SEQ ID NO: 5).

The term “Capsid protein of bacteriophage Qβ” relates to a protein having SEQ ID NO: 6 (Uniprot ID: P03615). The present invention encompasses a protein having at least 90%, 91%, 92%, 93%, 94%, 95%, 96%, 97%, 98%, 99% or 99.9% identity to amino acid sequence SEQ ID NO: 6 (preferably 95%, 96%, 97%, 98%, 99% or 99.9% identity to amino acid sequence SEQ ID NO: 6).

9) A further embodiment relates to the immunogenic compound according to any one of embodiments 1), 2), 3), 4), 5) or 6), or a pharmaceutically acceptable salt thereof, wherein the immunogenic compound further comprises a carrier protein selected from the group consisting of

CRM₁₉₇; diphtheria toxoid; tetanus toxoid; cholera toxin B subunit; Neisseria meningitidis outer membrane protein (OMP); and capsid protein of bacteriophage Qβ. A preferred carrier protein is CRM₁₉₇.

10) A further embodiment relates to the immunogenic compound according to any one of embodiments 7), 8) or 9), wherein the immunogenic compound further comprises a non-immunogenic linker and/or spacer covalently bound to the antigen at the **-binding site at one side, and to the carrier protein at the other side.

11) In a second aspect, the present invention relates to an immunogenic compound of formula (Ia):

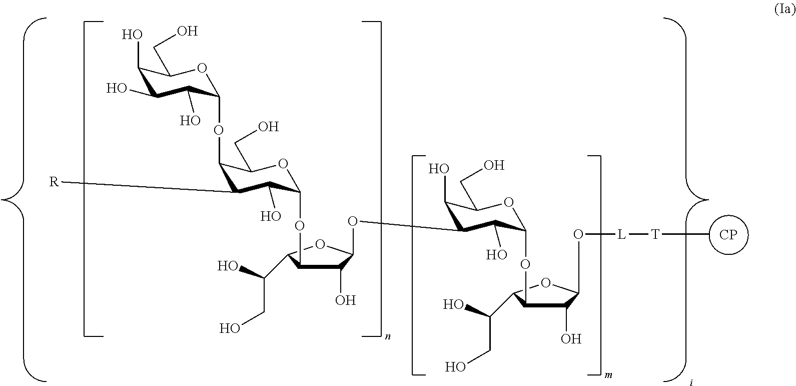

wherein
R is OH, or

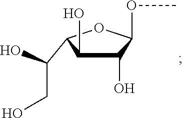

m is 3, 4, 5, 6, 7 or 8; preferably 3, 4, 5 or 6; and
n is 1, 2, 3, 4, 5 or 6; preferably 2, 3, or 4;
i is at least 1, preferably from 1 up to a number corresponding to 90% of the count of lysine residues contained in the carrier protein CP;
-L-T- represents a linker L and a spacer T which together form a bridge having a backbone with a length of 5 to 25 atoms covalently linked together that forms the shortest distance between the oxygen at C1 of the reducing end of the oligosaccharide and the nitrogen of the amino group of a lysine residue at the carrier protein CP, wherein the atoms of the backbone are selected from the group consisting of carbon, nitrogen, oxygen and sulphur; and
CP is a carrier protein selected from the group consisting of
   CRM₁₉₇; diphtheria toxoid; tetanus toxoid; cholera toxin B subunit; Neisseria meningitidis outer membrane protein (OMP); capsid protein of bacteriophage Qβ; oligomers or virus-like particles prepared with capsid protein of bacteriophage Qβ; detoxified Exotoxin A of Pseudomonas aeruginosa (EPA); maltose-binding protein (MBP); the Hc fragment of tetanus toxin (TetHc); detoxified hemolysin A of Staphylococcus aureus; clumping factor A (CIfA) and clumping factor B (CIfB) of Staphylococcus aureus; Escherichia coli FimH; Escherichia coli FimHC; detoxified variants of Escherichia coli heat labile enterotoxin; detoxified variants of cholera toxin; Escherichia coli Sat protein; the passenger domain of Escherichia coli Sat protein; detoxified variants of Streptococcus pneumoniae pneumolysin; Campylobacter jejuni AcrA; Pseudomonas PcrV protein; Campylobacter jejuni natural glycoproteins; bovine serum albumin (BSA); pilus protein GBS80 from Group B Streptococcus; Escherichia coli heat-labile enterotoxin; tetanus toxin; cholera toxin; and Streptococcus pneumoniae pneumolysin;
or a pharmaceutically acceptable salt thereof.

Preferably, i is from 1 up to a number corresponding to 90% of the count of lysine residues contained in the carrier protein CP; more preferably, i is from 1 up to a number corresponding to 75% of the count of lysine residues contained in the carrier protein CP; even more preferably, i is from 1 up to a number corresponding to 40% of the count of lysine residues contained in the carrier protein CP. As an example, in case the carrier protein CP contains 39 lysine residues, the range “from 1 up to a number corresponding to 40% of the count of lysine residues contained in the carrier protein CP” means that i ranges from 1 to 16.

It is explicitly referred to the definition of the carrier protein as disclosed in embodiments 8) and 17).

For the avoidance of any doubt, throughout the present application, the term “lysine residue” and “lysine site” are used synonymously.

The immunogenic compound according to the present invention is an oligosaccharide-carrier protein conjugate, and the term “immunogenic compound” is used synonymously to “oligosaccharide-carrier protein conjugate”.

The immunogenic compound of embodiment 11) may alternatively be referred to as oligosaccharide-carrier protein conjugate of formula (Ia).

The term “hybrid” means that the antigen is comprised of two different parts of O-serotypes, namely of O2afg (Gal-III) and O2a (Gal-I), wherein the O2afg part is distal to the carrier protein, and the O2a is proximal to the carrier protein.

12) A further embodiment relates to the immunogenic compound according to embodiment 11), or a pharmaceutically acceptable salt thereof, wherein R is OH.

13) A further embodiment relates to the immunogenic compound according to embodiment 11), or a pharmaceutically acceptable salt thereof, wherein
m is 4, n is 2 and R is OH;
m is 4, n is 2 and R is

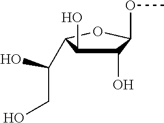

or;
m is 4, n is 3 and R is OH.

14) A further embodiment relates to the immunogenic compound according to embodiment 11), or a pharmaceutically acceptable salt thereof, wherein m is 4, n is 2 and R is OH.

15) A further embodiment relates to the immunogenic compound according to embodiment 11), 12), 13), or 14), or a pharmaceutically acceptable salt thereof, wherein CP is a carrier protein selected from the group consisting of

CRM₁₉₇; diphtheria toxoid; tetanus toxoid; cholera toxin B subunit; Neisseria meningitidis outer membrane protein (OMP); and capsid protein of bacteriophage Qβ (especially CRM₁₉₇).

16) The immunogenic compound according to embodiment 11), 12), 13), 14) or 15) may bear a linker-spacer -L-T- as disclosed in any one of the embodiments 17) to 47). This means that the linker-spacer -L-T- of formula (Ia) is the same as the linker spacer -L-T-described in connection to CRM₁₉₇. Hence, the same description and definitions apply, mutatis mutandis, to the carrier protein CP.

17) In a further aspect, the present invention relates to an immunogenic compound of formula (II)

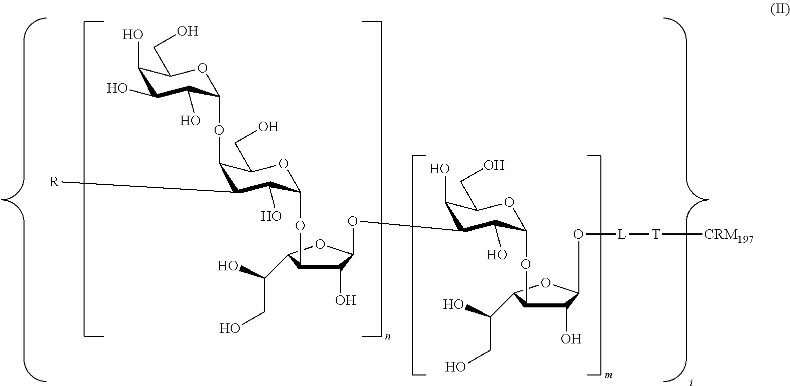

wherein
R is OH, or
m is 3, 4, 5, 6, 7 or 8;
n is 1, 2, 3, 4, 5 or 6;
i is from 1 to 28; and
-L-T- represents a linker L and a spacer T which together form a bridge having a backbone with a length of 5 to 25 atoms covalently linked together that forms the shortest distance between the oxygen at C1 of the reducing end of the oligosaccharide and the nitrogen of the amino group of a lysine residue at the carrier protein CRM₁₉₇, wherein the atoms of the backbone are selected from the group consisting of carbon, nitrogen, oxygen and sulphur;
   or a pharmaceutically acceptable salt thereof.

The term “essentially”, for example when used in a term such as “essentially pure” is understood in the context of the present invention to mean especially that the respective immunogenic compound/oligosaccharide/oligosaccharide-linker compound/oligosaccharide-linker-spacer compound/glycoconjugate consists in an amount of at least 90, especially of at least 95, and notably of at least 99 percent by weight of the respective pure immunogenic compound/oligosaccharide/oligosaccharide-linker compound/oligosaccharide-linker-spacer compound/glycoconjugate.

Whenever a substituent is denoted as optional, it is understood that such substituent may be absent (i.e. the respective residue is unsubstituted with regard to such optional substituent), in which case all positions having a free valency (to which such optional substituent could have been attached to; such as for example in an aromatic ring the ring carbon atoms and/or the ring nitrogen atoms having a free valency) are substituted with hydrogen where appropriate. Likewise, in case the term “optionally” is used in the context of (ring) heteroatom(s), the term means that either the respective optional heteroatom(s), or the like, are absent (i.e. a certain moiety does not contain heteroatom(s)/is a carbocycle/or the like), or the respective optional heteroatom(s), or the like, are present as explicitly defined.

“CRM₁₉₇” refers to Cross Reactive Material 197, which is a nontoxic mutant version of the diphtheria toxin, wherein the single amino acid exchange of a glycine (Gly, G) in position 52 to a glutamic acid (Glu, E) renders the protein non-toxic.

CRM₁₉₇ is produced by C. diphtheriae infected by the nontoxigenic phage β197tox created by nitrosoguanidine mutagenesis of the toxigenic corynephage beta (Uchida et al, J. Biol. Chem., 1973, Vol. 245, No. 11, pp. 3838-3844). The CRM₁₉₇ protein is a safe and effective T-cell dependent carrier for saccharides. CRM₁₉₇ is for instance described by Giannini et al. in Nucleic Acids Research, Vol 12, No. 10, 1984, pp. 4063-4069. Further details about CRM₁₉₇ and production thereof can be found e.g. in U.S. Pat. No. 5,614,382, which are incorporated herein by reference. CRM₁₉₇ may be produced in various expression systems, for instance in Corynebacterium diphtheriae, Escherichia coli or Pseudomonas fluorescens (Hickey et al, J. Pharm. Sci., 2018, 107, 1806-1819).

In the present invention, the term “CRM₁₉₇” encompasses a protein having at least 90%, 91%, 92%, 93%, 94%, 95%, 96%, 97%, 98%, 99%, 99.8% or 99.9% identity to amino acid sequence SEQ ID NO: 1 (preferably at least 90%, 91%, 92%, 93%, 94%, 95%, 96%, 97%, 98%, 99% or 99.9% identity to amino acid sequence SEQ ID NO: 1; and notably at least 95%, 96%, 97%, 98%, 99% or 99.9% identity to amino acid sequence SEQ ID NO: 1), which optionally comprises an additional methionine (Met, M) at the N-terminus, and/or optionally includes residues resulting from functionalizing CRM₁₉₇ at lysine sites, which residues may be in a capped (i.e. deactivated) form.

The phrase “residues resulting from functionalizing CRM₁₉₇ at lysine sites” means that CRM₁₉₇ is functionalized at lysine sites with functional groups suitable for forming a covalent bond to the linker and/or spacer part attached to the antigen, i.e. the oligosaccharide-linker part of the conjugate. Such lysine-functionalized CRM₁₉₇ is known to the skilled person. The functional groups are particularly suitable for linking thiols or for performing click-chemistry. For instance, such functional groups are groups containing a bromo-acetamide, a iodo-acetamide, a maleimide, an azido, or an alkyne group. This means that CRM₁₉₇ optionally includes lysine residues functionalized with a bromo-acetamide, a iodo-acetamide, a maleimide, an azido, or an alkyne group (preferably a bromo-acetamide, a iodo-acetamide, a maleimide group), which groups may be in a capped form.

Preferred functionalized CRM₁₉₇ contains groups carrying bromo-acetamide, iodo-acetamide or maleimide groups, all of them being suitable for reaction with thiol-groups provided by the oligosaccharide/linker moiety. Unreacted functional groups at CRM₁₉₇ may subsequently be quenched with any pharmaceutically acceptable thiol, such as for instance L-cysteine or cysteamine (2-aminoethane-1-thiol) to give the “capped form”.

Preferred lysine-functionalized CRM₁₉₇ is selected from the group consisting of:

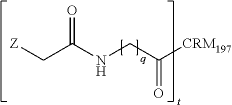

wherein Z is Br or I, q is 2 or 3, and
t is from 1 to 28;

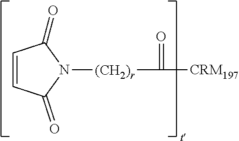

wherein r is 2 or 3, and t′ is from 1 to 28; and

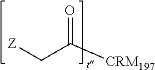

wherein Z is Br or I, and t″ is from 1 to 28.

In a preferred embodiment, CRM₁₉₇ is not functionalized in the above-described way. This means that there is no “pre-functionalization”, but rather, the “natural” lysine residues, i.e. the non-modified amino groups of the lysine residues, are used for directly attaching the oligosaccharide/linker/spacer part thereto.

The amino acid sequence of CRM₁₉₇ is known to the skilled person, and is outlined below as SEQ ID NO:1.

The use of CRM₁₉₇ for the synthesis of saccharide conjugates and preferred conjugation sites on CRM₁₉₇ has been reported (e.g. Möginger et al., Sci. Rep. 6, 20488; doi:10.1038/srep20488 (2016)), which is incorporated herein by reference.

The phrase “-L-T- represents a linker L and a spacer T which together form a bridge having a backbone with a length of 5 to 25 atoms covalently linked together that forms the shortest distance between the oxygen at C1 of the reducing end of the oligosaccharide and the nitrogen of the amino group of a lysine residue at the carrier protein CRM₁₉₇ (or CP, if applicable), wherein the atoms of the backbone are selected from the group consisting of carbon, nitrogen, oxygen and sulphur” means that the backbone may be saturated, unsaturated, unsubstituted or substituted with one or more (especially 1, 2, 3 or 4) substituents independently selected from oxo, (C1-4)alkyl, fluoro, and (C1-2)alkoxy (especially oxo), and optionally a part of a ring structure may be part of the backbone. The ring structure may be a saturated, unsaturated or aromatic 3- to 8-membered ring including condensed ring systems of 2 to 4 rings, wherein the ring atoms are selected from carbon, nitrogen, oxygen and sulphur (especially from carbon and nitrogen), and the ring is unsubstituted or substituted with one or more (especially 1, 2, 3 or 4) substituents independently selected from oxo, (C₁₋₄)alkyl, halogen, and (C₁₋₂)alkoxy (especially oxo).

For avoidance of any doubt, the count of 5 to 25 atoms relates to the count of atoms of the backbone, not of the bridge.

Examples for the optional ring structures that may be part of the backbone are pyrrolidine-2,5-dione, cyclobut-3-ene-1,2-dione, triazole, isoindolin-1-one, 8,9-dihydro-1H-dibenzo[b,f][1,2,3]triazolo[4,5-d]azocine, cyclohexane, and benzene as follows:

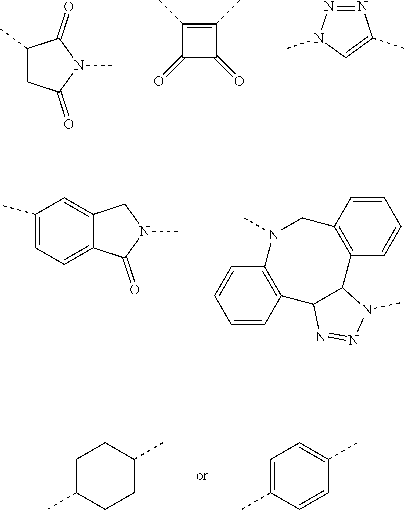

The introduction of these rings or ring systems is known to the skilled person in the field of linker chemistry.

The phrase “the backbone may be unsaturated” means that the backbone chain may contain one or more double bonds, which may or may not be part of a ring system.

For instance, the atom counting in a bridge having a saturated backbone with 3 oxo-substitutions and which backbone is part of a ring system is as follows:

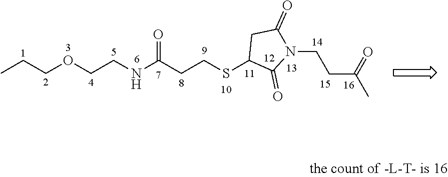 [SMILES: CCCOCCNC(=O)CCSC1CC(=O)N(CCC(C)=O)C1=O]

Hence, the count of the atoms forming the backbone starts with the first atom after the oxygen at C1 and ends with the last atom attached to a lysine nitrogen of CRM₁₉₇.

An oxygen atom in a saturated chain is preferably separated from another oxygen atom by one or more (especially 2, 3, 4 or 5, and notably 2) carbon atoms.

A sulphur atom in a saturated chain is preferably separated from another sulphur atom by one or more (especially 1, 2, 3, 4 or 5) carbon atoms.

The term “halogen” means fluorine, chlorine, or bromine, preferably fluorine or chlorine, more preferably fluorine.

The term “oxo” relates to the functional group ═O, i.e. a substituent oxygen atom connected to another atom (preferably a carbon atom) by a double bond.

The term “alkyl”, used alone or in combination, means a straight or branched saturated hydrocarbon chain containing one to four carbon atoms. The term “(C_{x-y})alkyl” (x and y each being an integer), refers to an alkyl group as defined before containing x to y carbon atoms.

For example a (C₁₋₄)alkyl group contains from one to four carbon atoms. Examples of (C₁₋₄)alkyl groups are methyl, ethyl, n-propyl, iso-propyl, n-butyl, iso-butyl, sec.-butyl and tert.-butyl. Examples of (C₁₋₂)alkyl groups are methyl and ethyl.

The term “alkoxy”, used alone or in combination, refers to an alkyl-O— group wherein the alkyl group is as defined before. The term “(C_{x-y})alkoxy” (x and y each being an integer) refers to an alkoxy group as defined before containing x to y carbon atoms. For example a (C₁₋₂)alkoxy group means a group of the formula (C₁₋₂)alkyl-O— in which the term “(C₁₋₂)alkyl” has the previously given significance. Examples of (C₁₋₂)alkoxy groups are methoxy and ethoxy.

The term “oligosaccharide-carrier protein conjugate” as used herein is taken synonymously to the term “glycoconjugate”. Moreover, the “immunogenic compound” as described herein is an “oligosaccharide-carrier protein conjugate”.

The term “hybrid” means that the antigen is comprised of two different parts of 0-serotypes, namely of O2afg (Gal-Ill) and O2a (Gal-1), wherein the O2afg part is distal to the carrier protein, and the O2a is proximal to the carrier protein.

18) A further embodiment relates to the immunogenic compound according to embodiment 17), or a pharmaceutically acceptable salt thereof, wherein m is 3, 4, 5 or 6, and n is 2, 3, or 4.

19) A further embodiment relates to the immunogenic compound according to embodiment 17), or a pharmaceutically acceptable salt thereof, wherein m is 3, 4, 5 or 6, and n is 2 or 3, for instance 2.

20) A further embodiment relates to the immunogenic compound according to any one of embodiments 17), 18) or 19), or a pharmaceutically acceptable salt thereof, wherein R is OH.

21) A further embodiment relates to the immunogenic compound according to embodiment 17), or a pharmaceutically acceptable salt thereof, wherein
m is 4, n is 2 and R is OH;
m is 4, n is 2 and R is

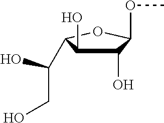

or;
m is 4, n is 3 and R is OH.

22) A further embodiment relates to the immunogenic compound according to embodiment 17), or a pharmaceutically acceptable salt thereof, wherein m is 4, n is 2 and R is OH.

23) A further embodiment relates to the immunogenic compound according to any one of embodiments 11) to 15), and to any one of embodiments 17), 18), 19), 20), 21) or 22), or a pharmaceutically acceptable salt thereof, wherein the bridge does not contain an aromatic or heteroaromatic ring.

24) A further embodiment relates to the immunogenic compound according to any one of embodiments 11) to 15), and to any one of embodiments 17), 18), 19), 20), 21) or 22), or a pharmaceutically acceptable salt thereof, wherein
-L-T- represents a linker L and a spacer T which together form a bridge having a backbone with a length of 5 to 25 atoms covalently linked together that forms the shortest distance between the oxygen at C1 of the reducing end of the oligosaccharide and the nitrogen of the amino group of a lysine residue at the carrier protein CRM₁₉₇ (or CP, if applicable), bearing at most one double bond,
   wherein the atoms of the backbone are selected from the group consisting of carbon, nitrogen, oxygen and sulphur, and
   wherein the backbone may be substituted with one or more (especially 1, 2, 3 or 4) substituents independently selected from oxo, (C₁₋₄)alkyl, fluoro, and (C₁₋₂)alkoxy (especially oxo), and
   wherein a part of the backbone optionally may be part of a 4-, 5- or 6-membered ring selected from:

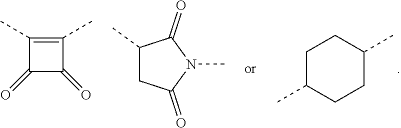

Thereby, the “at most one double bond” is preferably the double bond of the cyclobut-3-ene-1,2-dione ring.

25) A further embodiment relates to the immunogenic compound according to any one of embodiments 11) to 15), and to any one of embodiments 17), 18), 19), 20), 21) or 22), or a pharmaceutically acceptable salt thereof, wherein
-L-T- represents a linker L and a spacer T which together form a bridge which consists of a backbone which is a saturated chain counting from 5 to 25 atoms selected from the group consisting of carbon, nitrogen, oxygen and sulphur (especially carbon, nitrogen and oxygen), which chain may be unsubstituted or substituted with one or more (especially 1, 2, 3 or 4) substituents independently selected from oxo, (C₁₋₄)alkyl, fluoro and (C₁₋₂)alkoxy (especially oxo). This means that the bridge consists of a saturated chain counting from 5 to 25 atoms selected from the group consisting of carbon, nitrogen, oxygen and sulphur (especially carbon, nitrogen and oxygen), which chain may be unsubstituted or substituted with one or more (especially 1, 2, 3 or 4) substituents independently selected from oxo, (C₁₋₄)alkyl, fluoro and (C₁₋₂)alkoxy (especially oxo). For the avoidance of any doubt, in this embodiment, the bridge does not contain a ring structure.

26) A further embodiment relates to the immunogenic compound according to any one of embodiments 11) to 15), and to any one of embodiments 17), 18), 19), 20), 21) or 22), or a pharmaceutically acceptable salt thereof, wherein
-L-T- represents a linker L and a spacer T which together form a bridge which consists of a backbone which is a saturated chain counting from 5 to 25 atoms selected from the group consisting of carbon, nitrogen and oxygen (especially carbon and nitrogen), which chain may be unsubstituted or substituted with one or more (especially 1, 2, 3 or 4) substituents independently selected from oxo, (C₁₋₄)alkyl, fluoro, and (C₁₋₂)alkoxy (especially oxo). This means that the bridge consists of a saturated chain counting from 5 to 25 atoms selected from the group consisting of carbon, nitrogen and oxygen (especially carbon and nitrogen), which chain may be unsubstituted or substituted with one or more (especially 1, 2, 3 or 4) substituents independently selected from oxo, (C₁₋₄)alkyl, fluoro, and (C₁₋₂)alkoxy (especially oxo). For the avoidance of any doubt, in this embodiment, the bridge does not contain a ring structure.

27) A further embodiment relates to the immunogenic compound according to any one of embodiments 11) to 15), and to any one of embodiments 17), 18), 19), 20), 21), 22), 23), 24), 25) or 26) or a pharmaceutically acceptable salt thereof, wherein the backbone of the bridge has a length of 8 to 20, preferably 8 to 16, atoms covalently linked together that forms the shortest distance between the oxygen at C₁ of the reducing end of the oligosaccharide and the nitrogen of the amino group of a lysine residue at the carrier protein CRM₁₉₇ (or CP, if applicable).

28) A further embodiment relates to the immunogenic compound according to any one of embodiments 11) to 15), and to any one of embodiments 17), 18), 19), 20), 21), 22) or 27), or a pharmaceutically acceptable salt thereof, wherein
L represents
   *—(C₂₋₁₀)alkylene-NH—;
   *—(CH₂CH₂O)_{b}—CH₂CH₂NH—, wherein b is 1, 2 or 3;
   *—CH₂CH₂S—CH₂CH₂NH—;
   *—(C₂₋₁₀)fluoroalkylene-NH—;
   *—(CH₂)_{c}NHC(O)(CH₂)_{c′}—NH—, wherein c and c′ are independently from each other from 2 to 6;
   *—(CH₂)_{d}NHC(O)NH(CH₂)_{d′}—NH—, wherein d and d′ are independently from each other from 2 to 6;
   *—(C₁₋₁₀)alkylene-C(O)—NH—(C₂₋₁₀)alkylene-NH—; or
   *—(C₂₋₁₀)alkylene-O—NH—;
T represents
   —C(O)—(C₀₋₁₀)alkylene-C(O)—;
   —C(O)—CH₂CH₂—(OCH₂CH₂)ⱼ—C(O)—, wherein j is from 1 to 5;
   —C(O)—CH₂(CH₂)ₖ—(SCH₂(CH₂)_{k′})_{k″}—C(O)—, wherein k is 0 or 1, k′ is 0 or 1, and k″ is 1, 2, or 3;

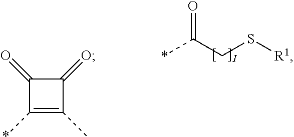

wherein l is 1 or 2; or

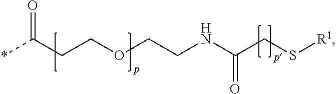

wherein p is from 1 to 4, and p′ is 1 or 2; or
L-T represents
   *—(C₂₋₁₀)alkylene-S—R¹; and
R¹ represents

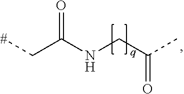

wherein q is 2 or 3;

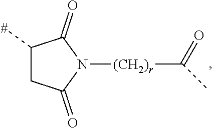

wherein r is 2 or 3; or

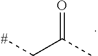

The “*” appointed in the linker L means that at this location, the linker is attached to the oligosaccharide.

The “*” appointed in the spacer T means that at this location, the spacer is attached to the linker L.

The “#” appointed in R¹ means that at this location, R¹ is attached to the sulphur.

The term “—(C_{x-y})alkylene-” (x and y each being an integer), used alone or in combination, refers to a bivalently bound saturated straight or branched hydrocarbon chain containing x to y carbon atoms. For example a (C₂₋₁₀)alkylene group contains from two to ten carbon atoms, and a (C₀₋₁₀)alkylene group is either a bond (i.e. absent, C being zero) or an alkylene group from one to ten carbon atoms. Straight —(C_{x-y})alkylene-, i.e. —(CH₂)_{x-y}— is preferred. Representative examples of (C₂₋₁₀)alkylene groups are ethylene, propylene, butylene, pentylene, hexylene, heptylene, octylene, nonylene and decylene (especially 1,2-ethylene, 1,3-propylene, 1,4-butylene, 1,5-pentylene, 1,6-hexylene, 1,7-heptylene, 1,8-octylene, 1,9-nonylene and 1,10-decylene).

The term “(C_{x-y})fluoroalkylene” (x and y each being an integer), used alone or in combination, refers to a bivalently bound saturated straight or branched chain hydrocarbon group containing x to y carbon atoms in which one or more (and possibly all) hydrogen atoms have been replaced with fluorine. Straight —(C_{x-y})fluoroalkylene- is preferred.

For the avoidance of any doubt, in the present embodiments, the length of the backbone of -L-T- is 5 to 25 atoms, 8 to 20 atoms, or 8 to 16 atoms. This means that the linker L and the spacer T, including R¹ where applicable, together form a bridge having a backbone with a length of 5 to 25 (8 to 20, or 8 to 16) atoms covalently linked together that forms the shortest distance between the oxygen at C₁ of the reducing end of the oligosaccharide and the nitrogen of the amino group of a lysine residue at the carrier protein CRM₁₉₇ (or CP, if applicable). 29) A further embodiment relates to the immunogenic compound according to embodiment 28), or a pharmaceutically acceptable salt thereof, wherein
L represents
   *—(CH₂)ₐ—NH—; wherein a is from 2 to 10;
   *—(CH₂CH₂O)_{b}—CH₂CH₂NH—, wherein b is 1, 2 or 3;
   *—CH₂CH₂S—CH₂CH₂NH—;
   *—(C₂₋₁₀)fluoroalkylene-NH— with fluoroalkylene being a saturated straight chain;
   *—(CH₂)_{c}NHC(O)(CH₂)_{c′}—NH—, wherein c and c′ are independently from each other from 2 to 6;
   *—(CH₂)_{d}NHC(O)NH(CH₂)_{d′}—NH—, wherein d and d′ are independently from each other from 2 to 6;
   *—(CH₂)ₑ—C(O)—NH—(CH₂)_{e′}—NH—; wherein e is from 1 to 10 and e′ is from 2 to 10; or
   *—(CH₂)_{f}—O—NH—, wherein f is from 2 to 10; or
L-T represents
   *—(CH₂)_{g}—S—R, wherein g is from 2 to 10.

30) A further embodiment relates to the immunogenic compound according to embodiment 28), or a pharmaceutically acceptable salt thereof, wherein
L represents
   *—(CH₂)ₐ—NH—; wherein a is from 2 to 10;
   *—(CH₂CH₂O)_{b}—CH₂CH₂NH—, wherein b is 1, 2 or 3;
   *—(C₂₋₁₀)fluoroalkylene-NH— with fluoroalkylene being a saturated straight chain;
   *—(CH₂)ₑ—C(O)—NH—(CH₂)_{e′}—NH—; wherein e is from 1 to 10 and e′ is from 2 to 10; or
   *—(CH₂)_{f}—O—NH—, wherein f is from 2 to 10; or
L-T represents
   *—(CH₂)_{g}—S—R, wherein g is from 2 to 10.

31) A further embodiment relates to the immunogenic compound according to embodiment 28), or a pharmaceutically acceptable salt thereof, wherein
L represents
   *—(CH₂)ₐ—NH—; wherein a is from 2 to 10, preferably from 2 to 6;
   *—(CH₂CH₂O)_{b}—CH₂CH₂NH—, wherein b is 1, 2 or 3, preferably 1 or 2;
   *—(CH₂)ₑ—C(O)—NH—(CH₂)_{e′}—NH—; wherein e is from 1 to 10, preferably from 1 to 6 and e′ is from 2 to 10, preferably from 2 to 6; or
   *—(CH₂)_{f}—O—NH—, wherein f is from 2 to 10, preferably from 2 to 6; or
L-T represents
   *—(CH₂)_{g}—S—R¹, wherein g is from 2 to 10, preferably from 2 to 6.

32) A further embodiment relates to the oligosaccharide-carrier protein conjugate according to embodiment 28), or a pharmaceutically acceptable salt thereof, wherein
L represents
   *—(CH₂)ₐ—NH—; wherein a is from 2 to 10, preferably from 2 to 6;
   *—(CH₂CH₂O)_{b}—CH₂CH₂NH—, wherein b is 1, 2 or 3, preferably 1 or 2; or
   *—(CH₂)_{f}—O—NH—, wherein f is from 2 to 10, preferably from 2 to 6.

33) A further embodiment relates to the immunogenic compound according to embodiment 28), or a pharmaceutically acceptable salt thereof, wherein
L represents
   *—(CH₂)ₐ—NH—; wherein a is from 2 to 10, preferably from 2 to 6; or
   *—(CH₂CH₂O)_{b}—CH₂CH₂NH—, wherein b is 1, 2 or 3, preferably 1 or 2;

34) A further embodiment relates to the immunogenic compound according to embodiment 28), or a pharmaceutically acceptable salt thereof, wherein L represents *—(CH₂)ₐ—NH—; wherein a is from 2 to 10, preferably from 2 to 6.

35) A further embodiment relates to the immunogenic compound according to embodiment 28), or a pharmaceutically acceptable salt thereof, wherein
L represents *—(CH₂)₂—NH—, *—(CH₂)₃—NH—, *—(CH₂)₄—NH—, *—(CH₂)₅—NH—, or *—(CH₂)₆—NH—.

36) A further embodiment relates to the immunogenic compound according to embodiment 28), or a pharmaceutically acceptable salt thereof, wherein L represents *—(CH₂)₅—NH—.

37) A further embodiment relates to the immunogenic compound according to embodiment 28), or a pharmaceutically acceptable salt thereof, wherein
L represents *—(CH₂CH₂O)_{b}—CH₂CH₂NH—, wherein b is 1 or 2; preferably 1.

38) A further embodiment relates to the immunogenic compound according to any one of embodiments 28) to 37), or a pharmaceutically acceptable salt thereof, wherein
T represents
   —C(O)—(CH₂)ₕ—C(O)—, wherein h is from 0 to 10;
   —C(O)—CH₂CH₂—(OCH₂CH₂)ⱼ—C(O)—, wherein j is from 1 to 5;
   —C(O)—CH₂(CH₂)ₖ—(SCH₂(CH₂)_{k′})_{k″}—C(O)—, wherein k is 0 or 1, k′ is 0 or 1, and k″ is 1, 2, or 3

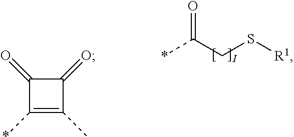

wherein l is 1 or 2; or

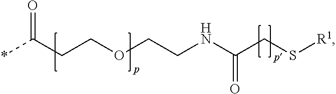

wherein p is from 1 to 4, and p′ is 1 or 2.

39) A further embodiment relates to the immunogenic compound according to any one of embodiments 28) to 37), or a pharmaceutically acceptable salt thereof, wherein
T represents
   —C(O)—(CH₂)ₕ—C(O)—, wherein h is from 0 to 10, preferably from 0 to 6;
   —C(O)—CH₂CH₂—(OCH₂CH₂)ⱼ—C(O)—, wherein j is from 1 to 5, preferably from 1 to 3, more preferably 1;
   —C(O)—CH₂(CH₂)ₖ—(SCH₂(CH₂)_{k′})_{k″}—C(O)—, wherein k is 0 or 1, k′ is 0 or 1, and k″ is 1, 2, or 3, preferably 1; or

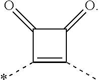

40) A further embodiment relates to the immunogenic compound according to any one of embodiments 28) to 37), or a pharmaceutically acceptable salt thereof, wherein
T represents
   —C(O)—(CH₂)ₕ—C(O)—, wherein h is from 0 to 6;
   —C(O)—CH₂CH₂—(OCH₂CH₂)ⱼ—C(O)—, wherein j is 1 or 2; or
   —C(O)—CH₂(CH₂)ₖ—(SCH₂(CH₂)_{k′})_{k″}—C(O)—, wherein k is 0 or 1, k′ is 0 or 1, and k″ is 1, preferably wherein k and k′ are 0 and k″ is 1.

41) A further embodiment relates to the immunogenic compound according to any one of embodiments 28) to 37), or a pharmaceutically acceptable salt thereof, wherein
T represents
   —C(O)—(CH₂)ₕ—C(O)—, wherein h is 0, 1, 2, 3, 4, 5, or 6, preferably 4; or
   —C(O)—CH₂CH₂—(OCH₂CH₂)ⱼ—C(O)—, wherein j is 1 or 2.

42) A further embodiment relates to the immunogenic compound according to any one of embodiments 28) to 37), or a pharmaceutically acceptable salt thereof, wherein
T represents —C(O)—(CH₂)ₕ—C(O)—, wherein h is 0, 1, 2, 3, 4, 5, or 6, preferably 4.

43) A further embodiment relates to the immunogenic compound according to any one of embodiments 28) to 37), or a pharmaceutically acceptable salt thereof, wherein
T represents

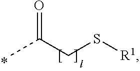

wherein l is 1 or 2; or

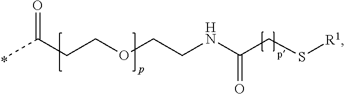

wherein p is from 1 to 4, preferably 1, and p′ is 1 or 2.

44) A further embodiment relates to the immunogenic compound according to any one of embodiments 28) to 37), or a pharmaceutically acceptable salt thereof, wherein
T represents

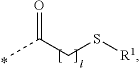

wherein l is 1 or 2.

45) A further embodiment relates to the immunogenic compound according to any one of embodiments 28), 29), 30), 38), 43) or 44), or a pharmaceutically acceptable salt thereof, wherein
R¹ represents

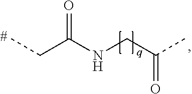

wherein q is 2 or 3;

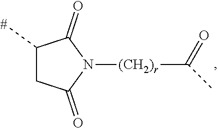

wherein r is 2 or 3; or

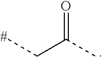

Preferably, R¹ represents:

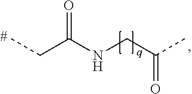

wherein q is 2 or 3; or

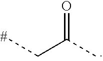

46) A further embodiment relates to the immunogenic compound according to any one of embodiments 11), 12), 13), 14), 15), 17), 18), 19), 20), 21) or 22), or a pharmaceutically acceptable salt thereof, wherein
L represents
   *—(CH₂)₂—NH—, *—(CH₂)₃—NH—, *—(CH₂)₄—NH—, *—(CH₂)₅—NH—, or *—(CH₂)₆—NH—; and
T represents

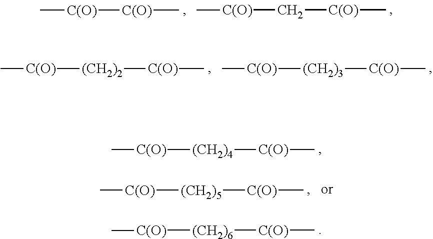

47) A further embodiment relates to the immunogenic compound according to any one of embodiments 11), 12), 13), 14), 15), 17), 18), 19), 20), 21) or 22), or a pharmaceutically acceptable salt thereof, wherein
L represents *—(CH₂)₅—NH— and T represents —C(O)—(CH₂)₄—C(O)—.

48) A preferred embodiment is the immunogenic compound with the following formula (II):

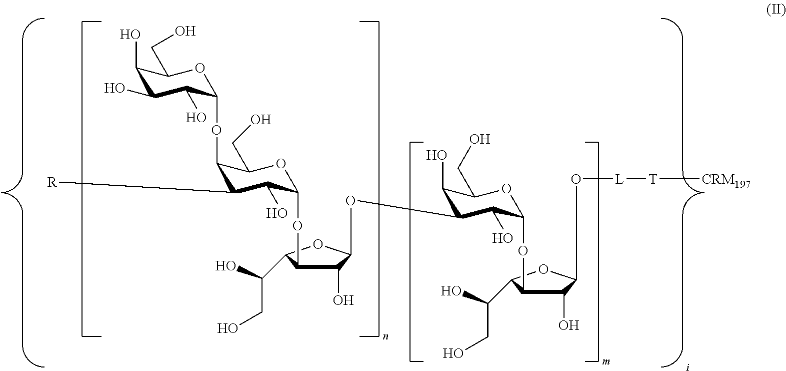

wherein
m is 4, n is 2 and R is OH
m is 4, n is 2 and R is

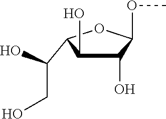

m is 4, n is 3 and R is OH
i is from 1 to 28;
L represents
   *—(CH₂)₂—NH—, *—(CH₂)₃—NH—, *—(CH₂)₄—NH—, *—(CH₂)₅—NH—, or *—(CH₂)₆—NH—; and
T represents

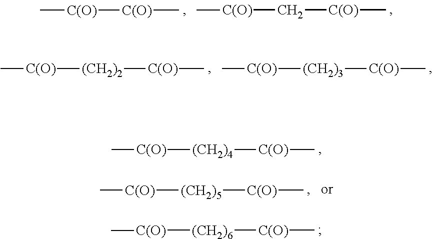

or a pharmaceutically acceptable salt thereof.

49) A further embodiment relates to the immunogenic compound according to embodiment 48), or a pharmaceutically acceptable salt thereof, wherein
m is 4, n is 2 and R is OH;
L represents

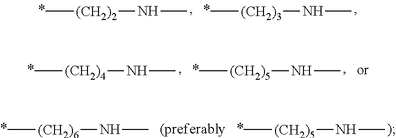

T represents
—C(O)—C(O)—, —C(O)—CH₂—C(O)—, —C(O)—(CH₂)₂—C(O)—, —C(O)—(CH₂)₃—C(O)—, —C(O)—(CH₂)₄—C(O)—, —C(O)—(CH₂)₅—C(O)—, or —C(O)—(CH₂)₆—C(O)— (preferably —C(O)—(CH₂)₄—C(O)—); and
i is from 1 to 28; preferably from 6 to 15.

50) A further embodiment relates to the immunogenic compound according to any one of embodiments 11) to 49), or a pharmaceutically acceptable salt thereof, wherein i is from 1 to 28, 1 to 25, 1 to 23; 1 to 20, 1 to 18, 3 to 25, 3 to 23, 3 to 20, 3 to 18, 5 to 23, 5 to 20, 5 to 18, 6 to 23, 6 to 20, 6 to 18, 6 to 15.

The variable i describes the loading of antigens, i.e. oligosaccharide hybrids on the CRM₁₉₇ protein carrier and is an integer in respect of one single molecule. However, when considering the glycoconjugate as a product of more than one single molecule, it has to be noted that the loading can be described as a statistical distribution, i.e. essentially a Gaussian distribution. The chemical process of producing the product results in a mixture of molecules with such statistical distribution of the loading, and the loading is then provided as the mean of the statistical distribution, in particular the Gaussian distribution.

It is to be understood that for i≥2, m and/or n of the two or more oligosaccharides, that are attached via -L-T- to the carrier protein CP or CRM₁₉₇, respectively, may be the same or different. Preferably, all i oligosaccharides are represented by the same combination of m and n (i.e. have identical structures) or all i oligosaccharides are represented by a first combination of m and n or a second combination of m and n (i.e. have one or another structure); most preferably all i oligosaccharides are represented by the same combination of m and n. The linker-spacer unit -L-T- is identical for the i oligosaccharides of a specific oligosaccharide-carrier protein conjugate.

In other words, preferred compounds are those that have uniform oligosaccharide/linker/spacer residues, i.e. which bear only one specific type of oligosaccharide/linker/spacer residue attached to the carrier protein, preferably to CRM₁₉₇.

51) A further embodiment relates to the immunogenic compound according to any one of embodiments 11) to 49), or a pharmaceutically acceptable salt thereof, wherein i is from 6 to 15.

52) A preferred embodiment relates to the immunogenic compound selected from the group consisting of

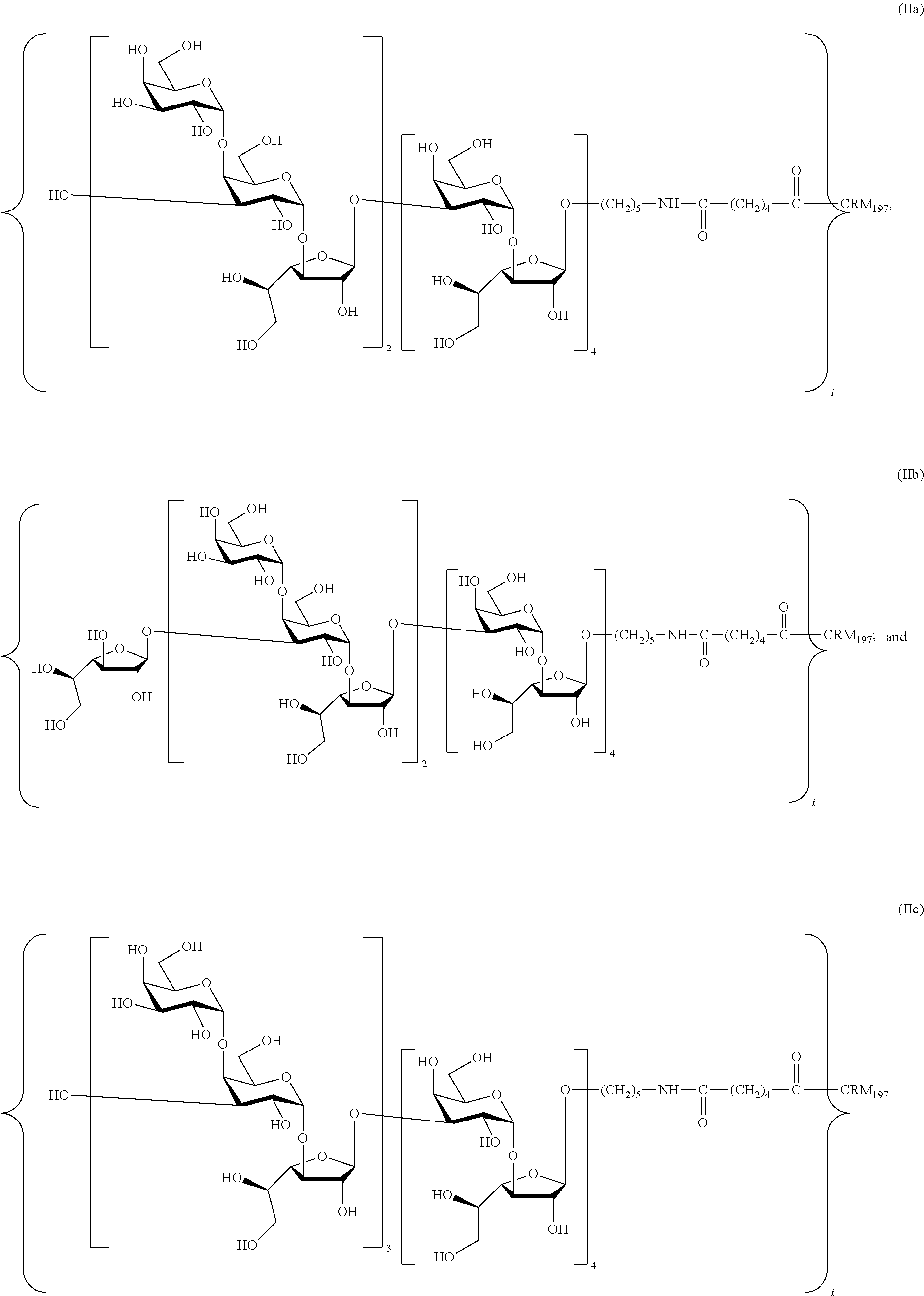

wherein i is from 1 to 28, or a pharmaceutically acceptable salt thereof.

For the avoidance of any doubt, the immunogenic compounds of formulae (IIa), (IIb) and (IIc) according to this embodiment can also be schematically drawn as follows:

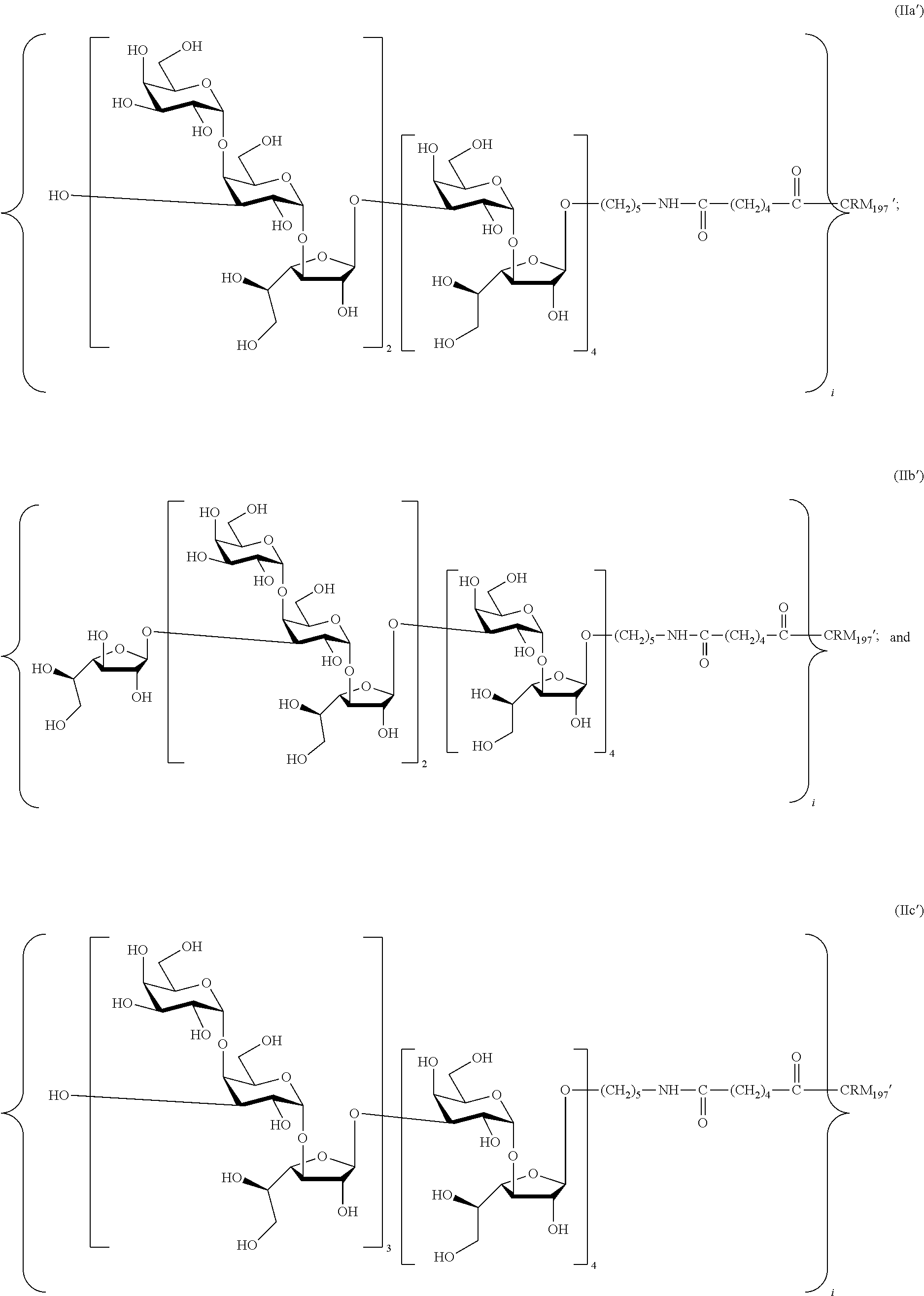

wherein i is from 1 to 28, or a pharmaceutically acceptable salt thereof. CRM₁₉₇′ means CRM₁₉₇ as defined herein, with the only difference in that in formulae (IIa′), (IIb′), and (IIc′), the amino-group of the lysine residue is specifically shown as the attachment position of the linker/spacer part -L-T-.

Preferred values for i are those as disclosed in embodiment 50) or, especially, 51).

53) A further preferred embodiment relates to the immunogenic compound of formula (IIa):

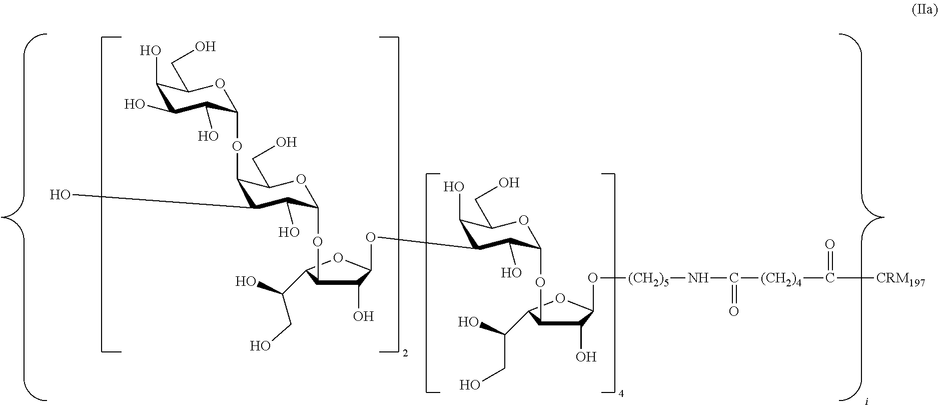

wherein i is from 1 to 28, or a pharmaceutically acceptable salt thereof.

Preferred values for i are those as disclosed in embodiment 50) or, especially, 51).

54) A further preferred embodiment relates to the immunogenic compound selected from the group consisting of:

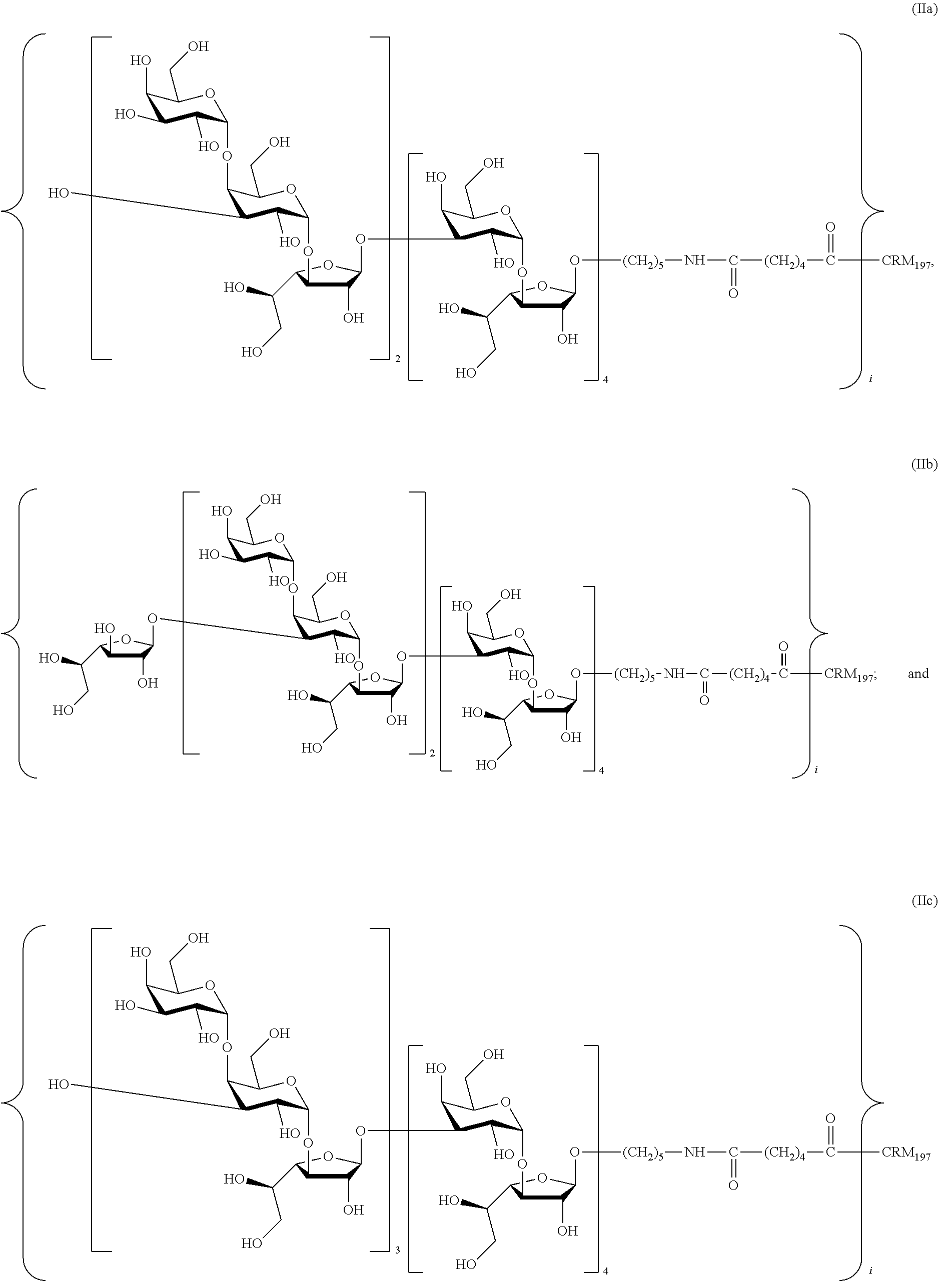

wherein i is from 6 to 15, or a pharmaceutically acceptable salt thereof.

55) A further preferred embodiment relates to the immunogenic compound of formula (IIa):

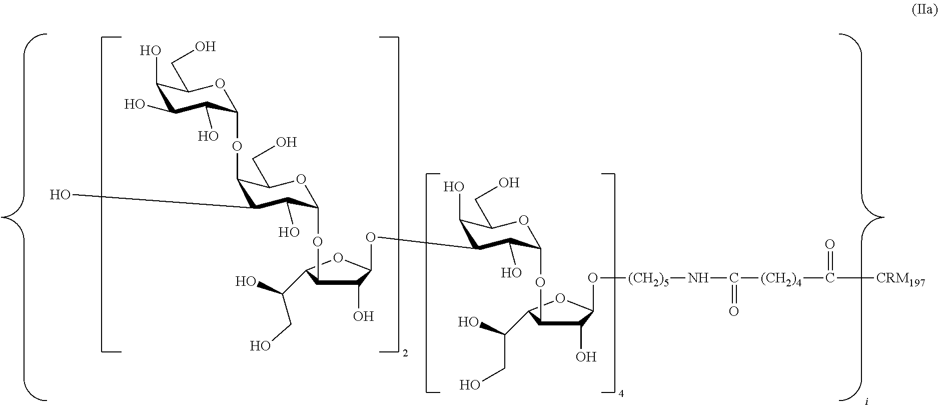

wherein i is from 6 to 15, or a pharmaceutically acceptable salt thereof.

The invention, thus, relates to compounds of the Formula (I) as defined in embodiment 1), and to such compounds further limited by the characteristics of any one of embodiments 2) to 10), to compounds of the Formula (Ia) as defined in embodiment 11), and to such compounds further limited by the characteristics of any one of embodiments 12) to 16), to compounds of Formula (II) as defined in embodiment 17), and to such compounds further limited by the characteristics of any one of embodiments 18) to 55), under consideration of their respective dependencies; to pharmaceutically acceptable salts thereof; and to the use of such compounds as further described below. In particular, compounds of Formula (Ia) and (II) are sub-forms of Formula (I), and compounds of Formula (III) and (IV) are intermediates to prepare compounds of Formula (I), (Ia) and (II), respectively. It is to be understood that the embodiments relating to the definition of L-T as specified in embodiments 23) to 55) in relation to the compound of Formula (II) also apply vice versa as a definition of L-T in respect of a compound of Formula (Ia) according to any one of embodiments 11) to 15).

For avoidance of doubt, especially the following embodiments relating to the compounds of Formula (I) and (II) are thus possible and intended and herewith specifically disclosed in individualized form:

1, 2 + 1, 3 + 1, 4 + 1, 4 + 2 + 1, 4 + 3 + 1, 5 + 1, 6 + 1, 11, 12 + 11, 13 + 11, 14 + 11, 15 + 11, 15 + 12 + 11, 15 + 13 + 11, 15 + 14 + 11,
17, 18 + 17, 19 + 17, 20 + 17, 20 + 18 + 17, 20 + 19 + 17, 21 + 17, 22 + 17, 23 + 11, 23 + 12 + 11, 23 + 13 + 11, 23 + 14 + 11,
23 + 15 + 11, 24 + 11, 24 + 12 + 11, 24 + 13 + 11, 24 + 14 + 11, 24 + 15 + 11, 25 + 11, 25 + 12 + 11, 25 + 13 + 11, 25 + 14 + 11,
25 + 15 + 11, 26 + 11, 26 + 12 + 11, 26 + 13 + 11, 26 + 14 + 11, 26 + 15 + 11, 27 + 11, 27 + 12 + 11, 27 + 13 + 11, 27 + 14 + 11,
27 + 15 + 11, 28 + 11, 28 + 12 + 11, 28 + 13 + 11, 28 + 14 + 11, 28 + 15 + 1129 + 28 + 17, 29 + 28 + 18 + 17, 29 + 28 + 19 + 17,
29 + 28 + 20 + 17, 29 + 28 + 21 + 17, 29 + 28 + 22 + 17, 29 + 28 + 27 + 17, 30 + 28 + 17, 30 + 28 + 18 + 17, 30 + 28 + 19 + 17,
30 + 28 + 20 + 17, 30 + 28 + 21 + 17, 30 + 28 + 22 + 17, 30 + 28 + 27 + 17, 31 + 28 + 17, 31 + 28 + 18 + 17, 31 + 28 + 19 + 17,
31 + 28 + 20 + 17, 31 + 28 + 21 + 17, 31 + 28 + 22 + 17, 31 + 28 + 27 + 17, 32 + 28 + 17, 32 + 28 + 18 + 17, 32 + 28 + 19 + 17,
32 + 28 + 20 + 17, 32 + 28 + 21 + 17, 32 + 28 + 22 + 17, 32 + 28 + 27 + 17, 33 + 28 + 17, 33 + 28 + 18 + 17, 33 + 28 + 19 + 17,
33 + 28 + 20 + 17, 33 + 28 + 21 + 17, 33 + 28 + 22 + 17, 33 + 28 + 27 + 17, 34 + 28 + 17, 34 + 28 + 18 + 17, 34 + 28 + 19 + 17,
34 + 28 + 20 + 17, 34 + 28 + 21 + 17, 34 + 28 + 22 + 17, 34 + 28 + 27 + 17, 35 + 28 + 17, 35 + 28 + 18 + 17, 35 + 28 + 19 + 17,
35 + 28 + 20 + 17, 35 + 28 + 21 + 17, 35 + 28 + 22 + 17, 35 + 28 + 27 + 17, 36 + 28 + 17, 36 + 28 + 18 + 17, 36 + 28 + 19 + 17,
36 + 28 + 20 + 17, 36 + 28 + 21 + 17, 36 + 28 + 22 + 17, 36 + 28 + 27 + 17, 37 + 28 + 17, 37 + 28 + 18 + 17, 37 + 28 + 19 + 17,
37 + 28 + 20 + 17, 37 + 28 + 21 + 17, 37 + 28 + 22 + 17, 37 + 28 + 27 + 17, 38 + 28 + 17, 38 + 28 + 18 + 17, 38 + 28 + 19 + 17,
38 + 28 + 20 + 17, 38 + 28 + 21 + 17, 38 + 28 + 22 + 17, 38 + 28 + 27 + 17, 38 + 29 + 28 + 17,
38 + 29 + 28 + 19 + 17, 38 + 29 + 28 + 20 + 17, 38 + 29 + 28 + 21 + 17, 38 + 29 + 28 + 22 + 17, 38 + 29 + 28 + 27 + 17, 38 + 30 + 28 + 17,
38 + 29 + 28 + 18 + 17, 38 + 30 + 28 + 18 + 17, 38 + 30 + 28 + 19 + 17, 38 + 30 + 28 + 20 + 17, 38 + 30 + 28 + 21 + 17, 38 + 30 + 28 + 22 + 17,
38 + 30 + 28 + 27 + 17, 38 + 31 + 28 + 17, 38 + 31 + 28 + 18 + 17, 38 + 31 + 28 + 19 + 17, 38 + 31 + 28 + 20 + 17, 38 + 31 + 28 + 21 + 17,
38 + 31 + 28 + 22 + 17, 38 + 31 + 28 + 27 + 17, 38 + 32 + 28 + 17, 38 + 32 + 28 + 18 + 17, 38 + 32 + 28 + 19 + 17, 38 + 32 + 28 + 20 + 17,
38 + 32 + 28 + 21 + 17, 38 + 32 + 28 + 22 + 17, 38 + 32 + 28 + 27 + 17, 38 + 33 + 28 + 17, 38 + 33 + 28 + 18 + 17, 38 + 33 + 28 + 19 + 17,
38 + 33 + 28 + 20 + 17, 38 + 33 + 28 + 21 + 17, 38 + 33 + 28 + 22 + 17, 38 + 33 + 28 + 27 + 17, 38 + 34 + 28 + 17, 38 + 34 + 28 + 18 + 17,
38 + 34 + 28 + 19 + 17, 38 + 34 + 28 + 20 + 17, 38 + 34 + 28 + 21 + 17, 38 + 34 + 28 + 22 + 17, 38 + 34 + 28 + 27 + 17, 38 + 35 + 28 + 17,
38 + 35 + 28 + 18 + 17, 38 + 35 + 28 + 19 + 17, 38 + 35 + 28 + 20 + 17, 38 + 35 + 28 + 21 + 17, 38 + 35 + 28 + 22 + 17,
38 + 35 + 28 + 27 + 17, 38 + 36 + 28 + 17, 38 + 36 + 28 + 18 + 17, 38 + 36 + 28 + 19 + 17, 38 + 36 + 28 + 20 + 17, 38 + 36 + 28 + 21 + 17,
38 + 36 + 28 + 22 + 17, 38 + 36 + 28 + 27 + 17, 38 + 37 + 28 + 17, 38 + 37 + 28 + 18 + 17, 38 + 37 + 28 + 19 + 17, 38 + 37 + 28 + 20 + 17,
38 + 37 + 28 + 21 + 17, 38 + 37 + 28 + 22 + 17, 38 + 37 + 28 + 27 + 17, 39 + 28 + 17, 39 + 28 + 18 + 17, 39 + 28 + 19 + 17,
39 + 28 + 20 + 17, 39 + 28 + 21 + 17, 39 + 28 + 22 + 17, 39 + 28 + 27 + 17, 39 + 29 + 28 + 17, 39 + 29 + 28 + 18 + 17,
39 + 29 + 28 + 19 + 17, 39 + 29 + 28 + 20 + 17, 39 + 29 + 28 + 21 + 17, 39 + 29 + 28 + 22 + 17, 39 + 29 + 28 + 27 + 17, 39 + 30 + 28 + 17,
39 + 30 + 28 + 18 + 17, 39 + 30 + 28 + 19 + 17, 39 + 30 + 28 + 20 + 17, 39 + 30 + 28 + 21 + 17, 39 + 30 + 28 + 22 + 17,
39 + 30 + 28 + 27 + 17, 39 + 31 + 28 + 17, 39 + 31 + 28 + 18 + 17, 39 + 31 + 28 + 19 + 17, 39 + 31 + 28 + 20 + 17, 39 + 31 + 28 + 21 + 17,
39 + 31 + 28 + 22 + 17, 39 + 31 + 28 + 27 + 17, 39 + 32 + 28 + 17, 39 + 32 + 28 + 18 + 17, 39 + 32 + 28 + 19 + 17, 39 + 32 + 28 + 20 + 17,
39 + 32 + 28 + 21 + 17, 39 + 32 + 28 + 22 + 17, 39 + 32 + 28 + 27 + 17, 39 + 33 + 28 + 17, 39 + 33 + 28 + 18 + 17, 39 + 33 + 28 + 19 + 17,
39 + 33 + 28 + 20 + 17, 39 + 33 + 28 + 21 + 17, 39 + 33 + 28 + 22 + 17, 39 + 33 + 28 + 27 + 17, 39 + 34 + 28 + 17, 39 + 34 + 28 + 18 + 17,
39 + 34 + 28 + 19 + 17, 39 + 34 + 28 + 20 + 17, 39 + 34 + 28 + 21 + 17, 39 + 34 + 28 + 22 + 17, 39 + 34 + 28 + 27 + 17, 39 + 35 + 28 + 17,
39 + 35 + 28 + 18 + 17, 39 + 35 + 28 + 19 + 17, 39 + 35 + 28 + 20 + 17, 39 + 35 + 28 + 21 + 17, 39 + 35 + 28 + 22 + 17,
39 + 35 + 28 + 27 + 17, 39 + 36 + 28 + 17, 39 + 36 + 28 + 18 + 17, 39 + 36 + 28 + 19 + 17, 39 + 36 + 28 + 20 + 17, 39 + 36 + 28 + 21 + 17,
39 + 36 + 28 + 22 + 17, 39 + 36 + 28 + 27 + 17, 39 + 37 + 28 + 17, 39 + 37 + 28 + 18 + 17, 39 + 37 + 28 + 19 + 17, 39 + 37 + 28 + 20 + 17,
39 + 37 + 28 + 21 + 17, 39 + 37 + 28 + 22 + 17, 39 + 37 + 28 + 27 + 17, 40 + 28 + 17, 40 + 28 + 18 + 17,
40 + 28 + 19 + 17, 40 + 28 + 20 + 17, 40 + 28 + 21 + 17, 40 + 28 + 22 + 17, 40 + 28 + 27 + 17, 40 + 29 + 28 + 17, 40 + 29 + 28 + 18 + 17,
40 + 29 + 28 + 19 + 17, 40 + 29 + 28 + 20 + 17, 40 + 29 + 28 + 21 + 17, 40 + 29 + 28 + 22 + 17, 40 + 29 + 28 + 27 + 17, 40 + 30 + 28 + 17,
40 + 30 + 28 + 18 + 17, 40 + 30 + 28 + 19 + 17, 40 + 30 + 28 + 20 + 17, 40 + 30 + 28 + 21 + 17, 40 + 30 + 28 + 22 + 17,
40 + 30 + 28 + 27 + 17, 40 + 31 + 28 + 17, 40 + 31 + 28 + 18 + 17, 40 + 31 + 28 + 19 + 17, 40 + 31 + 28 + 20 + 17, 40 + 31 + 28 + 21 + 17,
40 + 31 + 28 + 22 + 17, 40 + 31 + 28 + 27 + 17, 40 + 32 + 28 + 17, 40 + 32 + 28 + 18 + 17, 40 + 32 + 28 + 19 + 17, 40 + 32 + 28 + 20 + 17,
40 + 32 + 28 + 21 + 17, 40 + 32 + 28 + 22 + 17, 40 + 32 + 28 + 27 + 17, 40 + 33 + 28 + 17, 40 + 33 + 28 + 18 + 17, 40 + 33 + 28 + 19 + 17,
40 + 33 + 28 + 20 + 17, 40 + 33 + 28 + 21 + 17, 40 + 33 + 28 + 22 + 17, 40 + 33 + 28 + 27 + 17, 40 + 34 + 28 + 17, 40 + 34 + 28 + 18 + 17,
40 + 34 + 28 + 19 + 17, 40 + 34 + 28 + 20 + 17, 40 + 34 + 28 + 21 + 17, 40 + 34 + 28 + 22 + 17, 40 + 34 + 28 + 27 + 17, 40 + 35 + 28 + 17,
40 + 35 + 28 + 18 + 17, 40 + 35 + 28 + 19 + 17, 40 + 35 + 28 + 20 + 17, 40 + 35 + 28 + 21 + 17, 40 + 35 + 28 + 22 + 17,
40 + 35 + 28 + 27 + 17, 40 + 36 + 28 + 17, 40 + 36 + 28 + 18 + 17, 40 + 36 + 28 + 19 + 17, 40 + 36 + 28 + 20 + 17, 40 + 36 + 28 + 21 + 17,
40 + 36 + 28 + 22 + 17, 40 + 36 + 28 + 27 + 17, 40 + 37 + 28 + 17, 40 + 37 + 28 + 18 + 17, 40 + 37 + 28 + 19 + 17, 40 + 37 + 28 + 20 + 17,
40 + 37 + 28 + 21 + 17, 40 + 37 + 28 + 22 + 17, 40 + 37 + 28 + 27 + 17, 41 + 28 + 17, 41 + 28 + 18 + 17, 41 + 28 + 19 + 17,
41 + 28 + 20 + 17, 41 + 28 + 21 + 17, 41 + 28 + 22 + 17, 41 + 28 + 27 + 17, 41 + 29 + 28 + 17, 41 + 29 + 28 + 18 + 17,
41 + 29 + 28 + 19 + 17, 41 + 29 + 28 + 20 + 17, 41 + 29 + 28 + 21 + 17, 41 + 29 + 28 + 22 + 17, 41 + 29 + 28 + 27 + 17, 41 + 30 + 28 + 17,
41 + 30 + 28 + 18 + 17, 41 + 30 + 28 + 19 + 17, 41 + 30 + 28 + 20 + 17, 41 + 30 + 28 + 21 + 17, 41 + 30 + 28 + 22 + 17,
41 + 30 + 28 + 27 + 17, 41 + 31 + 28 + 17, 41 + 31 + 28 + 18 + 17, 41 + 31 + 28 + 19 + 17, 41 + 31 + 28 + 20 + 17, 41 + 31 + 28 + 21 + 17,
41 + 31 + 28 + 22 + 17, 41 + 31 + 28 + 27 + 17, 41 + 32 + 28 + 17, 41 + 32 + 28 + 18 + 17, 41 + 32 + 28 + 19 + 17, 41 + 32 + 28 + 20 + 17,
41 + 32 + 28 + 21 + 17, 41 + 32 + 28 + 22 + 17, 41 + 32 + 28 + 27 + 17, 41 + 33 + 28 + 17, 41 + 33 + 28 + 18 + 17, 41 + 33 + 28 + 19 + 17,
41 + 33 + 28 + 20 + 17, 41 + 33 + 28 + 21 + 17, 41 + 33 + 28 + 22 + 17, 41 + 33 + 28 + 27 + 17, 41 + 34 + 28 + 17, 41 + 34 + 28 + 18 + 17,
41 + 34 + 28 + 19 + 17, 41 + 34 + 28 + 20 + 17, 41 + 34 + 28 + 21 + 17, 41 + 34 + 28 + 22 + 17, 41 + 34 + 28 + 27 + 17, 41 + 35 + 28 + 17,
41 + 35 + 28 + 18 + 17, 41 + 35 + 28 + 19 + 17, 41 + 35 + 28 + 20 + 17, 41 + 35 + 28 + 21 + 17, 41 + 35 + 28 + 22 + 17,
41 + 35 + 28 + 27 + 17, 41 + 36 + 28 + 17, 41 + 36 + 28 + 18 + 17, 41 + 36 + 28 + 19 + 17, 41 + 36 + 28 + 20 + 17, 41 + 36 + 28 + 21 + 17,
41 + 36 + 28 + 22 + 17, 41 + 36 + 28 + 27 + 17, 41 + 37 + 28 + 17, 41 + 37 + 28 + 18 + 17, 41 + 37 + 28 + 19 + 17, 41 + 37 + 28 + 20 + 17,
41 + 37 + 28 + 21 + 17, 41 + 37 + 28 + 22 + 17, 41 + 37 + 28 + 27 + 17, 42 + 28 + 17, 42 + 28 + 18 + 17, 42 + 28 + 19 + 17,
42 + 28 + 20 + 17, 42 + 28 + 21 + 17, 42 + 28 + 22 + 17, 42 + 28 + 27 + 17, 42 + 29 + 28 + 17, 42 + 29 + 28 + 18 + 17,
42 + 29 + 28 + 19 + 17, 42 + 29 + 28 + 20 + 17, 42 + 29 + 28 + 21 + 17, 42 + 29 + 28 + 22 + 17, 42 + 29 + 28 + 27 + 17, 42 + 30 + 28 + 17,
42 + 30 + 28 + 18 + 17, 42 + 30 + 28 + 19 + 17, 42 + 30 + 28 + 20 + 17, 42 + 30 + 28 + 21 + 17, 42 + 30 + 28 + 22 + 17,
42 + 30 + 28 + 27 + 17, 42 + 31 + 28 + 17, 42 + 31 + 28 + 18 + 17, 42 + 31 + 28 + 19 + 17, 42 + 31 + 28 + 20 + 17, 42 + 31 + 28 + 21 + 17,
42 + 31 + 28 + 22 + 17, 42 + 31 + 28 + 27 + 17, 42 + 32 + 28 + 17, 42 + 32 + 28 + 18 + 17, 42 + 32 + 28 + 19 + 17, 42 + 32 + 28 + 20 + 17,
42 + 32 + 28 + 21 + 17, 42 + 32 + 28 + 22 + 17, 42 + 32 + 28 + 27 + 17, 42 + 33 + 28 + 17, 42 + 33 + 28 + 18 + 17, 42 + 33 + 28 + 19 + 17,
42 + 33 + 28 + 20 + 17, 42 + 33 + 28 + 21 + 17, 42 + 33 + 28 + 22 + 17, 42 + 33 + 28 + 27 + 17, 42 + 34 + 28 + 17, 42 + 34 + 28 + 18 + 17,
42 + 34 + 28 + 19 + 17, 42 + 34 + 28 + 20 + 17, 42 + 34 + 28 + 21 + 17, 42 + 34 + 28 + 22 + 17, 42 + 34 + 28 + 27 + 17, 42 + 35 + 28 + 17,
42 + 35 + 28 + 18 + 17, 42 + 35 + 28 + 19 + 17, 42 + 35 + 28 + 20 + 17, 42 + 35 + 28 + 21 + 17, 42 + 35 + 28 + 22 + 17,
42 + 35 + 28 + 27 + 17, 42 + 36 + 28 + 17, 42 + 36 + 28 + 18 + 17, 42 + 36 + 28 + 19 + 17, 42 + 36 + 28 + 20 + 17, 42 + 36 + 28 + 21 + 17,
42 + 36 + 28 + 22 + 17, 42 + 36 + 28 + 27 + 17, 42 + 37 + 28 + 17, 42 + 37 + 28 + 18 + 17, 42 + 37 + 28 + 19 + 17, 42 + 37 + 28 + 20 + 17,
42 + 37 + 28 + 21 + 17, 42 + 37 + 28 + 22 + 17, 42 + 37 + 28 + 27 + 17, 43 + 28 + 17, 43 + 28 + 18 + 17, 43 + 28 + 19 + 17,
43 + 28 + 20 + 17, 43 + 28 + 21 + 17, 43 + 28 + 22 + 17, 43 + 28 + 27 + 17, 43 + 29 + 28 + 17, 43 + 29 + 28 + 18 + 17,
43 + 29 + 28 + 19 + 17, 43 + 29 + 28 + 20 + 17, 43 + 29 + 28 + 21 + 17, 43 + 29 + 28 + 22 + 17, 43 + 29 + 28 + 27 + 17, 43 + 30 + 28 + 17,
43 + 30 + 28 + 18 + 17, 43 + 30 + 28 + 19 + 17, 43 + 30 + 28 + 20 + 17, 43 + 30 + 28 + 21 + 17, 43 + 30 + 28 + 22 + 17,
43 + 30 + 28 + 27 + 17, 43 + 31 + 28 + 17, 43 + 31 + 28 + 18 + 17, 43 + 31 + 28 + 19 + 17, 43 + 31 + 28 + 20 + 17, 43 + 31 + 28 + 21 + 17,
43 + 31 + 28 + 22 + 17, 43 + 31 + 28 + 27 + 17, 43 + 32 + 28 + 17, 43 + 32 + 28 + 18 + 17, 43 + 32 + 28 + 19 + 17, 43 + 32 + 28 + 20 + 17,
43 + 32 + 28 + 21 + 17, 43 + 32 + 28 + 22 + 17, 43 + 32 + 28 + 27 + 17, 43 + 33 + 28 + 17, 43 + 33 + 28 + 18 + 17, 43 + 33 + 28 + 19 + 17,
43 + 33 + 28 + 20 + 17, 43 + 33 + 28 + 21 + 17, 43 + 33 + 28 + 22 + 17, 43 + 33 + 28 + 27 + 17, 43 + 34 + 28 + 17, 43 + 34 + 28 + 18 + 17,
43 + 34 + 28 + 19 + 17, 43 + 34 + 28 + 20 + 17, 43 + 34 + 28 + 21 + 17, 43 + 34 + 28 + 22 + 17, 43 + 34 + 28 + 27 + 17, 43 + 35 + 28 + 17,
43 + 35 + 28 + 18 + 17, 43 + 35 + 28 + 19 + 17, 43 + 35 + 28 + 20 + 17, 43 + 35 + 28 + 21 + 17, 43 + 35 + 28 + 22 + 17,
43 + 35 + 28 + 27 + 17, 43 + 36 + 28 + 17, 43 + 36 + 28 + 18 + 17, 43 + 36 + 28 + 19 + 17, 43 + 36 + 28 + 20 + 17, 43 + 36 + 28 + 21 + 17,
43 + 36 + 28 + 22 + 17, 43 + 36 + 28 + 27 + 17, 43 + 37 + 28 + 17, 43 + 37 + 28 + 18 + 17, 43 + 37 + 28 + 19 + 17, 43 + 37 + 28 + 20 + 17,
43 + 37 + 28 + 21 + 17, 43 + 37 + 28 + 22 + 17, 43 + 37 + 28 + 27 + 17, 44 + 28 + 17, 44 + 28 + 18 + 17, 44 + 28 + 19 + 17,
44 + 28 + 20 + 17, 44 + 28 + 21 + 17, 44 + 28 + 22 + 17, 44 + 28 + 27 + 17, 44 + 29 + 28 + 17, 44 + 29 + 28 + 18 + 17,
44 + 29 + 28 + 19 + 17, 44 + 29 + 28 + 20 + 17, 44 + 29 + 28 + 21 + 17, 44 + 29 + 28 + 22 + 17, 44 + 29 + 28 + 27 + 17, 44 + 30 + 28 + 17,
44 + 30 + 28 + 18 + 17, 44 + 30 + 28 + 19 + 17, 44 + 30 + 28 + 20 + 17, 44 + 30 + 28 + 21 + 17, 44 + 30 + 28 + 22 + 17,
44 + 30 + 28 + 27 + 17, 44 + 31 + 28 + 17, 44 + 31 + 28 + 18 + 17, 44 + 31 + 28 + 19 + 17, 44 + 31 + 28 + 20 + 17, 44 + 31 + 28 + 21 + 17,
44 + 31 + 28 + 22 + 17, 44 + 31 + 28 + 27 + 17, 44 + 32 + 28 + 17, 44 + 32 + 28 + 18 + 17, 44 + 32 + 28 + 19 + 17, 44 + 32 + 28 + 20 + 17,
44 + 32 + 28 + 21 + 17, 44 + 32 + 28 + 22 + 17, 44 + 32 + 28 + 27 + 17, 44 + 33 + 28 + 17, 44 + 33 + 28 + 18 + 17, 44 + 33 + 28 + 19 + 17,
44 + 33 + 28 + 20 + 17, 44 + 33 + 28 + 21 + 17, 44 + 33 + 28 + 22 + 17, 44 + 33 + 28 + 27 + 17, 44 + 34 + 28 + 17, 44 + 34 + 28 + 18 + 17,
44 + 34 + 28 + 19 + 17, 44 + 34 + 28 + 20 + 17, 44 + 34 + 28 + 21 + 17, 44 + 34 + 28 + 22 + 17, 44 + 34 + 28 + 27 + 17, 44 + 35 + 28 + 17,
44 + 35 + 28 + 18 + 17, 44 + 35 + 28 + 19 + 17, 44 + 35 + 28 + 20 + 17, 44 + 35 + 28 + 21 + 17, 44 + 35 + 28 + 22 + 17,
44 + 35 + 28 + 27 + 17, 44 + 36 + 28 + 17, 44 + 36 + 28 + 18 + 17, 44 + 36 + 28 + 19 + 17, 44 + 36 + 28 + 20 + 17, 44 + 36 + 28 + 21 + 17,
44 + 36 + 28 + 22 + 17, 44 + 36 + 28 + 27 + 17, 44 + 37 + 28 + 17, 44 + 37 + 28 + 18 + 17, 44 + 37 + 28 + 19 + 17, 44 + 37 + 28 + 20 + 17,
44 + 37 + 28 + 21 + 17, 44 + 37 + 28 + 22 + 17, 44 + 37 + 28 + 27 + 17, 45 + 28 + 17, 45 + 28 + 18 + 17,
45 + 28 + 19 + 17, 45 + 28 + 20 + 17, 45 + 28 + 21 + 17, 45 + 28 + 22 + 17, 45 + 28 + 27 + 17, 45 + 29 + 28 + 17, 45 + 29 + 28 + 18 + 17,
45 + 29 + 28 + 19 + 17, 45 + 29 + 28 + 20 + 17, 45 + 29 + 28 + 21 + 17, 45 + 29 + 28 + 22 + 17, 45 + 29 + 28 + 27 + 17, 45 + 30 + 28 + 17,
45 + 30 + 28 + 18 + 17, 45 + 30 + 28 + 19 + 17, 45 + 30 + 28 + 20 + 17, 45 + 30 + 28 + 21 + 17, 45 + 30 + 28 + 22 + 17,
45 + 30 + 28 + 27 + 17, 45 + 38 + 28 + 17, 45 + 38 + 28 + 18 + 17, 45 + 38 + 28 + 19 + 17, 45 + 38 + 28 + 20 + 17, 45 + 38 + 28 + 21 + 17,
45 + 38 + 28 + 22 + 17, 45 + 38 + 28 + 27 + 17, 45 + 38 + 29 + 28 + 17, 45 + 38 + 29 + 28 + 18 + 17, 45 + 38 + 29 + 28 + 19 + 17,
45 + 38 + 29 + 28 + 20 + 17, 45 + 38 + 29 + 28 + 21 + 17, 45 + 38 + 29 + 28 + 22 + 17, 45 + 38 + 29 + 28 + 27 + 17, 45 + 38 + 30 + 28 + 17,
45 + 38 + 30 + 28 + 18 + 17, 45 + 38 + 30 + 28 + 19 + 17, 45 + 38 + 30 + 28 + 20 + 17, 45 + 38 + 30 + 28 + 21 + 17,
45 + 38 + 30 + 28 + 22 + 17, 45 + 38 + 30 + 28 + 27 + 17, 45 + 38 + 31 + 28 + 17, 45 + 38 + 31 + 28 + 18 + 17, 45 + 38 + 31 + 28 + 19 + 17,
45 + 38 + 31 + 28 + 20 + 17, 45 + 38 + 31 + 28 + 21 + 17, 45 + 38 + 31 + 28 + 22 + 17, 45 + 38 + 31 + 28 + 27 + 17, 45 + 38 + 32 + 28 + 17,
45 + 38 + 32 + 28 + 18 + 17, 45 + 38 + 32 + 28 + 19 + 17, 45 + 38 + 32 + 28 + 20 + 17, 45 + 38 + 32 + 28 + 21 + 17,
45 + 38 + 32 + 28 + 22 + 17, 45 + 38 + 32 + 28 + 27 + 17, 45 + 38 + 33 + 28 + 17, 45 + 38 + 33 + 28 + 18 + 17, 45 + 38 + 33 + 28 + 19 + 17,
45 + 38 + 33 + 28 + 20 + 17, 45 + 38 + 33 + 28 + 21 + 17, 45 + 38 + 33 + 28 + 22 + 17, 45 + 38 + 33 + 28 + 27 + 17, 45 + 38 + 34 + 28 + 17,
45 + 38 + 34 + 28 + 18 + 17, 45 + 38 + 34 + 28 + 19 + 17, 45 + 38 + 34 + 28 + 20 + 17, 45 + 38 + 34 + 28 + 21 + 17,
45 + 38 + 34 + 28 + 22 + 17, 45 + 38 + 34 + 28 + 27 + 17, 45 + 38 + 35 + 28 + 17, 45 + 38 + 35 + 28 + 18 + 17, 45 + 38 + 35 + 28 + 19 + 17,
45 + 38 + 35 + 28 + 20 + 17, 45 + 38 + 35 + 28 + 21 + 17, 45 + 38 + 35 + 28 + 22 + 17, 45 + 38 + 35 + 28 + 27 + 17, 45 + 38 + 36 + 28 + 17,
45 + 38 + 36 + 28 + 18 + 17, 45 + 38 + 36 + 28 + 19 + 17, 45 + 38 + 36 + 28 + 20 + 17, 45 + 38 + 36 + 28 + 21 + 17,
45 + 38 + 36 + 28 + 22 + 17, 45 + 38 + 36 + 28 + 27 + 17, 45 + 38 + 37 + 28 + 17, 45 + 38 + 37 + 28 + 18 + 17, 45 + 38 + 37 + 28 + 19 + 17,
45 + 38 + 37 + 28 + 20 + 17, 45 + 38 + 37 + 28 + 21 + 17, 45 + 38 + 37 + 28 + 22 + 17, 45 + 38 + 37 + 28 + 27 + 17, 45 + 43 + 28 + 17,
45 + 43 + 28 + 18 + 17, 45 + 43 + 28 + 19 + 17, 45 + 43 + 28 + 20 + 17, 45 + 43 + 28 + 21 + 17, 45 + 43 + 28 + 22 + 17,
45 + 43 + 28 + 27 + 17, 45 + 43 + 29 + 28 + 17, 45 + 43 + 29 + 28 + 18 + 17, 45 + 43 + 29 + 28 + 19 + 17, 45 + 43 + 29 + 28 + 20 + 17,
45 + 43 + 29 + 28 + 21 + 17, 45 + 43 + 29 + 28 + 22 + 17, 45 + 43 + 29 + 28 + 27 + 17, 45 + 43 + 30 + 28 + 17, 45 + 43 + 30 + 28 + 18 + 17,
45 + 43 + 30 + 28 + 19 + 17, 45 + 43 + 30 + 28 + 20 + 17, 45 + 43 + 30 + 28 + 21 + 17, 45 + 43 + 30 + 28 + 22 + 17,
45 + 43 + 30 + 28 + 27 + 17, 45 + 43 + 31 + 28 + 17, 45 + 43 + 31 + 28 + 18 + 17, 45 + 43 + 31 + 28 + 19 + 17, 45 + 43 + 31 + 28 + 20 + 17,
45 + 43 + 31 + 28 + 21 + 17, 45 + 43 + 31 + 28 + 22 + 17, 45 + 43 + 31 + 28 + 27 + 17, 45 + 43 + 32 + 28 + 17, 45 + 43 + 32 + 28 + 18 + 17,
45 + 43 + 32 + 28 + 19 + 17, 45 + 43 + 32 + 28 + 20 + 17, 45 + 43 + 32 + 28 + 21 + 17, 45 + 43 + 32 + 28 + 22 + 17,
45 + 43 + 32 + 28 + 27 + 17, 45 + 43 + 33 + 28 + 17, 45 + 43 + 33 + 28 + 18 + 17, 45 + 43 + 33 + 28 + 19 + 17, 45 + 43 + 33 + 28 + 20 + 17,
45 + 43 + 33 + 28 + 21 + 17, 45 + 43 + 33 + 28 + 22 + 17, 45 + 43 + 33 + 28 + 27 + 17, 45 + 43 + 34 + 28 + 17, 45 + 43 + 34 + 28 + 18 + 17,
45 + 43 + 34 + 28 + 19 + 17, 45 + 43 + 34 + 28 + 20 + 17, 45 + 43 + 34 + 28 + 21 + 17, 45 + 43 + 34 + 28 + 22 + 17,
45 + 43 + 34 + 28 + 27 + 17, 45 + 43 + 35 + 28 + 17, 45 + 43 + 35 + 28 + 18 + 17, 45 + 43 + 35 + 28 + 19 + 17, 45 + 43 + 35 + 28 + 20 + 17,
45 + 43 + 35 + 28 + 21 + 17, 45 + 43 + 35 + 28 + 22 + 17, 45 + 43 + 35 + 28 + 27 + 17, 45 + 43 + 36 + 28 + 17, 45 + 43 + 36 + 28 + 18 + 17,
45 + 43 + 36 + 28 + 19 + 17, 45 + 43 + 36 + 28 + 20 + 17, 45 + 43 + 36 + 28 + 21 + 17, 45 + 43 + 36 + 28 + 22 + 17,
45 + 43 + 36 + 28 + 27 + 17, 45 + 43 + 37 + 28 + 17, 45 + 43 + 37 + 28 + 18 + 17, 45 + 43 + 37 + 28 + 19 + 17, 45 + 43 + 37 + 28 + 20 + 17,
45 + 43 + 37 + 28 + 21 + 17, 45 + 43 + 37 + 28 + 22 + 17, 45 + 43 + 37 + 28 + 27 + 17, 45 + 44 + 28 + 17, 45 + 44 + 28 + 18 + 17,
45 + 44 + 28 + 19 + 17, 45 + 44 + 28 + 20 + 17, 45 + 44 + 28 + 21 + 17, 45 + 44 + 28 + 22 + 17, 45 + 44 + 28 + 27 + 17,
45 + 44 + 29 + 28 + 17, 45 + 44 + 29 + 28 + 18 + 17, 45 + 44 + 29 + 28 + 19 + 17, 45 + 44 + 29 + 28 + 20 + 17, 45 + 44 + 29 + 28 + 21 + 17,
45 + 44 + 29 + 28 + 22 + 17, 45 + 44 + 29 + 28 + 27 + 17, 45 + 44 + 30 + 28 + 17, 45 + 44 + 30 + 28 + 18 + 17, 45 + 44 + 30 + 28 + 19 + 17,
45 + 44 + 30 + 28 + 20 + 17, 45 + 44 + 30 + 28 + 21 + 17, 45 + 44 + 30 + 28 + 22 + 17, 45 + 44 + 30 + 28 + 27 + 17, 45 + 44 + 31 + 28 + 17,
45 + 44 + 31 + 28 + 18 + 17, 45 + 44 + 31 + 28 + 19 + 17, 45 + 44 + 31 + 28 + 20 + 17, 45 + 44 + 31 + 28 + 21 + 17,
45 + 44 + 31 + 28 + 22 + 17, 45 + 44 + 31 + 28 + 27 + 17, 45 + 44 + 32 + 28 + 17, 45 + 44 + 32 + 28 + 18 + 17, 45 + 44 + 32 + 28 + 19 + 17,
45 + 44 + 32 + 28 + 20 + 17, 45 + 44 + 32 + 28 + 21 + 17, 45 + 44 + 32 + 28 + 22 + 17, 45 + 44 + 32 + 28 + 27 + 17, 45 + 44 + 33 + 28 + 17,
45 + 44 + 33 + 28 + 18 + 17, 45 + 44 + 33 + 28 + 19 + 17, 45 + 44 + 33 + 28 + 20 + 17, 45 + 44 + 33 + 28 + 21 + 17,
45 + 44 + 33 + 28 + 22 + 17, 45 + 44 + 33 + 28 + 27 + 17, 45 + 44 + 34 + 28 + 17, 45 + 44 + 34 + 28 + 18 + 17, 45 + 44 + 34 + 28 + 19 + 17,
45 + 44 + 34 + 28 + 20 + 17, 45 + 44 + 34 + 28 + 21 + 17, 45 + 44 + 34 + 28 + 22 + 17, 45 + 44 + 34 + 28 + 27 + 17, 45 + 44 + 35 + 28 + 17,
45 + 44 + 35 + 28 + 18 + 17, 45 + 44 + 35 + 28 + 19 + 17, 45 + 44 + 35 + 28 + 20 + 17, 45 + 44 + 35 + 28 + 21 + 17,
45 + 44 + 35 + 28 + 22 + 17, 45 + 44 + 35 + 28 + 27 + 17, 45 + 44 + 36 + 28 + 17, 45 + 44 + 36 + 28 + 18 + 17, 45 + 44 + 36 + 28 + 19 + 17,
45 + 44 + 36 + 28 + 20 + 17, 45 + 44 + 36 + 28 + 21 + 17, 45 + 44 + 36 + 28 + 22 + 17, 45 + 44 + 36 + 28 + 27 + 17, 45 + 44 + 37 + 28 + 17,
45 + 44 + 37 + 28 + 18 + 17, 45 + 44 + 37 + 28 + 19 + 17, 45 + 44 + 37 + 28 + 20 + 17, 45 + 44 + 37 + 28 + 21 + 17,
45 + 44 + 37 + 28 + 22 + 17, 45 + 44 + 37 + 28 + 27 + 17, 46 + 11, 46 + 12 + 11, 46 + 13 + 11, 46 + 14 + 11, 46 + 15 + 11,
46 + 15 + 12 + 11, 46 + 15 + 13 + 11, 46 + 15 + 14 + 11, 46 + 17, 46 + 18 + 17, 46 + 19 + 17, 46 + 20 + 17, 46 + 20 + 18 + 17,
46 + 20 + 19 + 17, 46 + 21 + 17, 46 + 22 + 17, 47 + 11, 47 + 12 + 11, 47 + 13 + 11, 47 + 14 + 11, 47 + 15 + 11, 47 + 15 + 12 + 11,
47 + 15 + 13 + 11, 47 + 15 + 14 + 11, 47 + 17, 47 + 18 + 17, 47 + 19 + 17, 47 + 20 + 17, 47 + 20 + 18 + 17, 47 + 20 + 19 + 17,
47 + 21 + 17, and 47 + 22 + 17;

in the list above the numbers refer to the embodiments according to their numbering provided hereinabove whereas “+” indicates the dependency from another embodiment. The different individualized embodiments are separated by commas. In other words, “4+2+1” for example refers to embodiment 4) depending on embodiment 2), depending on embodiment 1), i.e. embodiment “4+2+1” corresponds to the compounds of embodiment 1) further limited by the features of the embodiments 2) and 4).

It is to be understood that the range of i as described in embodiments 50) and 51) shall be regarded as explicitly disclosed for each of the above-listed combinations.

Where the plural form is used for compounds, conjugates, salts, pharmaceutical compositions, diseases or the like, this is intended to mean also a single compound, conjugate, salt, pharmaceutical composition, disease or the like.

Any reference to a compound of (I), (Ia), (II), (IIa), (IIb), (IIc), (III) and (IV) as defined in any one of embodiments 1) to 81) is to be understood as referring also to the salts (and especially the pharmaceutically acceptable salts) of such compounds, as appropriate and expedient.

The term “pharmaceutically acceptable salts” refers to salts that retain the desired biological activity of the subject compound and exhibit minimal undesired toxicological effects. Such salts include inorganic or organic acid and/or base addition salts depending on the presence of basic and/or acidic groups in the subject compound. They may also be used for stabilisation in the form of buffers or lyophilized products including buffer. For reference see for example ‘Handbook of Pharmaceutical Salts. Properties, Selection and Use.’, P. Heinrich Stahl, Camille G. Wermuth (Eds.), Wiley-VCH, 2008 and ‘Pharmaceutical Salts and Co-crystals’, Johan Wouters and Luc Quéré (Eds.), RSC Publishing, 2012.

The present embodiments also include isotopically labelled, especially ²H (deuterium) labelled compounds of Formula (I), (Ia), (II), (IIa), (IIb) and (IIc) which compounds are identical to the compounds of Formula (I), (Ia), (II), (IIa), (IIb) and (IIc) except that one or more atoms have each been replaced by an atom having the same atomic number but an atomic mass different from the atomic mass usually found in nature. Isotopically labelled, especially ²H (deuterium) labelled compounds of Formula (I), (Ia), (II), (IIa), (IIb) and (IIc), and salts thereof, are within the scope of the present embodiments. Substitution of hydrogen with the heavier isotope ²H (deuterium) may lead to greater metabolic stability, resulting e.g. in increased in-vivo half-life or reduced dosage requirements, or may lead to reduced inhibition of cytochrome P450 enzymes, resulting e.g. in an improved safety profile. In one embodiment, the compounds of Formula (I), (Ia), (II), (IIa), (IIb) and (IIc) are not isotopically labelled, or they are labelled only with one or more deuterium atoms. In a sub-embodiment, the compounds of Formula (I), (Ia), (II), (IIa), (IIb) and (IIc) are not isotopically labelled at all. Isotopically labelled compounds of Formula (I), (Ia), (II), (IIa), (IIb) and (IIc) may be prepared in analogy to the methods described hereinafter, but using the appropriate isotopic variation of suitable reagents or starting materials. For instance, the labelling may be performed within the linker L and/or spacer T.

The compounds of formula (I), (Ia), (II), (IIa), (IIb) and (IIc) as defined in any one of embodiments 1) to 55) and their pharmaceutically acceptable salts can be used as medicaments, e.g. in the form of pharmaceutical compositions for parenteral, enteral (such as oral) or nasal administration, in particular parenteral administration such as intramuscular, subcutaneous, and intradermal injections.

56) Hence, one aspect of the present invention relates to a pharmaceutical composition comprising, as active principle, an immunogenic compound according to any one of embodiments 1) to 55), in particular embodiments 52), 53), 54) and 55), or a pharmaceutically acceptable salt thereof, and at least one therapeutically inert excipient.

The production of the pharmaceutical compositions can be effected in a manner which will be familiar to any person skilled in the art (see for example Remington, The Science and Practice of Pharmacy, 23rd Edition (2021), published by Elsevier Inc., ISBN: 978-0-12-820007-0; Vaccine Development and Manufacturing, 1st edition (2014), published by John Wiley & Sons, ISBN:9780470261941) by bringing the described compounds of Formula (I), (Ia), (II), (IIa), (IIb) and (IIc) or their pharmaceutically acceptable salts, optionally in combination with other therapeutically valuable substances, into a galenical administration form together with suitable, non-toxic, inert, therapeutically compatible solid or liquid carrier materials and, optionally, usual pharmaceutical adjuvants.

The pharmaceutical composition may comprise in addition to an immunogenic compound according to any one of embodiments 1) to 55) one or more (preferably 1, 2, 3, or 4; more preferably 2, 3, or 4; and most preferably 2 or 3) other immunogenic compounds (oligosaccharide-carrier protein conjugates) that are immunogenic against one or more other K. pneumoniae serotypes, especially against O1, O2ac, O2aeh, O3, O3a, O3b, O4, O5, O7, O8, and/or O12; and notably against O1, O3, O3b and/or O5.

Said pharmaceutical composition is suitable for eliciting a protective immune response in a human and/or animal (especially a mammal (including a human)) host, and therefore is useful for the prevention and/or treatment of diseases associated with Klebsiella pneumoniae bacteria. Preferably, said pharmaceutical composition is suitable for use in human.

The terms “prevention”, “preventing” and/or “prophylaxis” are used synonymously and refer to inhibiting the initial onset of a pathologic process, such that the pathologic process that could eventually lead to development of symptoms never develops or that symptoms develop in lower, non-dangerous intensity (i.e. preventing the development of a disease, disorder, or condition in a prophylactic manner).

The present pharmaceutical composition is suitable for administration to animal (and, in particular, human) patients, and thus include both human and veterinary uses. It may be used in a method of raising an immune response in a patient, comprising the step of administering the composition to the patient.

The pharmaceutical compositions of the present invention may be administered before a subject is exposed to Klebsiella pneumoniae and/or after a subject is exposed to a Klebsiella pneumoniae. Preferably, it is used before a subject is exposed to Klebsiella pneumoniae.

Pharmaceutical compositions are preferably in aqueous form, particularly at the point of administration, but they can also be presented in non-aqueous liquid forms or in dried forms e.g. as gelatin capsules, or as lyophilisates, etc. Solid powders that are obtained e.g. by spray drying, spray-freeze drying, vacuum or air-drying, or lyophilisation, may be reconstituted before use. Lyophilisation is preferred in case solid powders shall be obtained.

The pharmaceutical composition may comprise one or more therapeutically inert excipients. Such excipient may be selected from the group consisting of citric acid monohydrate, sodium citrate, sodium citrate dihydrate, acetic acid, sodium hydroxide, tromethamine, tromethamine hydrochloride (to adjust pH), cholesterol, sorbitan trioleate, DSPC (1,2-distearoyl-sn-glycero-3-phosphocholine), and (4-hydroxybutyl)azanediyl) bis(hexane-6,1-diyl)bis(2-hexyldecanoate), polydimethylsiloxane (antifoam), ascorbic acid (antioxidant).

The excipient may serve to adjust tonicity, such as sodium chloride (NaCl), which may be present at from 1 to 20 mg/ml. Other salts that may be present include potassium chloride, potassium dihydrogen phosphate, disodium phosphate dehydrate, magnesium chloride, calcium chloride, etc.

The pharmaceutical composition may include one or more excipients which serve as preservatives which may be selected from the group consisting of 2-phenoxyethanol, benzethonium chloride, EDTA (ethylenediaminetetraacetic acid), formaldehyde, phenol and thiomersal (thimerosal). Mercury-free compositions are preferred, and preservative-free vaccines can be prepared.

The pharmaceutical composition may include one or more excipients which serve as surfactants which may be selected from the group consisting of polysorbate 20 (polyoxyethylene (20) sorbitan monolaurate), polysorbate 80 (polyoxyethylene (80) sorbitan monooleate), nonylphenol ethoxylate, octoxynol-10 and sodium deoxycholate.

The pharmaceutical composition may include compounds (with or without an insoluble metal salt) in plain water (e.g. water for injection, w.f.i.), but will usually include one or more buffers. Typical buffers include: a phosphate buffer; a Tris buffer; a borate buffer; a succinate buffer; a histidine buffer (particularly with an aluminum hydroxide adjuvant); or a citrate buffer. Buffer salts will typically be included in the 5-20 mM range.

Pharmaceutical compositions typically have a pH between 5.0 and 9.5 e.g. between 6.0 and 8.0.

The pharmaceutical composition may further include one or more stabilizer(s).

Pharmaceutical compositions are preferably sterile and gluten free.

57) A further embodiment of the present invention relates to the pharmaceutical composition according to embodiment 56), further comprising an adjuvant.

The term “adjuvant” as used herein refers to an immunological adjuvant i.e. a material used in a vaccine composition that modifies or augments the effects of said vaccine by enhancing the immune response to a given antigen contained in the vaccine without being antigenically related to it. For the person skilled in the art, classically recognized examples of immunological adjuvants include, but are not restricted to aluminum or calcium salt based adjuvants, saponins or saponin-based adjuvants (e.g. Matrix-M), CpG oligodexynucleotide based adjuvants (e.g. CpG 1018), oil-in-water emulsions (e.g. Freund's adjuvant, MF59), activators of natural killer T cells (NKT cells) or invariant NKT cells (e.g., glycosphingolipids such as KRN7000), toll-like receptor 1/2 (TLR-1/2) agonists (e.g., Pam3CSK4), TLR-3 agonists (e.g., Poly(I:C)), TLR-4 agonists (e.g., lipopolysaccharide), TLR-5 agonists (e.g., flagellin), TLR-7/8 agonists (e.g., resiquimod), immunomodulatory proteins (e.g., detoxified heat-labile enterotoxin (dmLT) from Escherichia coli), TLR-4 agonist glucopyranosyl lipid adjuvant-stable emulsion (GLA-SE) and monophosphoryl lipid A (MPL), non-ionic block polymers, cytokines (e.g., type 1 interferon (IFN), granulocyte-macrophage colony-stimulating factor (GM-CSF), interleukins), papain-like cysteine proteases, and many others such as e.g. AS04, AS03, AS01B, and formulations of the above mentioned adjuvants as liposomes or nanoparticles prepared with lipids such as DOPC (1,2-dioleoyl-sn-glycero-3-phosphocholine), DSPC (1,2-distrearoyl-sn-glycero-3-phosphocholine), cholesterol and/or ALC-0315, formulations as virus-like particles, and co-formulations of the abovementioned adjuvants, especially co-formulation including aluminum or calcium salt based adjuvants.

The adjuvant “aluminum”, “aluminum-based adjuvant” or “aluminum salt-based adjuvant” is one or more of the following: amorphous aluminum hydroxyphosphate sulfate (AAHS), aluminum hydroxide, aluminum phosphate, and potassium aluminum sulfate (Alum).

An example for a calcium-based or calcium salt-based adjuvant is calcium phosphate.

Matrix-M is a saponin-based adjuvant composed of nanoparticles from saponins extracted from Quillaja saponaria (soapbark) trees, cholesterol, and phospholipids.

CpG based adjuvants are immunostimulatory oligodeoxynucleotides bearing one or more CpG motifs (CpG ODN) that are unmethylated cytosine-guanine dinucleotides. The methylation status of the CpG immunostimulatory motif generally refers to the cytosine residue in the dinucleotide. An immunostimulatory oligonucleotide containing at least one unmethylated CpG dinucleotide is an oligonucleotide which contains a 5′ unmethylated cytosine linked by a phosphate bond to a 3′ guanine, and which activates the immune system through binding to Toll-like receptor 9 (TLR-9).

Freund's adjuvant is an oil-in-water adjuvant based on mineral oil.

MF59 is an oil-in-water emulsion comprising 4.3% w/v squalene, 0.5% w/v polysorbate 80 (Tween 80), and 0.5% w/v sorbitan trioleate (Span 85).

Glycosphingolipids are a class of lipids that stimulate unconventional invariant T-cell receptors on NKT cells or iNKT cells, when the glycosphingolipid is presented MHC class I-related molecules such as CD1d.

Pam3CSK4 (Pam3CysSerLys4) is a synthetic triacylated lipopeptide that is a ligand for TLR-1 and TLR-2. It mimics the acylated amino terminus of bacterial lipopeptides.

Poly(I:C) is a polymer and analogue of double-stranded RNA, consisting of one strand of a polymer of inosinic acid and one strand of a polymer of cytidylic acid. It stimulates TLR-3 and simulates viral infections.

Lipopolysaccharide (LPS) is a membrane component of Gram-negative bacteria and a stimulator of TLR-4.

Flagellin is a globular protein that forms the filaments of bacterial flagella. Flagellin activates TLR-5 and TLR-11.

Resiquimod (R848; 1-[4-Amino-2-(ethoxymethyl)-1H-imidazo[4,5-c]chinolin-1-yl]-2-methylpropan-2-ol) is an immune response modifier and small molecule that activates TLR-7 and TLR-8.

dmLT is the double-mutant (thereby detoxified) of heat-labile enterotoxin from Escherichia coli. It is an effective mucosal and systemic adjuvant.

GLA-SE is an oil-in-water emulsion adjuvant that is prepared by combining aqueous glucopyranosyl lipid A (GLA), a TLR-4 agonist, with squalene.

MPL (monophosphoryl lipid A), a truncated LPS, is a clinically used TLR-4 agonist.

Nonionic Block Polymers (NBPs) suitable as adjuvants are simple copolymers of polyoxyethylene (POE) and hydrophobic polyoxypropylene (POP) and differ in molecular weight, percentage POE and the mode of linkage of POE and POP-groups.

Cytokines are small proteins secreted by cells that affect the interaction and communication between cells. Typically, cytokines activate the target cell, leading to the secretion of additional cytokines and signaling cascades. Cytokines are involved in the induction of innate and adaptive immunity. As adjuvants, cytokines can be used as recombinant proteins or can be encoded on DNA molecules such as plasmids.

Papain-like cysteine proteases are derived from viruses, bacteria, yeast, protozoa, plants or animals and contain a cysteine thiol at the active site. This class of proteases can stimulate Th2 type immune responses.

AS04 (Adjuvant System 04) is a complex of MPL (3-O-desacyl-4′-monophosphoryl lipid A) and aluminum hydroxide or aluminum phosphate.

AS03 (Adjuvant System 03) is a squalene-in-water emulsion with DL-alpha-tocopherol (vitamin E) and polysorbate 80.

AS01B is a mixture of 3-O-desacyl-4′-monophosphoryl lipid A (MPL) and the saponin QS-21.

Preferred adjuvants are aluminum-based adjuvants, in particular aluminum hydroxide.

58) A further aspect of the present invention relates to the immunogenic compound according to any one of embodiments 1) to 55), in particular embodiments 52), 53), 54) and 55), or a pharmaceutically acceptable salt thereof, for the use as a medicament, in particular as a vaccine. In other words, the invention relates to a vaccine comprising the immunogenic compound according to any one of embodiments 1) to 55), in particular embodiments 52), 53), 54) and 55), or a pharmaceutically acceptable salt thereof. Preferably, the vaccine is used for active vaccination.

59) A further aspect of the present invention relates to an immunogenic compound according to any one of embodiments 1) to 55), in particular embodiments 52), 53), 54) and 55), or a pharmaceutically acceptable salt thereof, for the use in the prevention and/or treatment of a K. pneumoniae infection.

60) A further embodiment of the present invention relates to an immunogenic compound according to any one of embodiments 1) to 55), in particular embodiments 52), 53), 54) and 55), or a pharmaceutically acceptable salt thereof, for the use in the prevention and/or treatment of K. pneumoniae infections in individuals of 50 years or older; hospital acquired (i.e. nosocomial) K. pneumoniae infections, for instance nosocomial pneumonia, nosocomial bloodstream infections and nosocomial urinary tract infections; community-acquired K. pneumoniae infections; as well as pneumonia, bronchitis, meningitis, urinary tract infection, intra-abdominal infections, wound infection, infection of blood, osteomyelitis, bacteremia, septicemia, liver abscess and inflammatory bowel disease (IBD), all caused by K. pneumoniae infection.

A population-based strategy for vaccination of individuals of 50 years or older against K. pneumoniae infections is desirable, because this population is particularly susceptible to K. pneumoniae infections, in particular individuals of 60 years or older and at risk of exposure to K. pneumoniae and/or anticipated weakened immune system.

K. pneumoniae is a notorious pathogen frequently responsible for hospital acquired (i.e. nosocomial) respiratory and urinary tract infections. It is the second most common cause of Gram-negative bacteremia. Drug resistant isolates are associated with high mortality (greater than 50% according to some studies), add significantly to hospital stays, and are especially problematic in ICUs.

Therefore, it is desired to prevent hospital acquired (i.e. nosocomial) K. pneumoniae infections, in particular in populations at high risk of exposure, including patients who will undergo elective surgery with hospital stays longer than 72 hours (e.g., joint replacements), patients with weakened immune systems and patients who anticipate having weakened immune systems (e.g., those on solid organ transplant wait lists, non-urgent solid tumor surgery followed by chemotherapy). In these populations, vaccination 2-8 weeks prior to surgery, optionally followed by a booster may be applicable.

Moreover, the prevention of community-acquired infections in specific target groups such as healthcare workers or elderly (60 years or older) in long-term care facilities or nursing homes is desired. The term “community-acquired K. pneumoniae infections” relates to any K. pneumoniae infection acquired in the community. In contrast to a nosocomial (hospital-acquired) infection.

Furthermore, the present immunogenic compound according to any one of embodiments 1) to 55), in particular embodiments 52), 53), 54) and 55), or a pharmaceutically acceptable salt thereof, may be used in the prevention and/or treatment of pneumonia, bronchitis, meningitis, urinary tract infection, intra-abdominal infections, wound infection, infection of blood, osteomyelitis, bacteremia, septicemia, liver abscess, and inflammatory bowel disease (IBD,) all caused by K. pneumoniae infection.

61) A further embodiment of the present invention relates to an immunogenic compound according to any one of embodiments 1) to 55), in particular embodiments 52), 53), 54) and 55), or a pharmaceutically acceptable salt thereof, for the use in the prevention and/or treatment of the K. pneumoniae infections as listed in embodiments 59) and 60) above, wherein K. pneumoniae is selected from 0-serotypes comprising O2a and O2afg.

62) For any avoidance of doubt, the immunogenic compound according to any one of embodiments 1) to 55), in particular embodiments 52), 53), 54) and 55), or a pharmaceutically acceptable salt thereof, as well as the pharmaceutical composition of embodiment 56) or 57), and the vaccine according to embodiment 58) are likewise suitable for the prevention and/or the treatment of the K. pneumoniae infections as listed in any one of embodiments 59), 60) and 61).

63) Preferably, the immunogenic compound according to any one of embodiments 1) to 55), in particular embodiments 52), 53), 54) and 55), or a pharmaceutically acceptable salt thereof, as well as the pharmaceutical composition of embodiment 56) or 57), and the vaccine according to embodiment 58) are suitable for the prevention or prophylaxis of the K. pneumoniae infections as listed in any one of embodiments 59), 60) and 61).

64) A further aspect of the present invention relates to a method of eliciting an immune response against K. pneumoniae in a human and/or animal (especially a mammal (including a human)) host, comprising administering to the human and/or animal an effective amount of the immunogenic compound according to any one of embodiments 1) to 55), in particular embodiments 52), 53), 54) and 55), or a pharmaceutically acceptable salt thereof. The administered amount is preferably from 0.05 μg to 30 μg glycan per immunization of the human patient. The term “glycan” refers to antigen, i.e. oligosaccharide excluding linker L and spacer T. Possibly, more than one immunization is required.

65) Likewise, an embodiment of the present invention relates to a method of eliciting an immune response against K. pneumoniae in a human and/or animal (especially a mammal (including a human)) host, comprising administering to the human and/or animal an effective amount of the composition according to embodiment 56) or 57), as well as the vaccine according to embodiment 58).

66) For avoidance of any doubt, if immunogenic compounds according to any one of embodiments 1) to 55), in particular embodiments 52), 53), 54) and 55), or pharmaceutically acceptable salts thereof, are described as useful for the prevention and/or treatment of a K. pneumoniae infection according to any one of embodiments 59), 60) and 61), such immunogenic compounds are likewise suitable for use in the preparation of a medicament for the prevention and/or treatment of said K. pneumoniae infection according to any one of embodiments 59), 60) and 61).

67) A further aspect of the present invention relates to a multivalent vaccine comprising the immunogenic compound according to any one of embodiments 1) to 55), preferably the immunogenic compound according embodiments 52), 53), 54) and 55), or a pharmaceutically acceptable salt thereof.

The term “multivalent vaccine” in this respect relates to a vaccine comprising antigens against two or more different K. pneumoniae strains, in particular to two or more pathogenic K. pneumoniae strains.

68) A further aspect of the present invention relates to an intermediate compound for preparing the immunogenic compound according to any one of embodiments 28) to 55), having the formula (III)

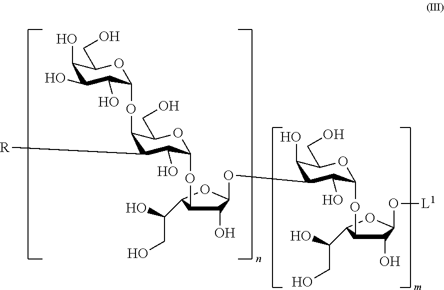

wherein
R is OH, or

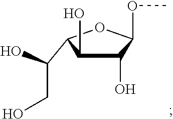

m is 3, 4, 5, 6, 7 or 8; preferably 3, 4, 5 or 6;
n is 1, 2, 3, 4, 5 or 6; preferably 2, 3, or 4;
L¹ represents
   *—(C₂₋₁₀)alkylene-NH₂;
   *—(CH₂CH₂O)_{b}—CH₂CH₂NH₂, wherein b is 1, 2 or 3;
   *—CH₂CH₂S—CH₂CH₂NH₂;
   *—(C₂₋₁₀)fluoroalkylene-NH₂;
   *—(CH₂)_{c}NHC(O)(CH₂)_{c′}—NH₂, wherein c and c′ are independently from each other from 2 to 6;
   *—(CH₂)_{d}NHC(O)NH(CH₂)_{d′}—NH₂, wherein d and d′ are independently from each other from 2 to 6;
   *—(C₁₋₁₀)alkylene-C(O)—NH—(C₂₋₁₀)alkylene-NH₂;
   *—(C₂₋₁₀)alkylene-O—NH₂; or
   *—(C₂₋₁₀)alkylene-SH;
or a pharmaceutically acceptable salt thereof.

69) A further embodiment relates to the intermediate compound according to embodiment 68), or a pharmaceutically acceptable salt thereof, wherein
m is 4, n is 2 and R is OH;
m is 4, n is 2 and R is

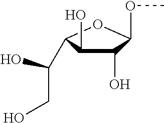

or;
m is 4, n is 3 and R is OH.

70) A further embodiment relates to the intermediate compound according to embodiment 68) or 69), or a pharmaceutically acceptable salt thereof, wherein
L¹ represents
   *—(CH₂)ₐ—NH₂; wherein a is from 2 to 10;
   *—(CH₂CH₂O)_{b}—CH₂CH₂NH₂, wherein b is 1, 2 or 3;
   *—CH₂CH₂S—CH₂CH₂NH₂;
   *—(C₂₋₁₀)fluoroalkylene-NH₂ with fluoroalkylene being a saturated straight chain;
   *—(CH₂)_{c}NHC(O)(CH₂)_{c′}—NH₂, wherein c and c′ are independently from each other from 2 to 6;
   *—(CH₂)_{d}NHC(O)NH(CH₂)_{d′}—NH₂, wherein d and d′ are independently from each other from 2 to 6;
   *—(CH₂)ₑ—C(O)—NH—(CH₂)_{e′}—NH₂; wherein e is from 1 to 10 and e′ is from 2 to 10;
   *—(CH₂)_{f}—O—NH₂, wherein f is from 2 to 10; or
   *—(CH₂)_{g}—SH, wherein g is from 2 to 10.

It is to be understood that in an analogous manner, embodiments 30) to 37) disclose further preferred L¹ which bear terminal amino- or SH-groups as demonstrated in embodiment 68).

71) A further embodiment relates to the intermediate compound according to embodiment 68) or 69), or a pharmaceutically acceptable salt thereof, wherein
L¹ represents
   *—(CH₂)₂—NH₂, *—(CH₂)₃—NH₂, *—(CH₂)₄—NH₂, *—(CH₂)₅—NH₂, or *—(CH₂)₆—NH₂; preferably *—(CH₂)₅—NH₂.

72) A further embodiment relates to the intermediate compound according to embodiment 69), or a pharmaceutically acceptable salt thereof, wherein
m is 4, n is 2 and R is OH; and
L¹ represents
   *—(CH₂)₂—NH₂, *—(CH₂)₃—NH₂, *—(CH₂)₄—NH₂, *—(CH₂)₅—NH₂, or *—(CH₂)₆—NH₂; preferably *—(CH₂)₅—NH₂.

73) A further aspect of the present invention relates to an intermediate compound for preparing the immunogenic compound according to any one of embodiments 28) to 55), or a pharmaceutically acceptable salt thereof, having the formula (IV):

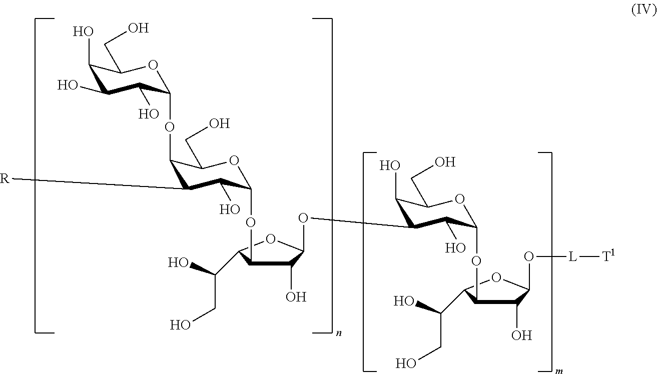

wherein
R is OH, or

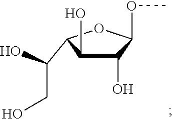

m is 3, 4, 5, 6, 7 or 8; preferably 3, 4, 5 or 6;
n is 1, 2, 3, 4, 5 or 6; preferably 2, 3, or 4;
L represents
   *—(C₂₋₁₀)alkylene-NH—;
   *—(CH₂CH₂O)_{b}—CH₂CH₂NH—, wherein b is 1, 2 or 3;
   *—CH₂CH₂S—CH₂CH₂NH—;
   *—(C₂₋₁₀)fluoroalkylene-NH—;
   *—(CH₂)_{c}NHC(O)(CH₂)_{c′}—NH—, wherein c and c′ are independently from each other from 2 to 6;
   *—(CH₂)_{d}NHC(O)NH(CH₂)_{d′}—NH—, wherein d and d′ are independently from each other from 2 to 6;
   *—(C₁₋₁₀)alkylene-C(O)—NH—(C₂₋₁₀)alkylene-NH—; or
   *—(C₂₋₁₀)alkylene-O—NH—; and
T¹ represents
   —C(O)—(C₀₋₁₀)alkylene-C(O)X;
   —C(O)—CH₂CH₂—(OCH₂CH₂)ⱼ—C(O)X, wherein j is from 1 to 5;
   —C(O)—CH₂(CH₂)ₖ—(SCH₂(CH₂)_{k′})_{k″}—C(O)X, wherein k is 0 or 1, k′ is 0 or 1, and k″ is 1, 2, or 3;

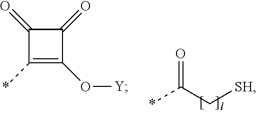

wherein l is 1 or 2; or

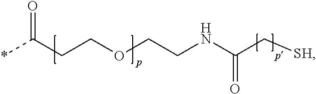

wherein p is from 1 to 4, preferably 1, and p′ is 1 or 2;
—C(O)X represents —C(O)OH or an activated ester; and
Y represents Me, Et, Bu or —(CH₂CH₂O)₃CH₃ (especially Me, Et, n-Bu or —(CH₂CH₂O)₃CH₃).

The term “activated ester” refers to a functionalized carboxylic acid with enhanced reactivity towards amines (in comparison to a carboxylic acid), for the reaction with the amino group of a lysine residue of CRM₁₉₇.

74) A further embodiment relates to the intermediate compound according to embodiment 73), or a pharmaceutically acceptable salt thereof, wherein
m is 4, n is 2 and R is OH;
m is 4, n is 2 and R is

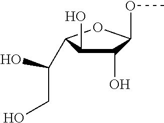

or;
m is 4, n is 3 and R is OH.

75) A further embodiment relates to the intermediate compound according to embodiment 73) or 74), or a pharmaceutically acceptable salt thereof, wherein
L represents
   *—(CH₂)ₐ—NH—; wherein a is from 2 to 10;
   *—(CH₂CH₂O)_{b}—CH₂CH₂NH—, wherein b is 1, 2 or 3;
   *—CH₂CH₂S—CH₂CH₂NH—;
   *—(C₂₋₁₀)fluoroalkylene-NH— with fluoroalkylene being a saturated straight chain;
   *—(CH₂)_{c}NHC(O)(CH₂)_{c′}—NH—, wherein c and c′ are independently from each other from 2 to 6;
   *—(CH₂)_{d}NHC(O)NH(CH₂)_{d′}—NH—, wherein d and d′ are independently from each other from 2 to 6;
   *—(CH₂)ₑ—C(O)—NH—(CH₂)_{e′}—NH—; wherein e is from 1 to 10 and e′ is from 2 to 10; or
   *—(CH₂)_{f}—O—NH—, wherein f is from 2 to 10; and
T¹ represents
   —C(O)—(CH₂)ₕ—C(O)X, wherein h is from 0 to 10;
   —C(O)—CH₂CH₂—(OCH₂CH₂)ⱼ—C(O)X, wherein j is from 1 to 5;
   —C(O)—CH₂(CH₂)ₖ—(SCH₂(CH₂)_{k′})_{k″}—C(O)X, wherein k is 0 or 1, k′ is 0 or 1, and k″ is 1, 2, or 3;

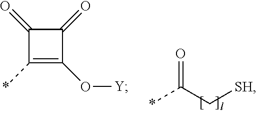

wherein l is 1 or 2; or

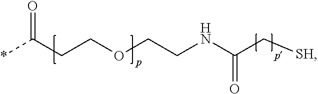

wherein p is from 1 to 4, preferably 1, and p′ is 1 or 2;
—C(O)X represents —C(O)OH or an activated ester; and
Y represents Me, Et, Bu or —(CH₂CH₂O)₃CH₃ (especially Me, Et, n-Bu or —(CH₂CH₂O)₃CH₃).

76) A further embodiment relates to the intermediate compound according to embodiment 73) or 74), or a pharmaceutically acceptable salt thereof, wherein
L represents
   *—(CH₂)₂—NH—, *—(CH₂)₃—NH—, *—(CH₂)₄—NH—, *—(CH₂)₅—NH—, or *—(CH₂)₆—NH—; preferably *—(CH₂)₅—NH—;
T¹ represents
   —C(O)—C(O)X, —C(O)—CH₂—C(O)X, —C(O)—(CH₂)₂—C(O)X, —C(O)—(CH₂)₃—C(O)X, —C(O)—(CH₂)₄—C(O)X, —C(O)—(CH₂)₅—C(O)X, or —C(O)—(CH₂)₆—C(O)X;
   preferably —C(O)—(CH₂)₄—C(O)X; and
—C(O)X represents —C(O)OH or an activated ester.

77) A further embodiment relates to the intermediate compound according to embodiment 76), or a pharmaceutically acceptable salt thereof, wherein
m is 4, n is 2 and R is OH;
L represents
   *—(CH₂)₂—NH—, *—(CH₂)₃—NH—, *—(CH₂)₄—NH—, *—(CH₂)₅—NH—, or *—(CH₂)₆—NH—;
   preferably *—(CH₂)₅—NH—;
T¹ represents
   —C(O)—C(O)X, —C(O)—CH₂—C(O)X, —C(O)—(CH₂)₂—C(O)X, —C(O)—(CH₂)₃—C(O)X, —C(O)—(CH₂)₄—C(O)X, —C(O)—(CH₂)₅—C(O)X, or —C(O)—(CH₂)₆—C(O)X;
preferably —C(O)—(CH₂)₄—C(O)X; and
—C(O)X represents —C(O)OH or an activated ester.

In embodiments 73), 74), 75), 76) and 77), preferably, X represents

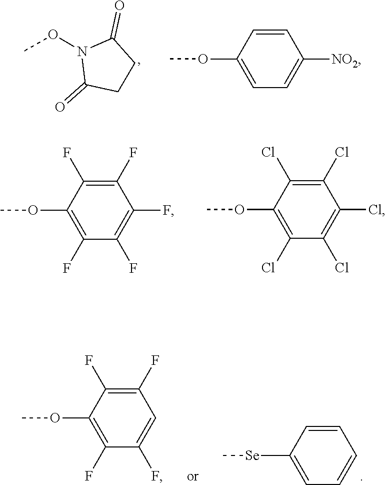

It is to be understood that embodiments 30) to 37) disclose further preferred L which are encompassed in the present embodiment. Moreover, it is to be understood that in an analogous manner, embodiments 39) to 44) disclose further preferred T, which translate to T¹, wherein the terminal “C(O)—” is replaced by “C(O)X”, and in case of squaric acid, the attachment point to the carrier protein, e.g. to CRM₁₉₇, is denoted as “O—Y”, and R¹ is H. These preferred T¹ are to be regarded as explicitly disclosed.

78) A further aspect of the present invention relates to an immuno-assay comprising the oligosaccharide hybrid antigen of formula (I)

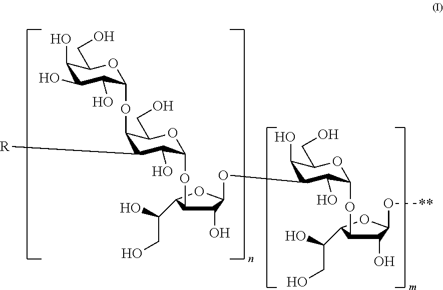

wherein
R is OH, or

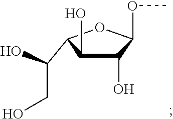

m is 3, 4, 5, 6, 7 or 8; and
n is 1, 2, 3, 4, 5 or 6.

In this embodiment, the “**” in the dotted line relates to the point of attachment to an array surface, preferably via a linker and/or spacer. The oligosaccharide hybrid antigen of formula (I) can be attached to any carrier surface suitable for arrays or micro-arrays, either with or without a linker and/or spacer.

79) A preferred embodiment of the present invention relates to the immuno-assay according to embodiment 78), comprising the compound of formula (Ia):

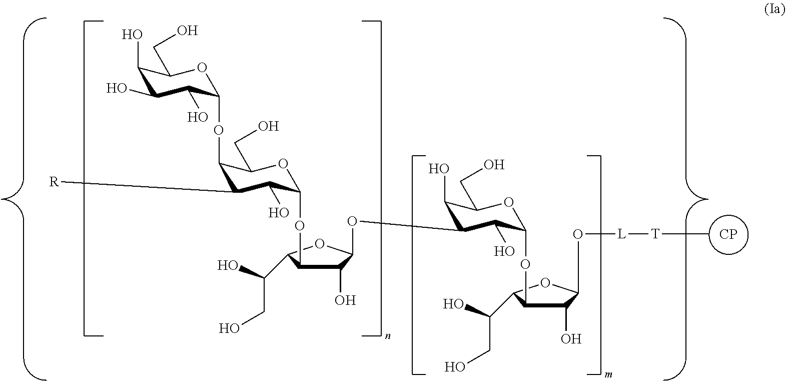

wherein
R is OH, or

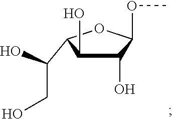

m is 3, 4, 5, 6, 7 or 8; preferably 3, 4, 5 or 6; and
n is 1, 2, 3, 4, 5 or 6; preferably 2, 3, or 4;
i is at least 1, preferably from 1 up to a number corresponding to 90% of the count of lysine residues contained in the carrier protein CP;
-L-T- represents a linker L and a spacer T as disclosed in any one of embodiments 17) or 23) to 47); and CP is a carrier protein suitable for immunological assays, in particular ELISA. Preferred CP are described in embodiments 8) and 9). A particularly preferred carrier protein is BSA.

The synthesis of compounds of the antigen of formula (Ia) conjugated to BSA are described and exemplified in the experimental part. It is to be understood that this synthesis applies analogously to all antigens of formula (Ia) so that the skilled person can prepare them.

The assay of this embodiment is suitable for the detection of antibodies against K. pneumoniae O2a and O2afg strains.

80) A further aspect of the present invention relates to the immunogenic compound according to any one of embodiments 28) to 55), or a pharmaceutically acceptable salt thereof, wherein the immunogenic compound is obtainable by or prepared by conjugating the compound of formula (IV):

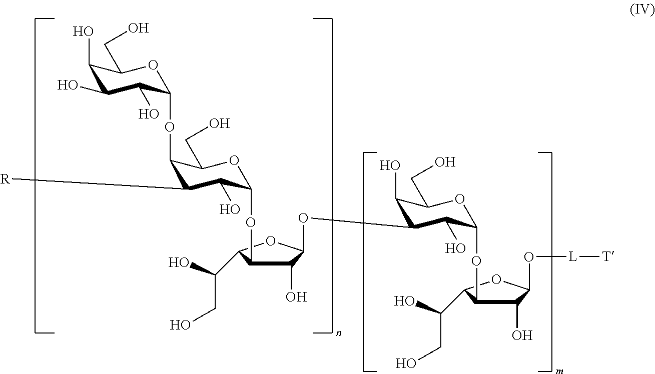

wherein
R is OH, or

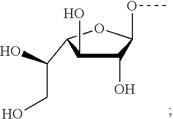

m is 3, 4, 5, 6, 7 or 8; preferably 3, 4, 5 or 6;
n is 1, 2, 3, 4, 5 or 6; preferably 2, 3, or 4;
L represents
   *—(C₂₋₁₀₀)alkylene-NH—;
   *—(CH₂CH₂O)_{b}—CH₂CH₂NH—, wherein b is 1, 2 or 3;
   *—CH₂CH₂S—CH₂CH₂NH—;
   *—(C₂₋₁₀)fluoroalkylene-NH—;
   *—(CH₂)_{c}NHC(O)(CH₂)_{c′}—NH—, wherein c and c′ are independently from each other from 2 to 6;
   *—(CH₂)_{d}NHC(O)NH(CH₂)_{d′}—NH—, wherein d and d′ are independently from each other from 2 to 6;
   *—(C₁₋₁₀)alkylene-C(O)—NH—(C₂₋₁₀)alkylene-NH—; or
   *—(C₂₋₁₀)alkylene-O—NH—; and
a) T¹ represents
   —C(O)—(C₀₋₁₀)alkylene-C(O)X;
   —C(O)—CH₂CH₂—(OCH₂CH₂)ⱼ—C(O)X, wherein j is from 1 to 5;
   —C(O)—CH₂(CH₂)ₖ—(SCH₂(CH₂)_{k′})_{k″}—C(O)X, wherein k is 0 or 1, k′ is 0 or 1, and k″ is 1, 2, or 3;

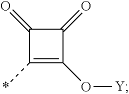

—C(O)X represents —C(O)OH or an activated ester; and
Y represents Me, Et, Bu or —(CH₂CH₂O)₃CH₃
to a lysine residue of CRM₁₉₇; or
b) conjugating the compound of formula (IV) wherein T¹ represents

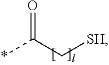

wherein l is 1 or 2; or

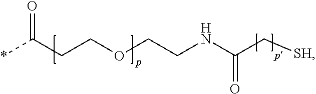

wherein p is from 1 to 4, preferably 1, and p′ is 1 or 2; or
wherein L-T¹ represents
   *—(C₂₋₁₀)alkylene-SH;
to modified CRM₁₉₇ selected from the group consisting of:

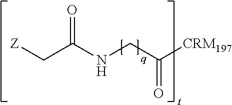

wherein Z is Br or I, q is 2 or 3,
   and t is from 1 to 28;

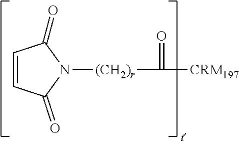

wherein r is 2 or 3, and t′ is from 1 to 28; and

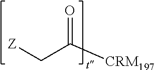

wherein Z is Br or I, and t″ is from 1 to 28.

Preferably, X represents

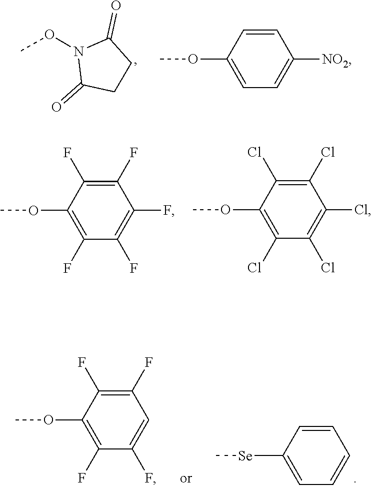

It is to be understood that embodiments 29) to 37) disclose further preferred L, and embodiments 73) and 75) to 77) disclose further preferred T¹ which are all encompassed in the present embodiment. Moreover, it is to be understood that in an analogous manner, embodiments 39) to 44) disclose further preferred T, which translate to T¹, wherein the terminal “C(O)—” is replaced by “C(O)X”, and in case of squaric acid, the attachment point to the carrier protein, e.g. to CRM₁₉₇, is denoted as “O—Y”, and R¹ is H. These preferred T¹ are to be regarded as explicitly disclosed.

81) A further aspect of the present invention relates to a process for preparing the immunogenic compound according to any one of embodiments 28) to 55), or a pharmaceutically acceptable salt thereof, wherein the process comprises conjugating the compound of formula (IV):

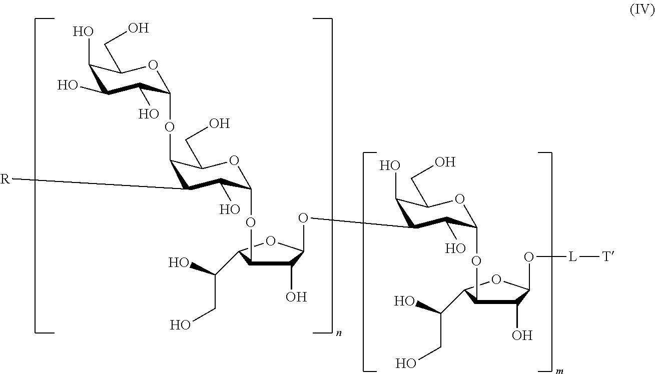

wherein
R is OH, or

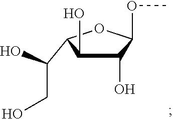

m is 3, 4, 5, 6, 7 or 8; preferably 3, 4, 5 or 6;
n is 1, 2, 3, 4, 5 or 6; preferably 2, 3, or 4;
L represents
   *—(C₂₋₁₀₀)alkylene-NH—;
   *—(CH₂CH₂O)_{b}—CH₂CH₂NH—, wherein b is 1, 2 or 3;
   *—CH₂CH₂S—CH₂CH₂NH—;
   *—(C₂₋₁₀)fluoroalkylene-NH—;
   *—(CH₂)_{c}NHC(O)(CH₂)_{c′}—NH—, wherein c and c′ are independently from each other from 2 to 6;
   *—(CH₂)_{d}NHC(O)NH(CH₂)_{d′}—NH—, wherein d and d′ are independently from each other from 2 to 6;
   *—(C₁₋₁₀)alkylene-C(O)—NH—(C₂₋₁₀)alkylene-NH—; or
   *—(C₂₋₁₀)alkylene-O—NH—; and
a) T represents
   —C(O)—(C₀₋₁₀)alkylene-C(O)X;
   —C(O)—CH₂CH₂—(OCH₂CH₂)ⱼ—C(O)X wherein j is from 1 to 5;
   —C(O)—CH₂(CH₂)ₖ—(SCH₂(CH₂)_{k′})_{k″}—C(O)X, wherein k is 0 or 1, k′ is 0 or 1, and k″ is
   1, 2, or 3;

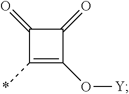

—C(O)X represents an activated ester;
Y represents Me, Et, Bu or —(CH₂CH₂O)₃CH₃;
to a lysine residue of CRM₁₉₇; or
b) conjugating the compound of formula (IV) wherein T¹ represents

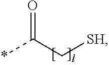

wherein l is 1 or 2; or

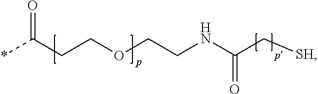

wherein p is from 1 to 4, preferably 1, and p′ is 1 or 2; or
wherein L-T¹ represents
   *—(C₂₋₁₀)alkylene-SH;
to modified CRM₁₉₇ selected from the group consisting of:

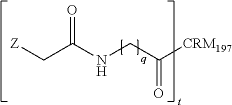

wherein Z is Br or I, q is 2 or 3, and
   t is from 1 to 28;

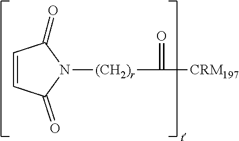

wherein r is 2 or 3, and t′ is from 1 to 28; and

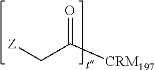

wherein Z is Br or I, and t″ is from 1 to 28.

Preferably, X represents

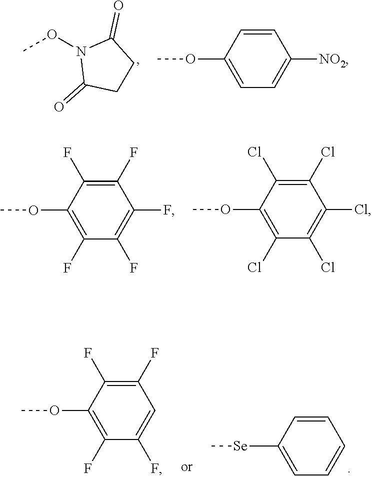

It is to be understood that embodiments 29) to 37) disclose further preferred L, and embodiments 73) and 75) to 77) disclose further preferred T¹ which are all encompassed in the present embodiment. Moreover, it is to be understood that in an analogous manner, embodiments 39) to 44) disclose further preferred T, which translate to T¹, wherein the terminal “C(O)—” is replaced by “C(O)X”, and in case of squaric acid, the attachment point to the carrier protein, e.g. to CRM₁₉₇, is denoted as “O—Y”, and R¹ is H. These preferred T¹ are to be regarded as explicitly disclosed.

Whenever the word “between” or “to” is used to describe a numerical range, it is to be understood that the end points of the indicated range are explicitly disclosed and included in the range. For example: if a temperature range is described to be between 40° C. and 80° C. (or 40° C. to 80° C.), this means that the end points 40° C. and 80° C. are included in the range; or if a variable is defined as being an integer between 1 and 4 (or 1 to 4), this means that the variable is the integer 1, 2, 3, or 4.

However, for the avoidance of any doubt, the definition “a bridge having a backbone with a length of 5 to 25 atoms covalently linked together that forms the shortest distance between the oxygen at C1 of the reducing end of the oligosaccharide and the nitrogen of the amino group of a lysine residue at the carrier protein CRM₁₉₇” means that the oxygen at C1 and the nitrogen of the amino group of the lysine at the CRM₁₉₇ do not count to the numbering of the so-defined backbone.

Unless used regarding temperatures, the term “about” (or alternatively “around”) placed before a numerical value “X” refers in the current application to an interval extending from X minus 10% of X to X plus 10% of X, and preferably to an interval extending from X minus 5% of X to X plus 5% of X. In the particular case of temperatures, the term “about” (or alternatively “around”) placed before a temperature “Y” refers in the current application to an interval extending from the temperature Y minus 10° C. to Y plus 10° C., and preferably to an interval extending from Y minus 5° C. to Y plus 5° C. Besides, the term “room temperature” as used herein refers to a temperature of about 25° C.

### Preparation of Compounds of Formula (I), (La), (II), (IIa), (IIb), (IIc), (III) and (IV)

A further aspect of the invention is a process for the preparation of compounds of Formula (I), (Ia), (II), (IIa), (IIb), (IIc), (III) and (IV). Compounds according to Formula (I), (Ia), (II), (IIa), (IIb), (IIc), (III) and (IV) of the present invention can be prepared from commercially available or well-known starting materials according to the methods described in the experimental part; by analogous methods; or according to the general sequence of reactions outlined below, wherein L, T, L¹, T¹, X and Y are as defined for Formula (I), (Ia), (II), (IIa), (IIb), (IIc), (III) and (IV). Other abbreviations used herein are explicitly defined, or are as defined in the experimental section.

The synthesis of the compounds of the present invention requires protection group strategy. Though such protecting group strategy may be sophisticated, the use of protecting groups is well known in the art (see for example “Protective Groups in Organic Synthesis”, T. W. Greene, P. G. M. Wuts, Wiley-Interscience, 1999). The compounds obtained may also be converted into salts, especially pharmaceutically acceptable salts thereof in a manner known per se.

### General Preparation Routes:

### Antigen Representation

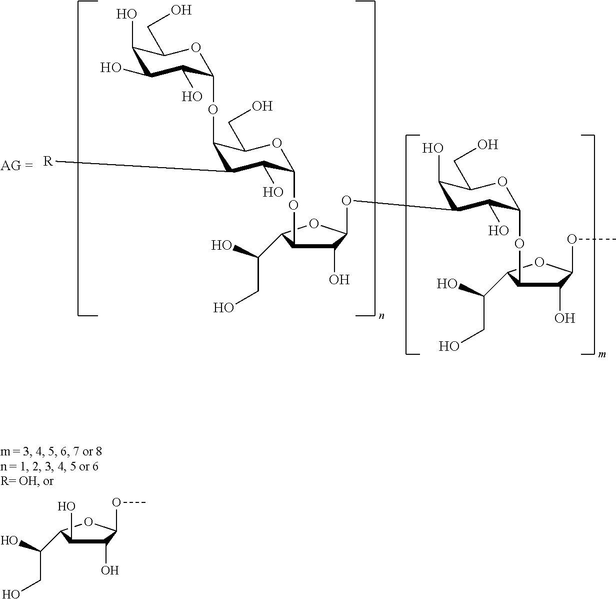

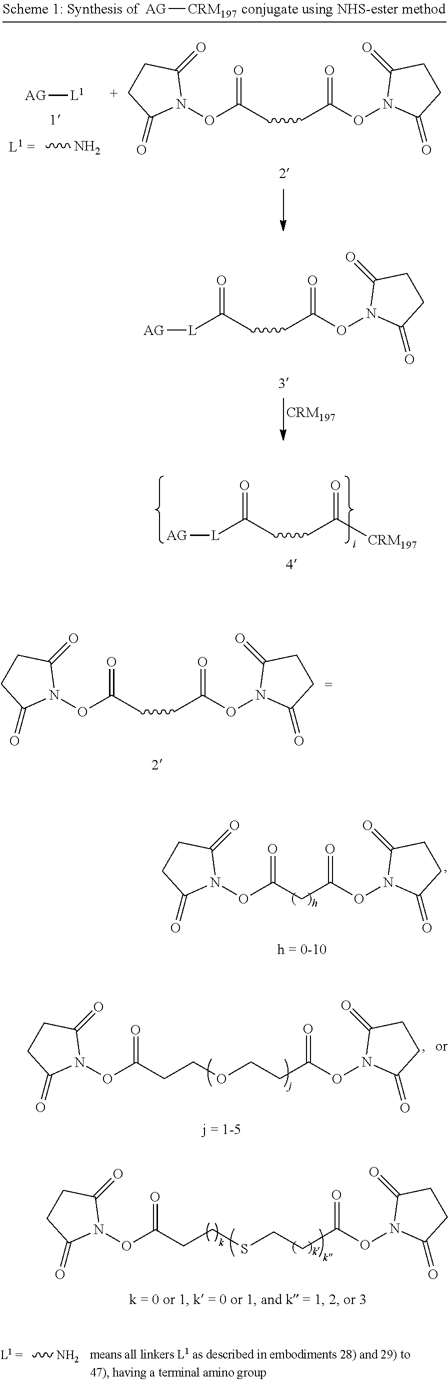

Antigen AG-L¹ 1′ in appropriate solvent (e.g., DMSO) in a vial at rt is treated with activated Bis-NHS ester of the diacid 2′ (e.g., Bis-NHS adipate, which is commercially available or can be prepared by the person skilled in the art using corresponding Bis-acid and N-hydroxy succinic acid) (Odom, O. W., Biochemistry, Vol. 29, No. 48, 1990) (5-20 equiv.) in DMSO in presence of triethylamine and stirred for 3 h at rt. The Antigen-NHS ester 3′ is precipitated out by adding EtOAc, and centrifuged, subsequently the precipitate is washed with EtOAc, dried in vacuum before taken for the next step. The buffer solution containing Antigen-NHS ester 3′ (25-100 equiv.) and CRM₁₉₇ is stirred at rt for 20-24 h. The resulting Antigen-CRM₁₉₇ conjugate 4′ is washed, purified and stored using appropriate buffer solution.

In the above synthesis routes, CRM₁₉₇ may be replaced by any one of the carrier proteins as described in embodiments 8) or 9).

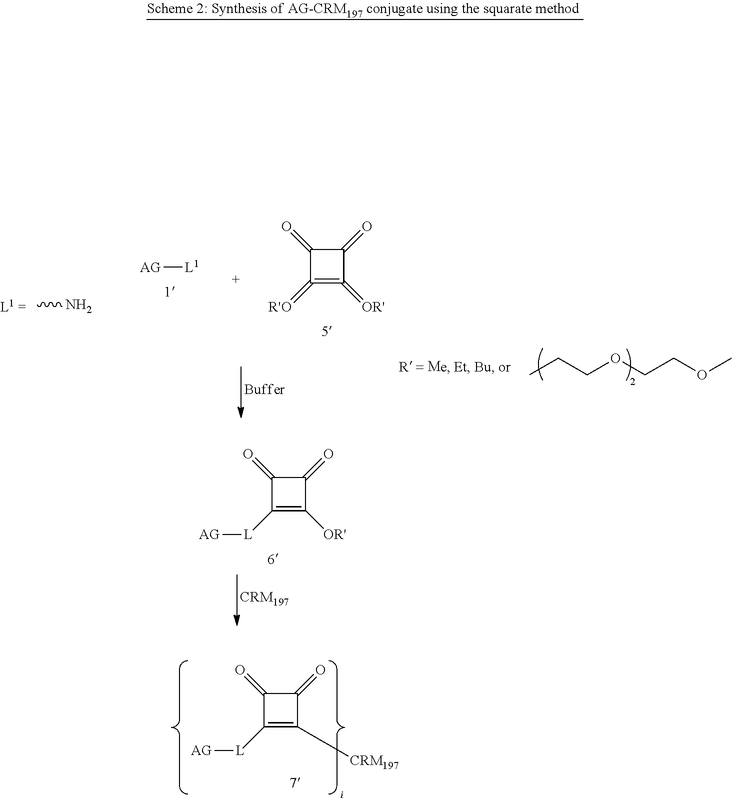

Antigen AG-L¹ 1′ in appropriate solvent (e.g., H₂O-EtOH, buffer) in a vial at rt is treated with desired alkyl squarate 5′ (e.g., 3,4-dibutoxy-3-cyclobutene-1,2-dione, 3,4-(Di(2-(2-(2-methoxyethoxy)ethoxy)ethoxy)-3-cyclobutene-1,2-dione) (Ganesh et al, JACS, 2014, 136, 16260-16269 and Xu et al, Carbhydr. Res, 2018, 456, 24-29) and stirred at rt in solvent with appropriate pH (7-8). The reaction mixture is neutralized using acetic acid and then concentrated (or lyophilized) in vacuum. The crude is purified using C18 (or SEC) column using water-acetonitrile as eluents. The fractions containing product are frozen and lyophilized to afford 6′. The 0.5 M pH 9 borate buffer solution containing Antigen-squarate ester 6′ (25-100 equiv.) and CRM₁₉₇ is stirred at rt for 24-72 h (S. Hou et al, Carbhydr. Res, 2008, 343, 196-210). The resulting Antigen-CRM₁₉₇ conjugate 7′ is washed, purified, and stored using appropriate buffer solution.

In the above synthesis routes, CRM₁₉₇ may be replaced by any one of the carrier proteins as described in embodiments 8) or 9).

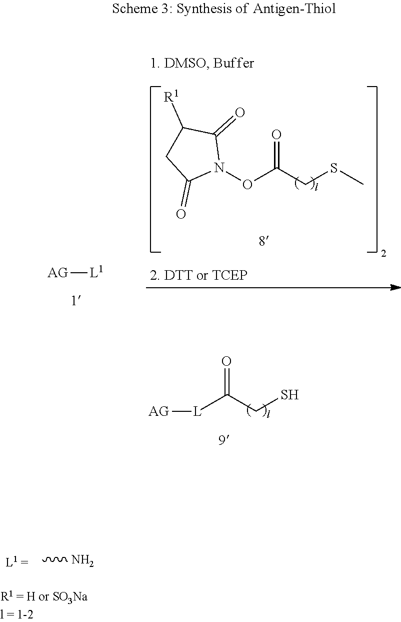

Antigen AG-L¹ 1′ in appropriate solvent (e.g., DMSO) in a vial at rt is treated with 8′ (e.g., DSP (dithiobis(succinimidylpropionate), or DTSSP (3,3′-dithiobis(sulfo-succinimidylpropionate)), to obtain the corresponding disulfide, which in turn is reduced by DTT (dithiothreitol) or TCEP (tris(2-carboxyethyl)phosphine) to afford the Antigen-thiol 9′.

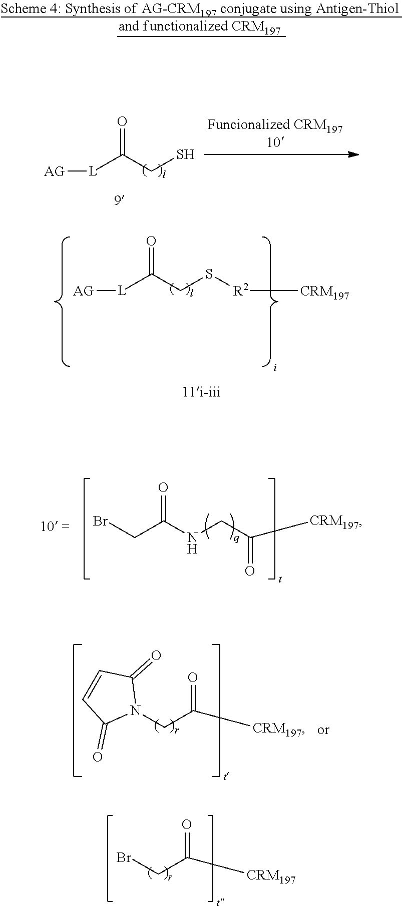

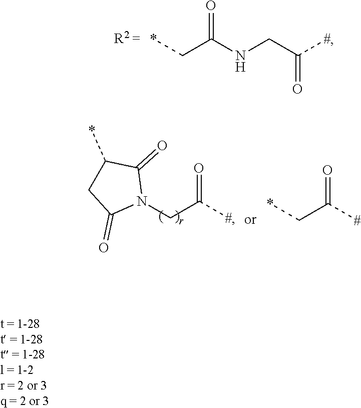

### a) Synthesis of AG-CRM₁₉₇ Conjugate Using Antigen-Thiol-Maleimide Method

The buffer solution containing Antigen-thiol 9′ (25-100 equiv.) and CRM₁₉₇ functionalized with Maleimide 10′ (which can for instance be prepared by treating CRM₁₉₇ with 3-maleimido-propionic acid succinimidyl ester or any other appropriate NHS ester equipped with maleimide, by person skilled in the art) (Robert M. F. van der Put et al, ACS Cent. Sci. 2022, 8, 4, 449-460) is stirred at rt for 20-24 h. Then excess maleimide moieties are quenched by adding L-cysteine in buffer to the RM and stirring for an hour at rt. The resulting Antigen-CRM₁₉₇-thio-maleimide conjugate 11′ is washed, purified, and stored using appropriate buffer solution.

In the above synthesis routes, CRM₁₉₇ may be replaced by any one of the carrier proteins as described in embodiments 8) or 9).

### b) Synthesis of AG-CRM₁₉₇ Conjugate Using Antigen-Thiol-Ether Method

The buffer solution containing Antigen-thiol 9′ (25-100 equiv.) and protein functionalized with α-bromoacetate 10′ (e.g., CRM₁₉₇-BAP, synthesized using CRM₁₉₇ and SBAP (N-succinimidyl 3-(2-bromoacetamido)propanoate) or any other appropriate NHS ester equipped with α-bromoacetate) (Schumann, B. et al, Chem. Sci., 2014, 5, 1992-2002) is stirred at rt for 24 h. Then excess α-bromoacetate moieties are quenched by adding L-cysteine in buffer to the RM and stirring for an hour at rt. The resulting Antigen-CRM₁₉₇-thio-ether conjugate 11′ is washed, purified, and stored using appropriate buffer solution.

In the above synthesis routes, CRM₁₉₇ may be replaced by any one of the carrier proteins as described in embodiments 8) or 9).

### General Retro Synthetic Approach to the AG-Hybrid-Antigen RS-1

The Antigen RS-1 can be synthesized as shown in scheme 5 using functionalized building blocks. The completely deprotected antigen RS-1 is equipped with a linker L1 at its reducing end which is essential for the conjugation with the protein carriers. The linker L1 is as disclosed in embodiments 28) and 29) to 47). RS-1 can be accessed from deprotection of the completely protected RS-2. The deprotection strategies may include removal of esters, amide, imide, carbamate via (acidic or basic) hydrolysis, hydrogenolysis, birch reduction, reduction of azide group to amine. The deprotection sequence depends on the protecting groups and their compatibility with reaction conditions. A person skilled in the art is able to accomplish this successfully. RS-2 can be obtained from glycosylation of RS-3 as a donor and RS-4 as an acceptor. RS-4 can be accessed from intermediates RS-5 and RS-6, which is equipped with the appropriate linker handle (Lx). RS-9 donor can be treated with linker handle of the choice from various linker handles listed in (Table A) to get RS-6. RS-5 synthesized using RS-7 and RS-8, and RS-9 can be accessed from the repeating unit RS-8 as well. So, the common intermediate RS-8 can be obtained from monosaccharide building blocks RS-10 and RS-11.

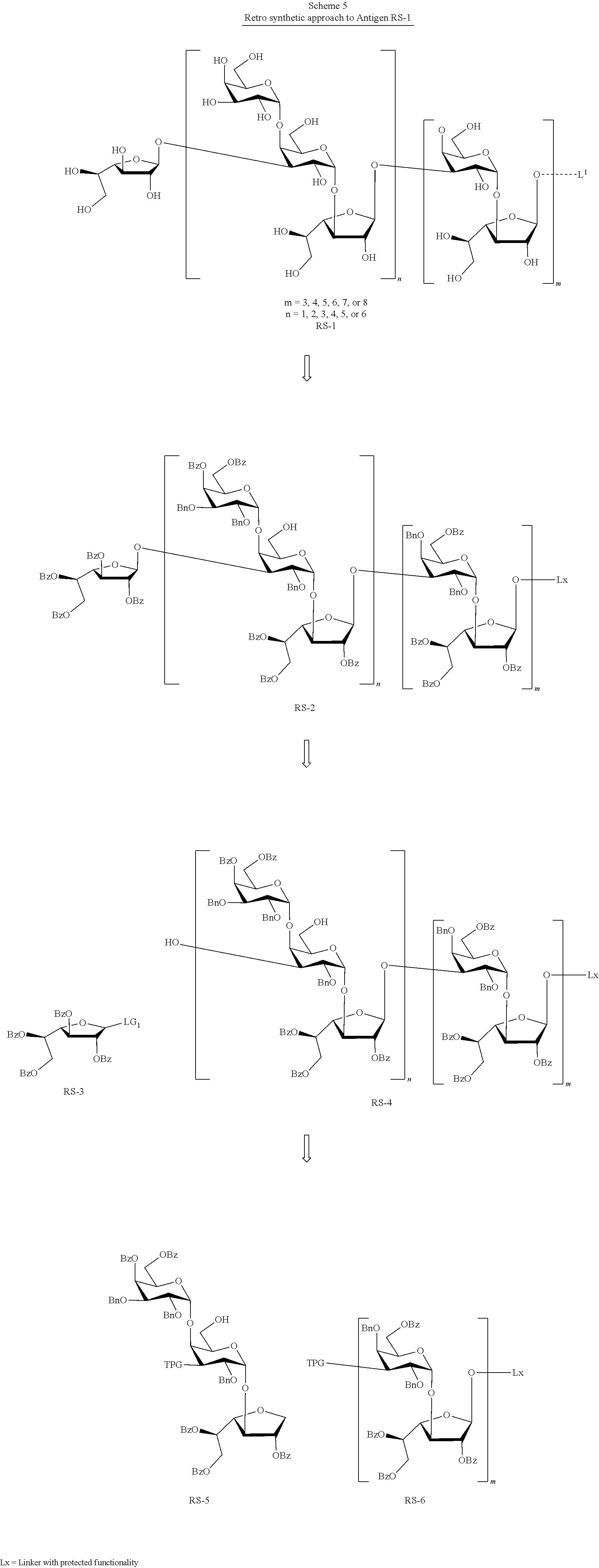

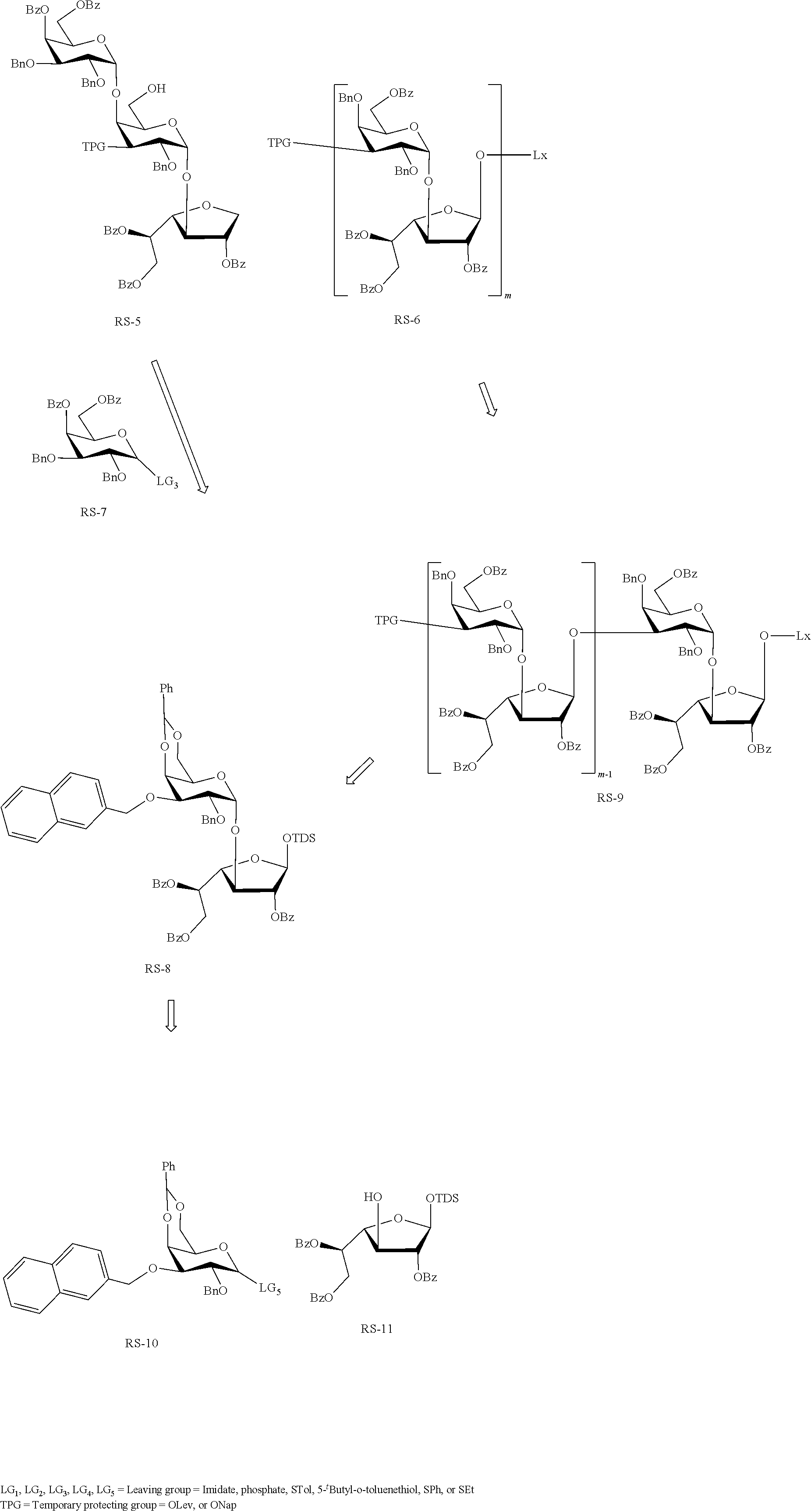

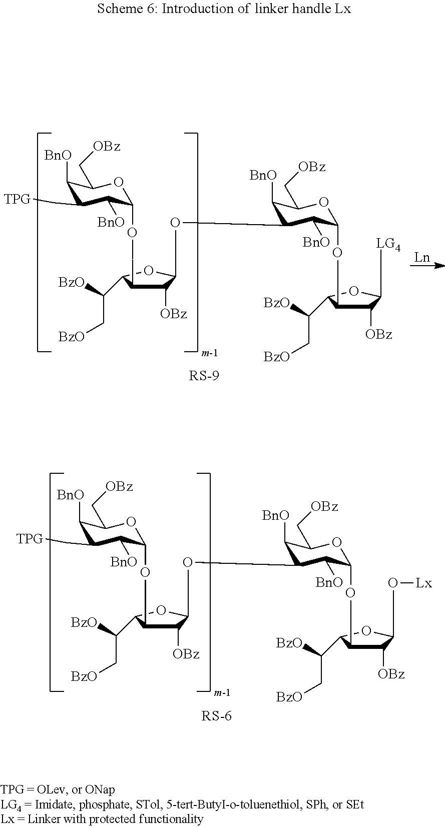

Linker nucleophile Ln (e.g., 5-azidopentan-1-ol) and the RS-9 donor are taken in RBF and dried azeotropically using dry toluene in the vacuum. The mixture is taken in appropriate solvent (e.g., DCM) at rt, 4 A molecular sieves are added to it and stirred for 30-45 min under N₂ atmosphere. The RM is cooled to appropriate temperature (e.g., 0° C. to −20° C.) and an activator (e.g., TMSOTf, TfOH) is added to the RM and stirred the RM for 20 mins. The RM is then allowed to warm slowly to room temp over one hr. Reaction completion is monitored by TLC. The RM is quenched (e.g., with sat. NaHCO₃, Na₂S₂O₃ solution), and extracted with solvent (e.g., DCM, EtOAc). The combined organics are washed with water, brine, dried, evaporated in vacuum to get crude. The crude product is purified by silica column chromatography using EA/cyclohexane as eluents. Fractions containing product are evaporated and dried in vacuum to get product RS-6.

TABLE A
List of nucleophilic linkers Ln

HO—(C₂₋₁₀)alkylene-N₃ or HO—(C₂₋₁₀)alkylene-NBnCbz;
HO—(CH₂CH₂O)_{b}—CH₂CH₂—N₃ or HO—(CH₂CH₂O)_{b}—CH₂CH₂—NBnCbz, wherein b is 1,
2 or 3;
HO—CH₂CH₂S—CH₂CH₂N₃ or HO—CH₂CH₂S—CH₂CH₂NBnCbz;
HO—(C₂₋₁₀)fluoroalkylene-N₃ or HO—(C₂₋₁₀)fluroalkylene-NBnCbz;
HO—(CH₂)_{c}NHC(O)(CH₂)_{c′}—N₃ or HO—(CH₂)_{c}NHC(O)(CH₂)_{c′}—NBnCbz, wherein c and
c′ are independently from each other from 2 to 6;
HO—(CH₂)_{d}NHC(O)NH(CH₂)_{d′}—N₃ or HO—(CH₂)_{d}NHC(O)NH(CH₂)_{d′}— NBnCbz, wherein
d and d′ are independently from each other from 2 to 6;
HO—(C₁₋₁₀)alkylene-C(O)—NH—(C₂₋₁₀)alkylene-N₃ or
HO—(C₁₋₁₀)alkylene-C(O)—NH—(C₂₋₁₀)alkylene-NBnCbz
HO—(C₁₋₁₀)alkylene-C(O)—OR where R is alkyl or benzyl;
HO—(C₂₋₁₀)alkylene-O—NH—^{t}Boc; or HO—(C₂₋₁₀)alkylene-O—N-pthalimide;
HO—(C₂₋₁₀)alkylene-S—Ac

### EXPERIMENTAL SECTION

### Abbreviations (as Used Herein and in the Description Above)

AcOH Acetic acid
aq. Aqueous
Bn Benzyl
BSA Bovine serum albumin
CDCl₃ Deuterated chloroform
Cs₂CO₃ Cesium carbonate
Cy Cyclohexane
D₂O Deuterium oxide
DCM Dichloromethane
DDQ 2,3-dichloro-5,6-dicyano-1,4-benzoquinone
DMAP 4-(Dimethylamino)pyridine
DMF N, N-dimethylformamide
DMSO Dimethylsulfoxide
ELISA Enzyme-linked immunosorbent assay
equiv Equivalents
ESI Electrospray ionization
Et₃N (TEA) Triethylamine
EtOAc (EA) Ethyl acetate
EtOH Ethanol
EtSH Ethanethiol
Fr Fraction
h Hours
H₂ Hydrogen
H₂O Water
H₂SO₄ Sulfuric acid
HCl Hydrochloric acid
HPLC High-performance liquid chromatography
HPLC-SEC High-performance liquid chromatography-size exclusion
chromatography
I₂ Iodine
ICU Intensive Care Unit
IPA Isopropanol
LPS Lipopolysaccharide
M Molar
MeOH Methanol
Min Minutes
MS Molecular sieves
N₂ Nitrogen
Na Sodium
Na₂S₂O₃ Sodium thiosulfate
Na₂SO₄ Sodium sulfate
NaCl Sodium chloride
NaHCO₃Sodium bicarbonate
NaOMe Sodium methoxide
NaPi buffer Sodium phosphate buffer
NH₂NH₂ Hydrazine
NIS N-iodosuccinimide
NMR Nuclear magnetic resonance spectroscopy
PBS Phosphate-buffered saline
PBS-T Phosphate-buffered saline with 0.1% (v/v) Tween-20
Pd(OH)₂ Palladium hydroxide
Pd/C Palladium on carbon
py Pyridine
RBF Round bottom flask
RM Reaction Mixture
rt Room temperature
sat. Saturated
SDS-PAGE Sodium dodecyl sulfate-polyacrylamide gel electrophoresis
SM Starting material
sol. Solution
TBAF Tetrabutylammonium fluoride
TBS Tris-buffered saline
TDS Dimethyl-Thexylsilylchloride
TLC Thin layer chromatography
TMB 3,3′,5,5′-Tetramethylbenzidine
TMSOTf Trimethylsilyl trifluoromethanesulfonate
UV Ultraviolet

### I. Chemistry

The following examples illustrate the preparation of biologically active compounds of the invention but do not at all limit the scope thereof.

### General Information:

All reagents and solvents were used as purchased and solvents used for the reactions were anhydrous. Except reactions containing water as solvent, all reactions were conducted under an atmosphere of N₂ in dried glassware (purchased from VWR and ROTH). Before glycosylation it is highly recommended to dry acceptor and donor by azeotrope with anhydrous toluene twice. Heidolph magnetic stirrer was used to carry out the experiments. Thin-layer chromatography (TLC) was performed on silica gel 60 F254 glass plates (Merck) or aluminium plates (VWR). Developed TLC plates were visualized under a short-wave UV lamp and by heating plates that were dipped in sugar stain solution (3-methoxy phenol (0.225 mL), H₂SO₄ (6 mL) and EtOH (200 mL)). All automated flash chromatography purifications on silica gel (FlashPure Silica 40 μm irregular: BUCHI columns) were carried out with Biotage Isolera and Biotage Select. BUCHI rotary evaporator was used to evaporate the solvent. Dry ice and acetone and ice/water combination were used for cooling the reaction mixture to get the desired temperatures. All NMR experiments were carried out on BRUKER 400 MHz instrument.

Temperatures are indicated in degrees Celsius (° C.). In mixtures, relations of parts of solvent or eluent or reagent mixtures in liquid form are given as volume relations (v/v), unless indicated otherwise.

### Characterisation Methods Used:

HPLC-SEC: The glycoconjugates used for immunizations were analyzed by HPLC-SEC to observe mass differences between conjugated and unconjugated CRM₁₉₇ proteins. The samples were diluted in 50 mM Tris, 20 mM NaCl, pH 7.2 and run on an Agilent 1100 HPLC system fitted with Tosoh TSK G2000 column (SWxl, 7.8 mm×30 cm, 5 μm) and a Tosoh TSK gel Guard column (SWxl 6.0 mm×4 cm, 7 μm). The flow rate was kept at 1 mL/min.

SDS-PAGE: The samples were diluted in Laemmli loading buffer and heated for 5 min at 95° C. After cooling at RT for 5 min, approximately 2-2.5 μg of the samples were loaded into the wells of a 10% polyacrylamide gel along with approx. 5 μL of the protein size marker. The samples were run at a constant voltage of 120 V for approximately 30-45 min.

Staining was done using the Gel Code™ Blue Safe Protein Stain as per manufacturer's instructions. The gels were washed with deionized water overnight and scanned.

### Synthesis of Diol Disaccharide D1:

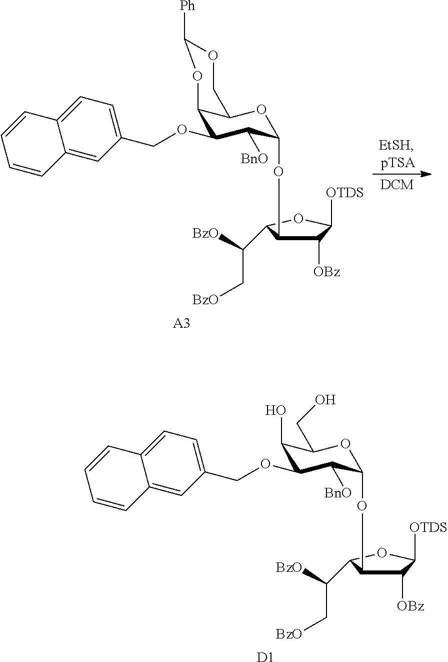

To a solution of the disaccharide A3 ((see WO2019106201, page 164), 68 g, 61 mmol) in HPLC grade DCM (305 mL) was added EtSH (27.1 mL, 366 mmol) and TsOH·H₂O (3.15 g, 18.29 mmol). After stirring at room temperature for 40 min, TLC analysis (EtOAC/Hexanes, 1/1) showed the disappearance of the starting material and the presence of a new spot. Then, the reaction mixture was quenched with triethylamine (2.55 mL, 18.29 mmol) and concentrated under reduced pressure. The residue was purified by flash silica gel column chromatography (gradient DCM/MeOH, 0 to 10%). The fractions containing the product were concentrated in vacuo and dried under high vacuum to afford D1 as a colorless oil (55 g, 88%). HRMS calcd for C₅₉H₇₀NO₁₄Si⁺ [M+NH₄]⁺ 1044.4560, found 1044.460.

### Synthesis of Disaccharide Acceptor D2:

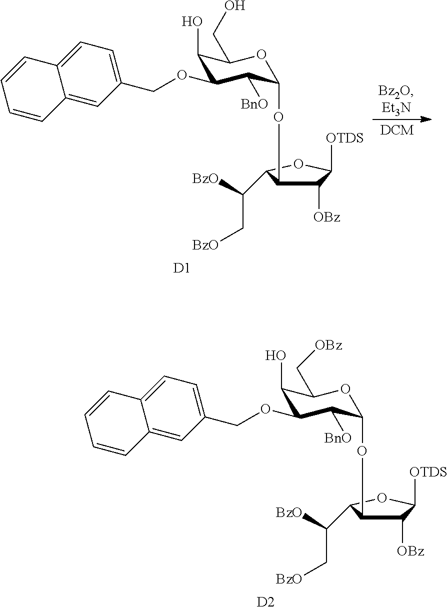

Benzoic anhydride (12.51 g, 55.3 mmol) and triethylamine (38.9 g, 53.6 mL) were added to a solution of the diol D1 (49.4 g, 48.1 mmol) in anhydrous DCM (385 mL) and the reaction let stir overnight at room temperature. The reaction mixture was transferred to a separatory funnel and washed with sat. aq. sol. NaHCO₃ (150 mL). The layers were separated and the aqueous layer extracted with DCM (150 mL). The combined organic layers were dried over Na₂SO₄ and the solvent concentrated in rotavapor. The residue was purified using automated purification system (Cy/EtOAc, gradient 0 to 100%). The tubes containing the product were combined and the solvent evaporated to give the product D2 as a white foam (48.5 g, 89%). HRMS calcd for C₆₆H₇₄NO₁₅Si⁺ [M+NH₄]⁺ 1148.4822, found 1148.487.

### Synthesis of Protected Trisaccharide D3:

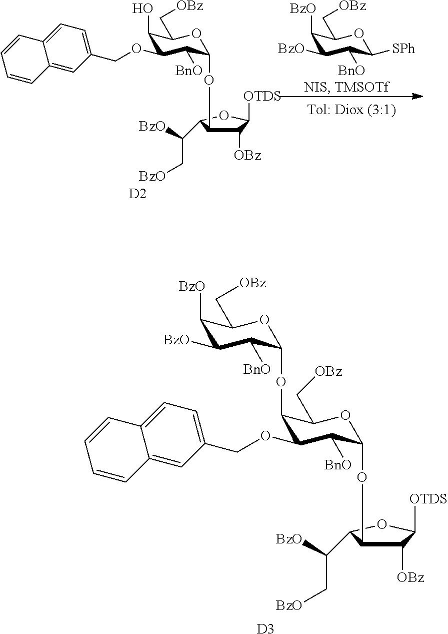

To a solution of the Phenyl 4,6-di-O-benzoyl-2,3-di-O-benzyl-1-thio-β-D-galactopyranoside donor (25.5 g, 38.6 mmol) and acceptor D2 (32 g, 28.3 mmol) in toluene:dioxane (3:1, 515 mL) was added freshly activated 4 Å MS and the mixture let stir at room temperature for 45 min. Then, NIS (10.18 g, 45.3 mmol) was added and the reaction mixture cooled to 0° C. TMSTOf (0.51 mL, 2.83 mmol) was added and the reaction mixture stirred for 1.5 h at 0° C. The reaction was filtered, quenched with NaHCO₃ (150 mL), diluted with ethyl acetate (150 mL) and extracted with 0.1 M aq. sol. Na₂S₂O₃, sat. aq. sol. NaHCO₃ and brine. The organic layer was dried over Na₂SO₄ and the solvent concentrated in rotavapor. Purification by automated purification system (Cy/EtOAc, gradient 0 to 100%) afforded the product D3 after evaporation of the solvent as a white foam (43.8 g, 92%). HRMS calcd for C₁₀₀H₁₀₄NO₂₂Si⁺ [M+NH₄]⁺ 1698.6814, found 1699.695.

### Synthesis of Trisaccharide Alcohol D4:

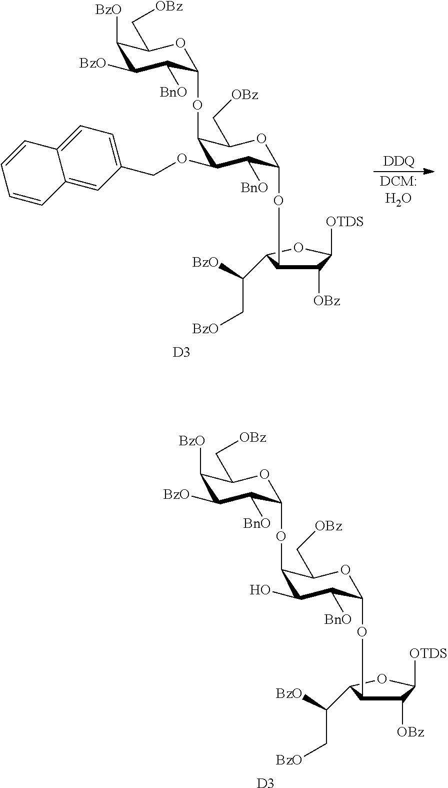

To a solution of the NAP protected trisaccharide D3 (60 g, 35.7 mmol) in DCM:MeOH (9:1, 350 mL) in a 500 mL RBF, was added DDQ (10.12 g, 44.6 mmol) at 0° C. The reaction mixture was warmed to room temperature and stirred for 2.5 h. Reaction was monitored by TLC (EtOAc in Cy, 3:1). Reaction was diluted with DCM (100 mL) and quenched with sat. aq. sol. NaHCO₃ (100 mL). The organic layer was washed with NaHCO₃ (2×100 mL) and brine (100 mL). The organic layer was dried over Na₂SO₄, filtered, and the filtrate was concentrated under vacuum to obtain the crude product. The crude product was purified by automated flash chromatography (Cy/EtOAc, gradient 0 to 100%). Concentration of solvent gave the product D4 as a white foam (43.5 g, 79%). HRMS calcd for C₈₉H₉₆NO₂₂Si⁺ [M+NH₄]⁺ 1558.6188, found 1559.633.

### Synthesis of Thexyldimethylsilyl 4,6-di-O-benzoyl-2,3-di-O-benzyl-α-D-galactopyranosyl-(1→4)-6-O-benzoyl-2-O-benzyl-3-O-levulinoyl-α-D-galactopyranosyl-(1→3)-2,5,6-tri-O-benzoyl-β-D-galactofuranoside D5

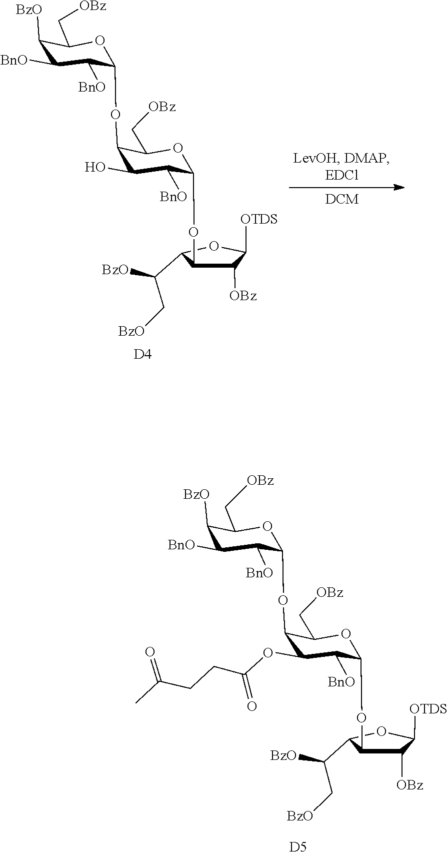

The trisaccharide starting material D4 (30 g, 19.46 mmol) was dissolved in anhydrous DCM (195 mL) and LevOH (9.04 g, 78 mmol), EDCI (14.92 g, 78 mmol) and DMAP (3.82 g, 68.1 mmol) were added sequently. The reaction mixture was stirred at room temperature and monitored by TLC. After 72 h, the mixture was partitioned between DCM and brine. The organic layers were dried over Na₂SO₄, filtered and evaporated to give the crude product. The crude was charged on isolute and purified using automated purification system with Cy/EtOAc (0-100%) to give the product D5 as a white foam (28.4 g, 89%). HRMS calcd for C₉₄H₁₀₂NO₂₄Si⁺ [M+NH₄]⁺ 1656.6556, found 1657.664. ¹H NMR (400 MHz, CDCl₃) δ 7.92-7.86 (m, 4H), 7.86-7.76 (m, 6H), 7.67-7.62 (m, 2H), 7.45-7.14 (m, 20H), 7.11-6.90 (m, 13H), 5.80 (d, J=2.0 Hz, 1H), 5.74-5.67 (m, 1H), 5.33 (s, 1H), 5.21 (dd, J=10.8, 2.6 Hz, 1H), 5.18 (s, 1H), 5.12 (d, J=3.7 Hz, 1H), 4.78-4.71 (m, 2H), 4.57-4.29 (m, 13H), 4.14 (d, J=2.4 Hz, 1H), 4.10 (d, J=5.5 Hz, 1H), 4.07-4.00 (m, 1H), 3.99-3.94 (m, 1H), 3.82 (dd, J=10.7, 3.5 Hz, 1H), 3.74 (dd, J=10.1, 3.2 Hz, 1H), 2.63-2.53 (m, 2H), 2.53-2.39 (m, 2H), 2.00 (s, 3H), 1.51-1.45 (m, 1H), 0.71 (dd, J=6.8, 0.9 Hz, 6H), 0.68 (s, 6H), 0.03 (s, 3H), 0.00 (s, 3H). ¹³C NMR (101 MHz, CDCl₃) δ 206.3, 172.5, 166.3, 166.1, 166.1, 165.8, 165.8, 165.3, 138.2, 138.1, 138.0, 133.3, 133.2, 133.1, 133.0, 130.1, 130.0, 129.9, 129.9, 129.8, 129.7, 129.6, 129.4, 128.8, 128.6, 128.5, 128.5, 128.5, 128.5, 128.4, 128.4, 128.4, 128.3, 128.1, 128.0, 127.9, 127.7, 127.5, 101.1, 101.0, 99.7, 85.1, 83.5, 81.8, 77.4, 76.5, 74.5, 73.8, 73.3, 72.8, 72.1, 71.7, 70.7, 69.4, 68.4, 67.8, 63.9, 62.7, 62.1, 38.0, 34.2, 29.9, 28.3, 24.9, 20.2, 20.0, 18.7, 18.6, −2.2, −3.3.

### Synthesis of Trisaccharide Hemiacetal D6:

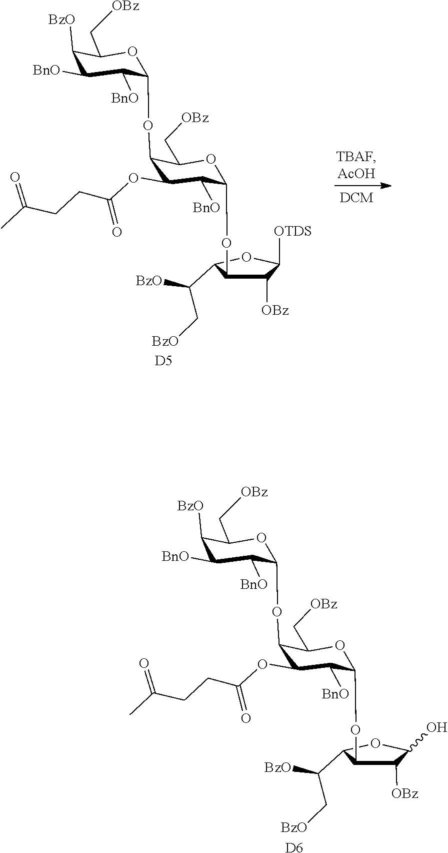

The TDS protected trisaccharide starting material D5 (28.4 g, 17.32 mmol) was dissolved in anhydrous DCM (139 mL). AcOH (20.5 mL, 358 mmol) was added. The solution was stirred for 5 min and TBAF (350 mL, 1 M in THF) was added. The reaction mixture was stirred at room temperature and monitored by TLC. After 16 h, the mixture was diluted with water (50 mL) and DCM (100 mL). The reaction mixture was quenched with sat. aq. sol. NaHCO₃ (150 mL). The organic layer was separated and washed with brine (150 mL). The organic layer was dried over Na₂SO₄, filtered and evaporated to give crude product. The crude was charged on isolute and purified using the automated purification system with Cy/EtOAc (0-100%) to give the product D6 as a white foam (25 g, 96%). HRMS calcd for C₈₆H₈₄NO₂₄+[M+NH₄]⁺ 1514.5378, found 1514.542.

### Synthesis of Trisaccharide Imidate Donor D7:

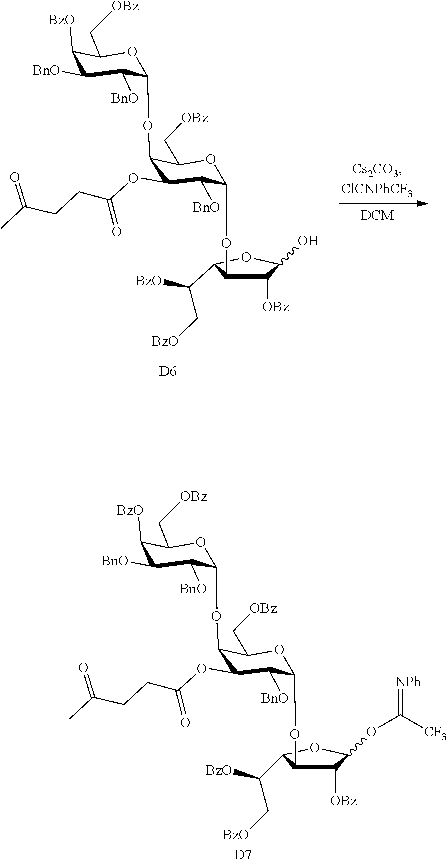

The trisaccharide hemiacetal starting material D6 (14.5 g, 9.68 mmol) was dissolved in anhydrous DCM (97 mL). Cs₂CO₃ (9.46 g, 29.0 mmol) and 2,2,2-trifluoro-N-phenylacetimidoyl chloride (4.02 g, 19.36 mmol) were added. The reaction mixture was stirred at room temperature and monitored by TLC. After 4.5 h, the reaction mixture was filtered through Celite. The solvent was evaporated to give the crude product. The crude was charged on isolute and purified using the automated purification system with Cy/EtOAc (0-100%, with 0.1% triethylamine) to give the product D7 as a white foam (13.5 g, 84%). HRMS calcd for C₉₄H₈₄F₃NNaO₂₄⁺ [M+Na]⁺ 1690.5228, found 1691.536.

### Synthesis of Undecasaccharide D8:

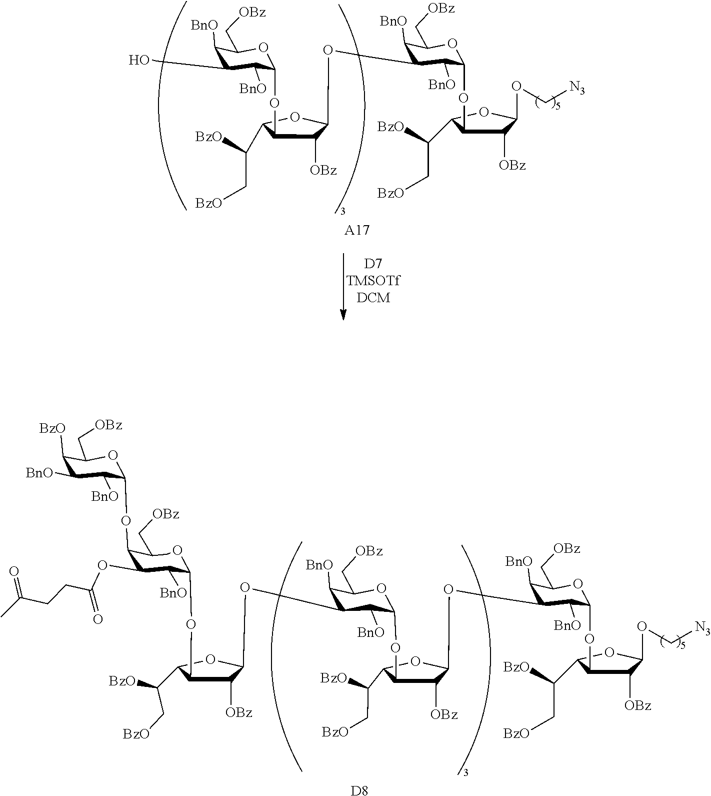

To a solution of the donor D7 (0.44 g, 0.26 mmol) and acceptor A17 ((see WO2019106201, page 248), 0.83 g, 0.22 mmol) in anhydrous DCM (9 mL) was added 4 Å MS and mixture stirred for 30 min. The reaction mixture was cooled to 0° C. TMSOTf (0.008 mL, 0.044 mmol) was added and the reaction mixture stirred for 30 min at the same temperature. Reaction was diluted with DCM (10 mL), filtered and quenched by addition of sat. aq. sol. NaHCO₃ (5 mL). The organic layer was separated, dried over Na₂SO₄ and filtered. The solvent was evaporated to give an oil residue. The crude reaction mixture was purified by automated purification system using Cy/EtOAc (0-100%) to give the product D8 (0.88 g, 78%). MALDI-TOF calcd for C₃₀₇H₂₈₁N₃NaO₈₀⁺ [M+Na]⁺ 5311.7904, found 5315.83.

### Synthesis of Undecasaccharide Acceptor D9:

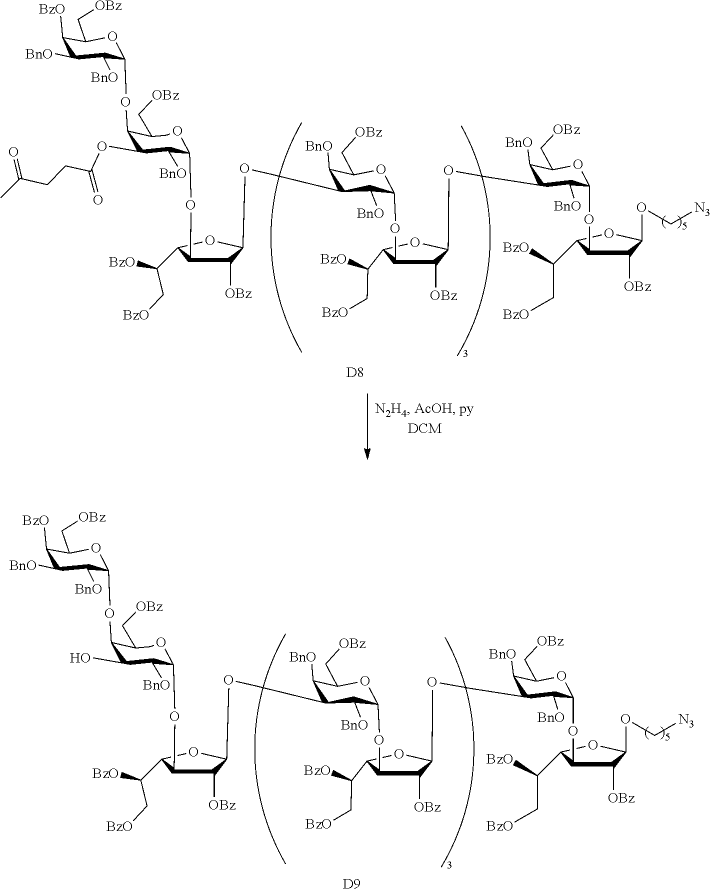

To a solution of the Lev protected undecasaccharide D8 (1.95 g, 0.375 mmol) in DCM (10 mL), a solution of hydrazine hydrate (0.12 mL, 3.75 mmol) dissolved in AcOH (0.8 mL) and py (1.2 mL) was added. The resulting reaction mixture was stirred at room temperature for 2 h. The reaction was quenched by the addition of acetone (1 mL) and the solvent removed under vacuum to obtain the crude product. The crude product was purified by automated flash chromatography using Cy/EtOAc (0-100%) as the eluent. Concentration of solvent from test tubes containing the product D9 (based on TLC) in vacuum resulted in a white foam (1.88 g, 98%). MALDI-TOF calcd for C₃₀₂H₂₇₅NNaO₇₈⁺ [M+Na-N₂]⁺ 5185.7475, found 5187.08.

### Synthesis 5-azido-pentyl 4,6-di-O-benzoyl-2,3-di-O-benzyl-α-D-galactopyranosyl-(1→4)-6-O-benzoyl-2-O-benzyl-3-O-levulinoyl-α-D-galactopyranosyl-(1→3)-2,5,6-tri-O-benzoyl-β-D-galactofuranosyl-(1→3)-4-[4,6-di-O-benzoyl-2,3-di-O-benzyl-α-D-galactopyranosyl-(1→)]-6-O-benzoyl-2-O-benzyl-α-D-galactopyranosyl-(1→3)-2,5,6-tri-O-benzoyl-β-D-galactofuranosyl-(1→3)-6-O-benzoyl-2,4-di-O-benzyl-α-D-galactopyranosyl-(1→3)-2,5,6-tri-O-benzoyl-β-D-galactofuranosyl-(1→3)-6-O-benzoyl-2,4-di-O-benzyl-α-D-galactopyranosyl-(1→3)-2,5,6-tri-O-benzoyl-β-D-galactofuranosyl-(1→3)-6-O-benzoyl-2,4-di-O-benzyl-α-D-galactopyranosyl-(1→3)-2,5,6-tri-O-benzoyl-β-D-galactofuranosyl-(1→3)]-6-O-benzoyl-2,4-di-O-benzyl-α-D-galactopyranosyl-(1→3)-2,5,6-tri-O-benzoyl-β-D-galactofuranoside D10

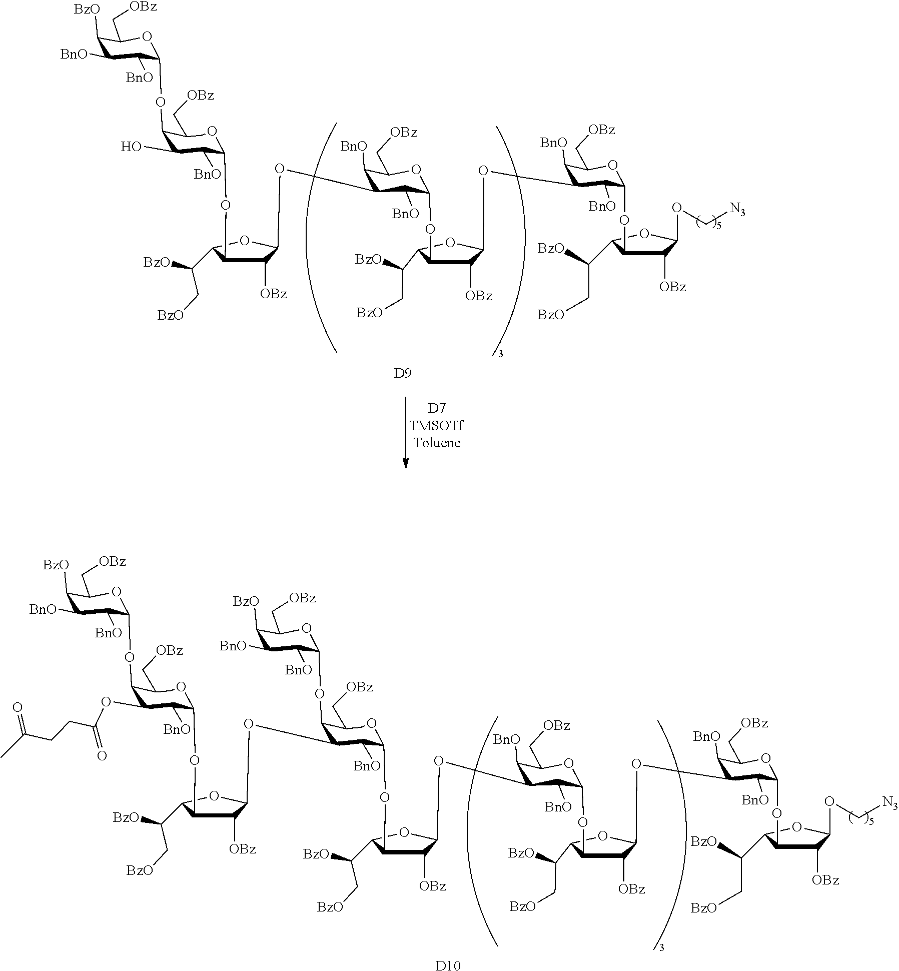

Acceptor D9 (1.45 g, 0.28 mmol) was coevaporated with toluene twice and dissolved in anhydrous toluene (8 mL), freshly activated 4 Å MS was added and stirred for 30 min. The reaction mixture was cooled to 0° C. and TMSOTf (5 μL, 0.028 mmol) was added dropwise. Donor D7 (0.56 g, 0.33 mmol) was coevaporated with toluene twice and dissolved in toluene (3 mL). Donor was added dropwise over 10 min to the reaction mixture. Toluene (1 mL) was added to wash the flask containing donor and the solution added to the reaction mixture dropwise. The reaction mixture was slowly warmed to 10° C. for 1.5 h. TLC showed complete consumption of the acceptor. Reaction was filtered, diluted with ethyl acetate (10 mL) and quenched by addition of sat. aq. sol. NaHCO₃ (10 mL). The organic layer was separated, dried over Na₂SO₄ and filtered. The solvent was evaporated to give an oil residue. The crude reaction mixture was purified by automated purification system using Cy/EtOAc (0-65%) to give the product D10 (1.4 g, 75%). MALDI-TOF calcd for C₃₈₈H₃₅₄N₃O₁₀₁⁺ [M+H]⁺ 6670.2651, found 6670.32. ¹H NMR (400 MHz, CDCl₃) δ 8.14-7.57 (m, 63H), 7.51-7.38 (m, 24H), 7.36-7.27 (m, 30H), 7.24-7.02 (m, 61H), 6.99-6.61 (m, 32H), 6.05 (s, 1H), 5.89-5.58 (m, 12H), 5.55-5.44 (m, 4H), 5.22-5.11 (m, 4H), 5.11-3.65 (m, 140H), 3.62-3.53 (m, 1H), 3.33-3.25 (m, 1H), 3.10 (t, J=6.9 Hz, 2H), 2.24-1.84 (m, 4H), 1.71 (s, 3H), 1.52-1.39 (m, 4H), 1.33-1.23 (m, 2H).

### Synthesis of Tetradecasaccharide D11:

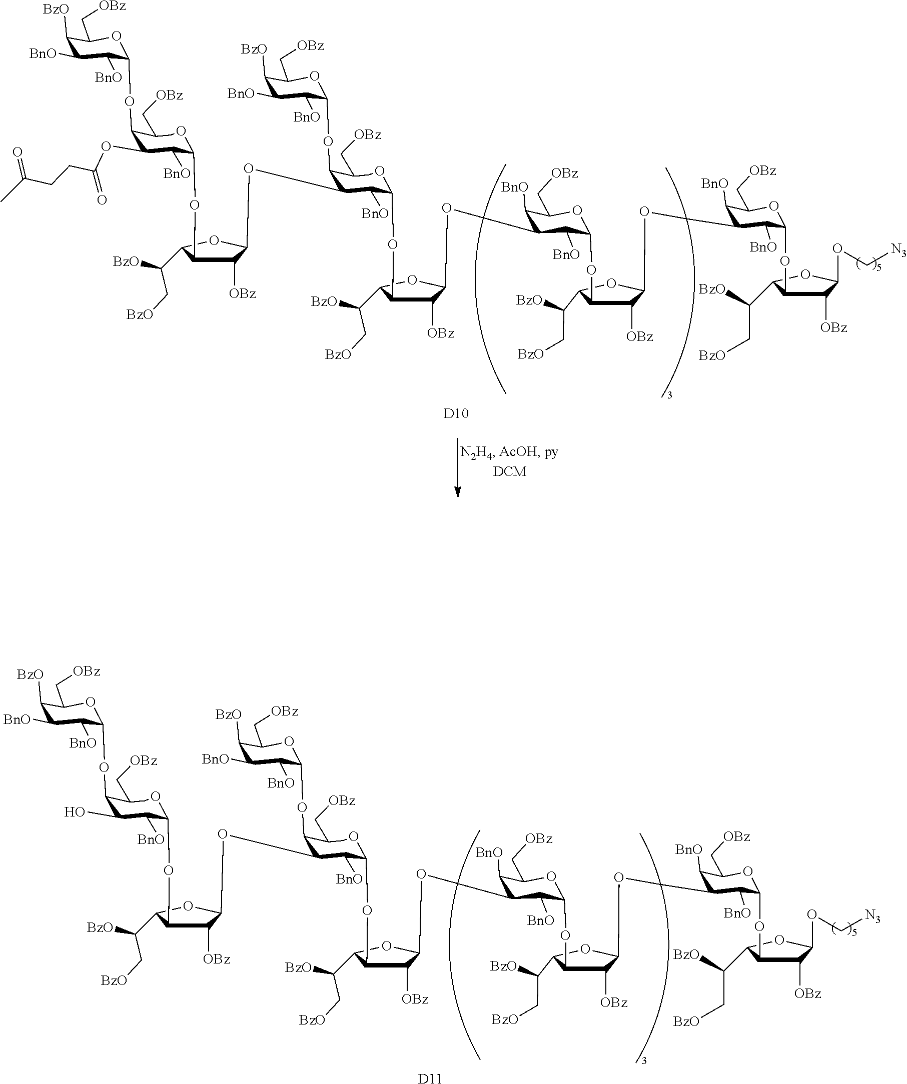

To a solution of the Lev protected tetradecasaccharide D10 (1.3 g, 0.195 mmol) in DCM (5 mL), a solution of hydrazine hydrate (0.06 mL, 1.95 mmol) dissolved in acetic acid (0.4 mL) and pyridine (0.6 mL) was added. The resulting reaction mixture was stirred at room temperature for 2 h. The reaction was quenched by the addition of acetone (1 mL) and the solvent removed under vacuum to obtain the crude product. The crude product was purified by automated flash chromatography using Cy/EtOAc (0-100%) as the eluent. Concentration of solvent from test tubes containing the product D11 (based on TLC) in vacuum resulted in a white foam (1.08 g, 84%). MALDI-TOF calcd for C₃₈₃H₃₄₇NNaO₉₉⁺ [M+Na-N₂]⁺ 6566.2041, found 6568.51.

### Synthesis of Partially Protected Tetradecasaccharide D12:

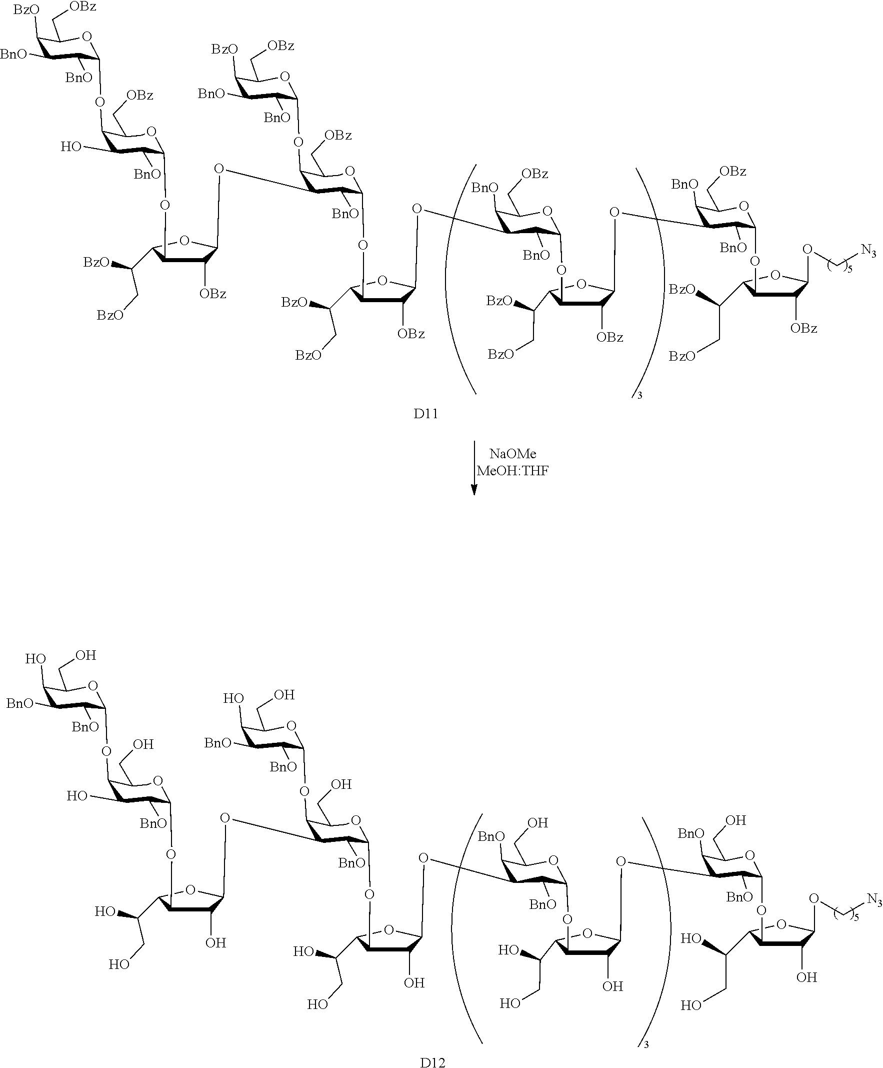

To a solution of the tetradecasaccharide D11 (275 mg, 0.042 mmol) in THF (5 mL) at rt, was added excess of 0.5 M NaOMe solution in methanol (2.93 mL, 1.464 mmol). The reaction mixture was warmed to 55° C. and stirred for 18 h. Then, the solvent was evaporated in vacuum to dryness. Water was added to the reaction mixture and neutralized with acetic acid. Water layer was extracted with EtOAc (3×10 mL). Combined organics were washed with sat. NaHCO₃ (2×10 mL), brine (10 mL), dried (Na₂SO₄), evaporated in vacuum to get crude product. SEC purification on LH-20 was done using 50% CHCl₃/MeOH as eluent. Fractions containing sugar stain active spots were collected and evaporated and dried in vacuum (125 mg). ¹H NMR and MALDI-TOF analysis showed that still few benzoyl groups were present in the molecule. The substrate (125 mg, 0.031 mmol) was taken in THF (5 mL) at rt and 0.5 M NaOMe solution in methanol (1.53 mL, 0.766 mmol) to the resulting solution. The reaction mixture was stirred at 60° C. for 18 h. The reaction solvent was evaporated in vacuum to dryness. Water was added to the residue and neutralized with acetic acid. The water layer was extracted with EtOAc (3×10 mL). Combined organics were washed with sat. NaHCO₃ (2×10 mL), brine (10 mL), dried (Na₂SO₄), evaporated in vacuum to get crude product. SEC purification on LH-20 was done using 30% CHCl₃/MeOH as eluent. Fractions containing sugar stain active spots were collected and evaporated and dried in vacuum to afford pale yellow colored fluffy solid D12 (90 mg, 59%). MALDI-TOF Calcd for C₁₈₇H₂₃₉N₄O₇₁⁺ [M+NH₄]⁺ 3676.5209, found 3677.00.

### Synthesis of 5-amino-pentyl α-D-galactopyranosyl-(1→4)-α-D-galactopyranosyl-(1→3)-β-D-galactofuranosyl-(1→3)-4-[α-D-galactopyranosyl-(1→)]-α-D-galactopyranosyl-(1→3)-β-D-galactofuranosyl-(1→3)-α-D-galactopyranosyl-(1→3)-β-D-galactofuranosyl-(1→3)-α-D-galactopyranosyl-(1→3)-β-D-galactofuranosyl-(1→3)-α-D-galactopyranosyl-(1→3)-β-D-galactofuranosyl-(1→3)-α-D-galactopyranosyl-(1→3)-β-D-galactofuranoside D13

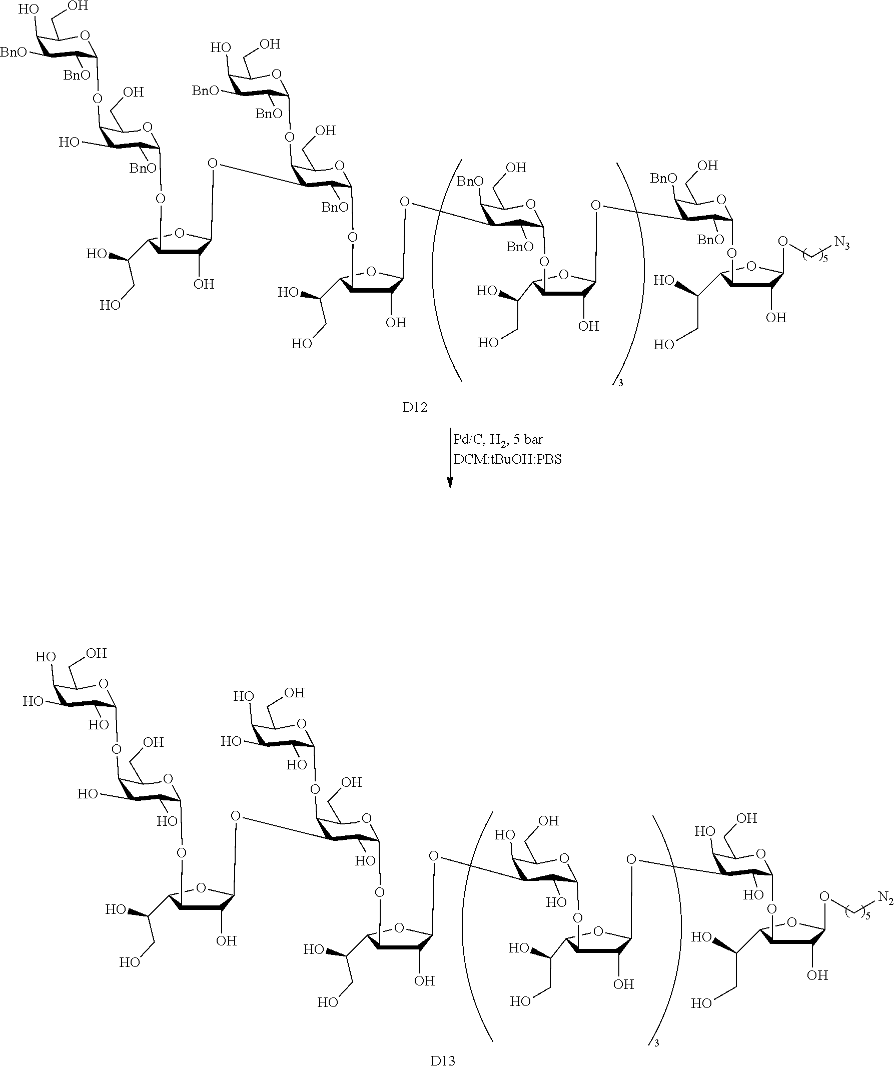

Debenzoylated tetradecasaccharide 012 (50 mg) was taken in a mixture of tBuOH:DCM:PBS (3:0.75:0.38) mL. Pd/C (100 mg) was added and hydrogenated under ~5 bar H₂ atmosphere for 20 h. Reaction mixture was filtered through the PTFE filter using 50% methanol in water (3×6 mL). The filtrate was concentrated under vacuum to get the crude as a white solid. The crude product was purified using Sep-Pak C18 (0.5 g) column using water-acetonitrile gradient as the eluent to get desired product as fluffy white solid after lyophilization. The product was further purified by SEC column on LH-20 resin using water as the eluent and fractions containing product D13 were combined, frozen and lyophilized to afford fluffy white solid (19 mg, 59%). ¹H NMR (400 MHz, D₂O) δ 5.20 (s, 5H), 5.11 (d, J=3.8 Hz, 1H), 5.09-5.05 (m, 6H), 5.03 (d, J=1.5 Hz, 1H), 4.95 (d, J=3.8 Hz, 1H), 4.43-4.37 (m, 4H), 4.35-3.53 (m, 82H), 3.03-2.96 (m, 2H), 1.74-1.60 (m, 4H), 1.50-1.38 (m, 2H). MALDI-TOF calcd for C₈₉H₁₅₃NNaO₇₁⁺ [M+Na]⁺ 2394.8285, found 2394.83.

### Synthesis of 5-azido-pentyl 2,3,5,6-tetra-O-benzoyl-β-D-galactofuranosyl-(1→3)-4-[4,6-di-O-benzoyl-2,3-di-O-benzyl-α-D-galactopyranosyl-(1→)]-6-O-benzoyl-2-O-benzyl-α-D-galactopyranosyl-(1→3)-2,5,6-tri-O-benzoyl-β-D-galactofuranosyl-(1→3)-4-[4,6-di-O-benzoyl-2,3-di-O-benzyl-α-D-galactopyranosyl-(1→)]-6-O-benzoyl-2-O-benzyl-α-D-galactopyranosyl-(1→3)-2,5,6-tri-O-benzoyl-β-D-galactofuranosyl-(1→3)-6-O-benzoyl-2,4-di-O-benzyl-α-D-galactopyranosyl-(1→3)-2,5,6-tri-O-benzoyl-β-D-galactofuranosyl-(1→3)-6-O-benzoyl-2,4-di-O-benzyl-α-D-galactopyranosyl-(1→3)-2,5,6-tri-O-benzoyl-β-D-galactofuranosyl-(1→3)-6-O-benzoyl-2,4-di-O-benzyl-α-D-galactopyranosyl-(1→3)-2,5,6-tri-O-benzoyl-β-D-galactofuranosyl-(1→3)]-6-O-benzoyl-2,4-di-O-benzyl-α-D-galactopyranosyl-(1→3)-2,5,6-tri-O-benzoyl-β-D-galactofuranoside D15

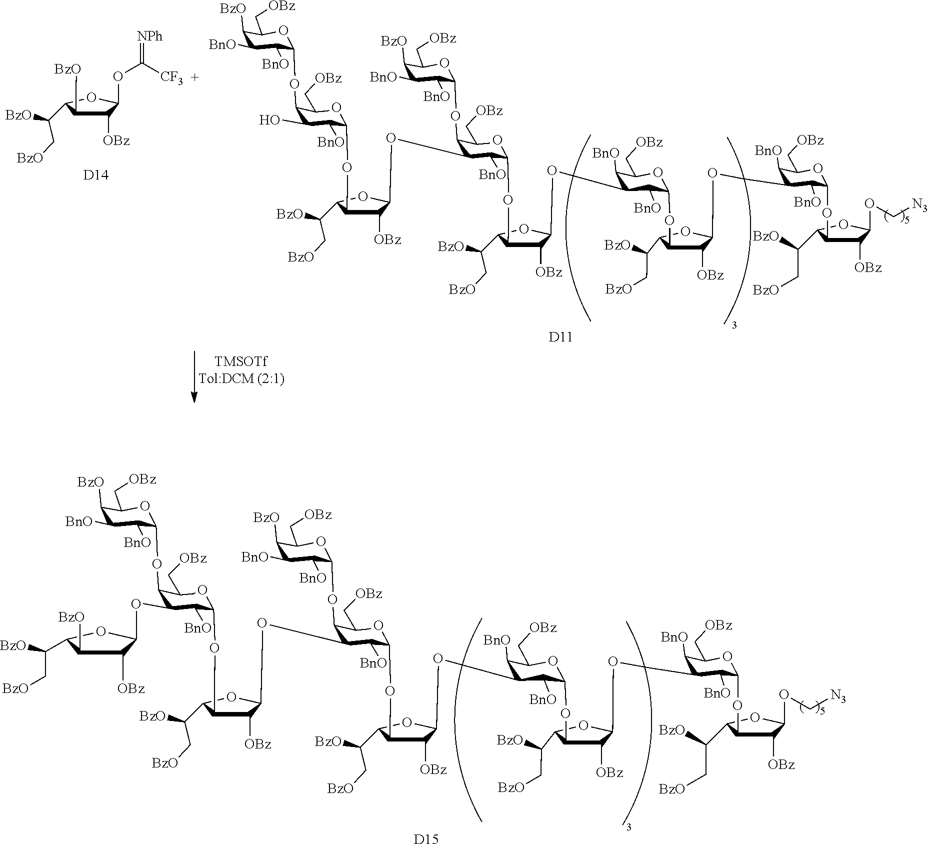

Acceptor D11 (0.25 g, 0.038 mmol) was coevaporated with toluene twice and dissolved in anhydrous toluene (3 mL), freshly activated 4 Å MS was added and stirred for 30 min. The reaction mixture was cooled to 0° C. and TMSOTf (1.34 μL, 0.038 mmol, 0.1 mL solution in toluene) was added dropwise. Donor D14 (see WO2019106201, page 198) (0.088 g, 0.114 mmol) was coevaporated with toluene twice and dissolved in DCM (1.5 mL). Donor was added dropwise over 10 min to the reaction mixture. The reaction mixture was slowly warmed to 10° C. for 1.5 h. TLC showed complete consumption of the acceptor. Reaction was filtered, diluted with ethyl acetate (10 mL) and quenched by addition of sat. aq. sol. NaHCO₃ (10 mL). The organic layer was separated, dried over Na₂SO₄ and filtered. The solvent was evaporated to give the crude product. The crude reaction mixture was purified by automated purification system using Cy/EtOAc (0-100%) to give the product D15 as a white foam (0.17 g, 62%). MALDI-TOF calcd for C₄₁₇H₃₇₄N₃O₁₀₈⁺ [M+H]⁺ 7150.3860, found 7150.75. ¹H NMR (400 MHz, CDCl₃) δ 8.16-7.27 (m, 128H), 7.26-6.71 (m, 102H), 6.04 (s, 1H), 5.91 (s, 1H), 5.87-5.73 (m, 8H), 5.71 (s, 1H), 5.66-5.57 (m, 2H), 5.57-5.41 (m, 5H), 5.17-5.10 (m, 4H), 5.10-4.91 (m, 13H), 4.89-4.78 (m, 2H), 4.75-4.17 (m, 68H), 4.11-3.94 (m, 20H), 3.88-3.74 (dd, J=23.9, 10.4 Hz, 8H), 3.67-3.53 (m, 1H), 3.39-3.23 (m, 1H), 3.13 (t, J=6.9 Hz, 2H), 1.58-1.39 (m, 4H), 1.28 (q, J=7.7, 7.2 Hz, 2H).

### Synthesis of Partially Protected Pentadecasaccharide D16:

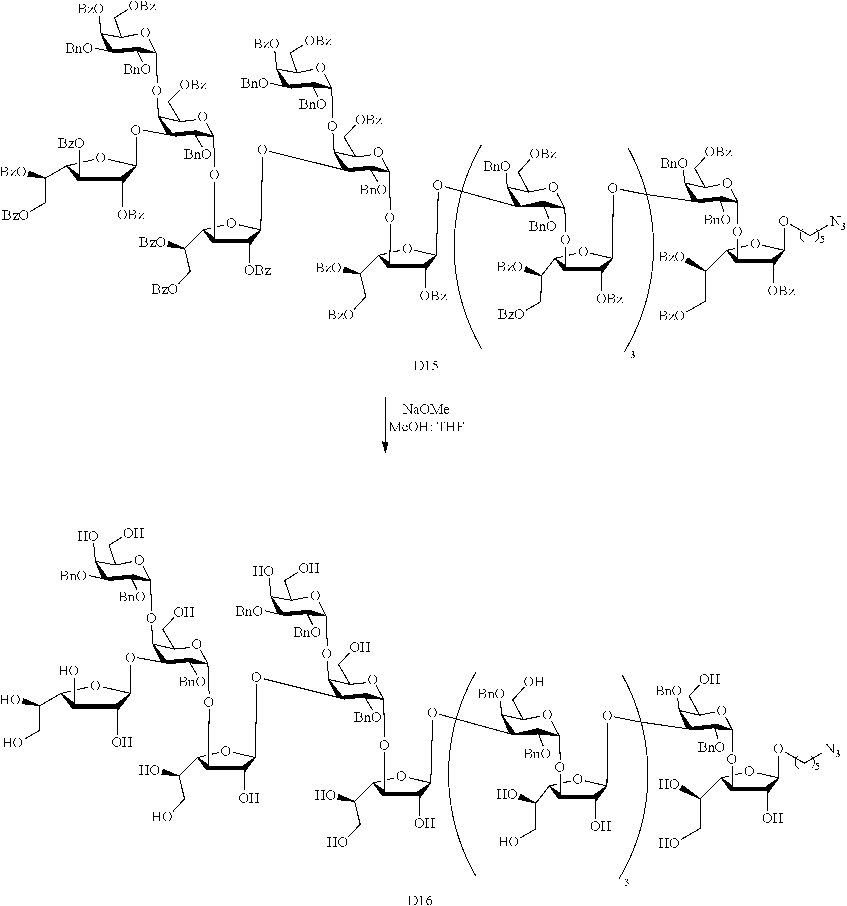

Sodium methoxide solution in MeOH 0.5 M (3 mL, 1.5 mmol) was added to a solution of the pentadecasaccharide D15 (160 mg, 0.022 mmol) in THF (3 mL). The reaction was stirred at 60° C. overnight. The reaction solvent was evaporated to give the crude product. The crude product was washed with H₂O (2×2 mL) and AcOH (0.1 mL in 1 mL H₂O). The solid was dissolved in MeOH and evaporated in rotavap. The residue was washed with cyclohexane and then purified by SEC using LH-20 CHCl₃:MeOH (1:2) to give the product D16 as a white solid (70 mg, 82%). MALDI-TOF calcd for C₁₉₃H₂₄₆N₃O₇₆⁺ [M+H]⁺ 3821.5471, found 3821.09.

### Synthesis of 5-amino-pentyl, β-D-galactofuranosyl-(1→3)-4-[α-D-galactopyranosyl-(1→)]-α-D-galactopyranosyl-(1→3)-β-D-galactofuranosyl-(1→3)-4-[α-D-galactopyranosyl-(1→)]-α-D-galactopyranosyl-(1→3)-β-D-galactofuranosyl-(1→3)-α-D-galactopyranosyl-(1→3)-β-D-galactofuranosyl-(1→3)-α-D-galactopyranosyl-(1→3)-β-D-galactofuranosyl-(1→3)-α-D-galactopyranosyl-(1→3)-β-D-galactofuranosyl-(1→3)-α-D-galactopyranosyl-(1→3)-β-D-galactofuranoside D17

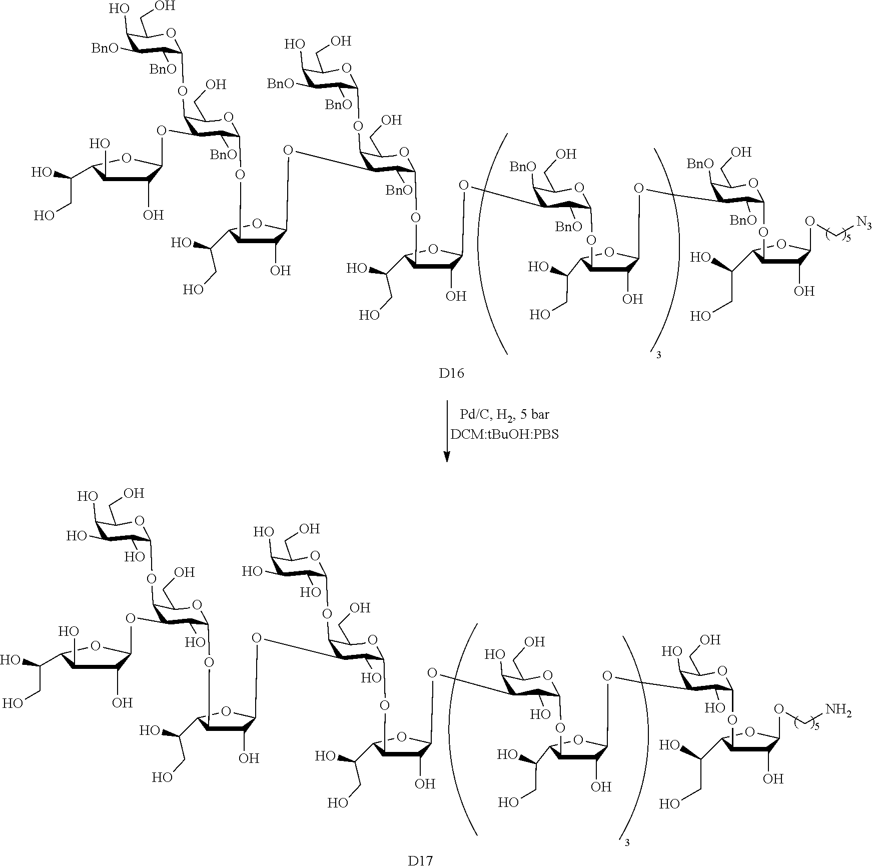

The debenzoylated pentadecasaccharide D16 (40 mg, 0.010 mmol) was dissolved in a mixture of DCM:tBuOH:H₂O (2:8:1, 3.5 mL). Pd/C (70 mg) was added and the reaction mixture was purged with hydrogen (5 times) and let stir under hydrogen pressure (5 bar) for 20 h. Then, the reaction mixture was filtered through PTFE filter using H₂O:ACN (1:1), the organic solvents evaporated in rotavapor and the crude material was lyophilized. The crude was purified by Sep-Pak C18 followed by SEC LH-20 using miliQ H₂O and lyophilized to give the product D17 as a white solid (12.9 mg, 49%). MALDI-TOF calcd for C₉₅H₁₆₃NNaO₇₆⁺ [M+Na]⁺ 2556.8813, found 2558.40. ¹H NMR (400 MHz, D₂O) δ 5.20 (s, 6H), 5.14-4.96 (m, 9H), 4.43-4.36 (m, 4H), 4.35-3.54 (m, 88H), 2.99 (t, J=7.5, 6.4 Hz, 2H), 1.75-1.56 (m, 4H), 1.50-1.36 (m, 2H).

### Synthesis of 5-azido-pentyl 4,6-di-O-benzoyl-2,3-di-O-benzyl-α-D-galactopyranosyl-(1→4)-6-O-benzoyl-2-O-benzyl-3-O-levulinoyl-α-D-galactopyranosyl-(1→3)-2,5,6-tri-O-benzoyl-β-D-galactofuranosyl-(1→3)-4-[4,6-di-O-benzoyl-2,3-di-O-benzyl-α-D-galactopyranosyl-(1→)]-6-O-benzoyl-2-O-benzyl-α-D-galactopyranosyl-(1→3)-2,5,6-tri-O-benzoyl-β-D-galactofuranosyl-(1→3)-4-[4,6-di-O-benzoyl-2,3-di-O-benzyl-α-D-galactopyranosyl-(1→)]-6-O-benzoyl-2-O-benzyl-α-D-galactopyranosyl-(1→3)-2,5,6-tri-O-benzoyl-β-D-galactofuranosyl-(1→3)-6-O-benzoyl-2,4-di-O-benzyl-α-D-galactopyranosyl-(1→3)-2,5,6-tri-O-benzoyl-β-D-galactofuranosyl-(1→3)-6-O-benzoyl-2,4-di-O-benzyl-α-D-galactopyranosyl-(1→3)-2,5,6-tri-O-benzoyl-β-D-galactofuranosyl-(1→3)-6-O-benzoyl-2,4-di-O-benzyl-α-D-galactopyranosyl-(1→3)-2,5,6-tri-O-benzoyl-β-D-galactofuranosyl-(1→3)]-6-O-benzoyl-2,4-di-O-benzyl-α-D-galactopyranosyl-(1→3)-2,5,6-tri-O-benzoyl-β-D-galactofuranoside D18

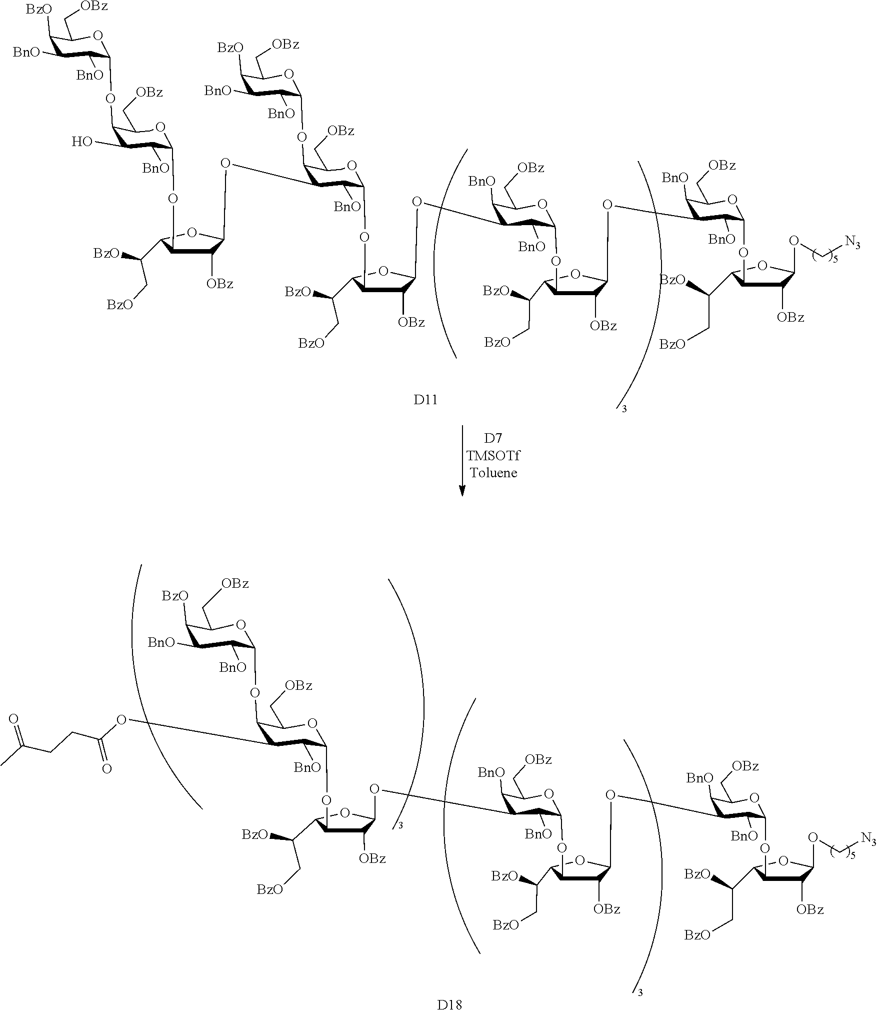

Acceptor D11 (0.55 g, 0.084 mmol) was coevaporated with toluene twice and dissolved in anhydrous toluene (4 mL), freshly activated 4 Å MS was added and stirred for 30 min. The reaction mixture was cooled to 0° C. and TMSOTf (3.02 μL, 0.017 mmol, 0.1 mL solution in toluene) was added dropwise. Donor D7 (0.28 g, 0.167 mmol) was coevaporated with toluene twice and dissolved in DCM (2 mL). Donor was added dropwise over 10 min to the reaction mixture. The reaction mixture was slowly warmed to 10° C. for 1.5 h. Reaction was filtered, diluted with ethyl acetate (10 mL) and quenched by addition of sat. aq. sol. NaHCO₃ (10 mL). The organic layer was separated, dried over Na₂SO₄ and filtered. The solvent was evaporated under vacuum to give the crude product. The crude reaction mixture was purified by automated purification system using Cy/EtOAc (0-100%) to give the product D18 as a white foam (0.46 g, 68%). MALDI-TOF calcd for C₄₆₉H₄₂₆N₃O₁₂₂⁺ [M+H]⁺ 8050.7217, found 8050.19. ¹H NMR (400 MHz, CDCl₃) δ 8.15-7.62 (m, 76H), 7.55-7.27 (m, 86H), 7.22-6.75 (m, 93H), 6.12 (s, 1H), 6.07-5.98 (d, J=12.2 Hz, 2H), 5.95-5.62 (m, 17H), 5.58-5.48 (d, J=11.3 Hz, 5H), 5.26-5.14 (m, 6H), 5.14-4.91 (m, 18H), 4.90-3.72 (m, 148H), 3.66-3.54 (m, 1H), 3.33 (d, J=9.7 Hz, 1H), 3.14 (t, J=6.9 Hz, 2H), 2.23-1.81 (m, 4H), 1.71 (s, 3H), 1.57-1.45 (m, 4H), 1.37-1.29 (m, 2H).

### Synthesis of Partially Protected Heptadecasaccharide D19:

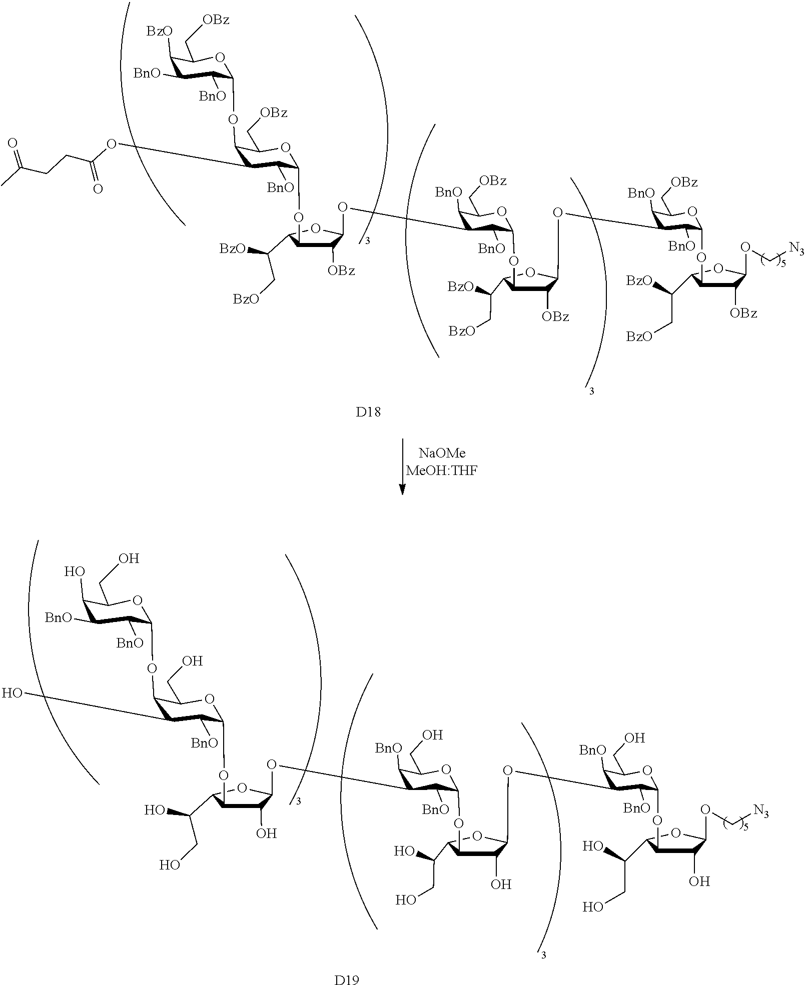

Sodium methoxide solution in MeOH 0.5 M (4 mL, 2 mmol) was added to a solution of the heptadecasaccharide D18 (160 mg, 0.022 mmol) in THF (3 mL). The reaction was stirred at 60° C. overnight. The reaction solvent was evaporated to give the crude product. The crude product was washed with H₂O (2×2 mL) and AcOH (0.1 mL in 1 mL H₂O). The solid was dissolved in MeOH and the solvent evaporated under vacuum to dryness. The residue was washed with cyclohexane and then purified by SEC using LH-20 CHCl₃:MeOH (1:2) to give the product D19 as a white solid (220 mg, 87%). MALDI-TOF calcd for C₂₂₆H₂₈₄N₃O₈₆⁺ [M+H]⁺ 4415.7936, found 4415.43.

### Synthesis of 5-amino-pentyl α-D-galactopyranosyl-(1-4)-α-D-galactopyranosyl-(1→3)-β-D-galactofuranosyl-(1→3)-4-[α-D-galactopyranosyl-(1→)]-α-D-galactopyranosyl-(1→3)-β-D-galactofuranosyl-(1→3)-4-[α-D-galactopyranosyl-(1→)]-α-D-galactopyranosyl-(1→3)-β-D-galactofuranosyl-(1→3)-α-D-galactopyranosyl-(1→3)-β-D-galactofuranosyl-(1→3)-α-D-galactopyranosyl-(1→3)-β-D-galactofuranosyl-(1→3)-α-D-galactopyranosyl-(1→3)-β-D-galactofuranosyl-(1→3)-α-D-galactopyranosyl-(1→3)-β-D-galactofuranoside D20

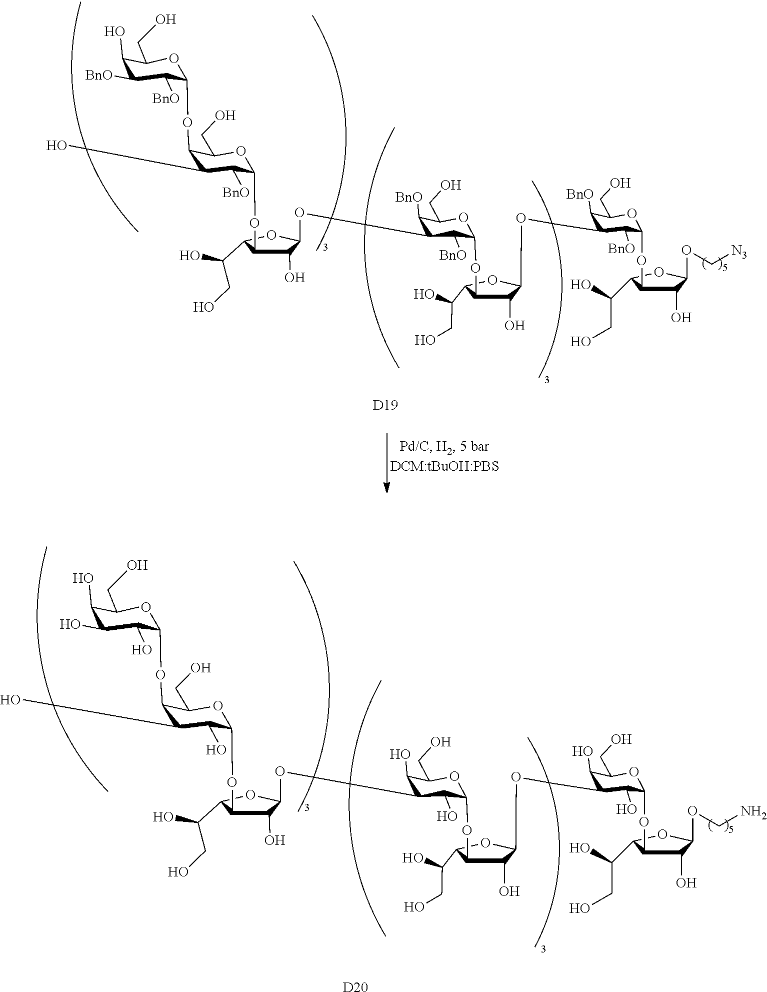

The debenzoylated heptadecasaccharide D19 (37 mg, 0.084 mmol) was dissolved in a mixture of DCM:tBuOH:H₂O (2:8:1, 2.75 mL). Pd/C (40 mg) was added and the reaction mixture was purged with hydrogen (5 times) and let stir under hydrogen pressure (5 bar) for 20 h. Then, the reaction mixture was filtered through PTFE filter using H₂O:ACN (1:1), the solvent was evaporated in rotavapor and the crude material was lyophilized. The crude was purified by Sep-Pak C18 column, followed by SEC LH-20 using miliQ H₂O and lyophilized to give the product D20 as a white solid (5.3 mg, 22%). MALDI-TOF calcd for C₁₀₇H₁₈₃NNaO₈₆⁺ [M+Na]⁺ 2880.9869, found 2882.40. ¹H NMR (400 MHz, D₂O) δ 5.25-5.17 (m, 6H), 5.13-4.98 (m, 10H), 4.94 (d, J=3.5 Hz, 1H), 4.41 (s, 4H), 4.33-4.03 (m, 40H), 3.95-3.80 (m, 32H), 3.76-3.56 (m, 28H), 2.98 (t, J=7.2 Hz, 2H), 1.75-1.58 (m, 4H), 1.51-1.36 (m, 2H).

### Synthesis of the Conjugates:

### Synthesis of the NHS Ester of Compound D13 (D13-Adipate-NHS)

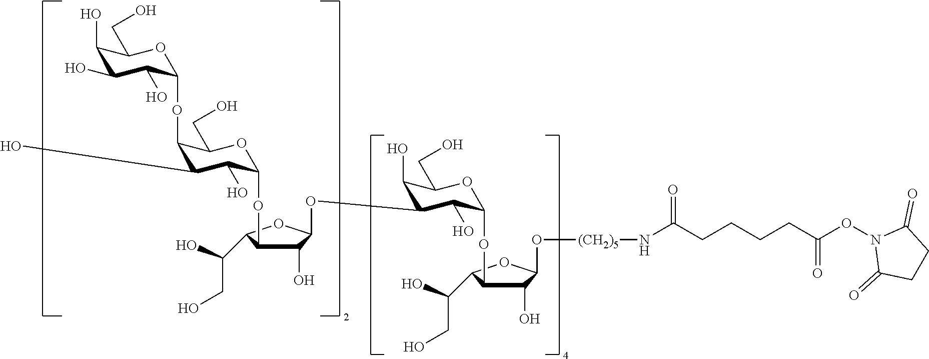

Compound D13 (4 mg, 1.686 μmol) was dissolved in DMSO-H₂O (100 μL-10 μL) at rt in a 15 mL falcon tube. Triethylamine (8 μL, 0.059 mmol) was added to it. Adipate-NHS ester (bis(2,5-dioxopyrrolidin-1-yl) adipate) (11.5 mg, 0.034 mmol) in DMSO (100 μL) was added and stirred for 2 h at rt. D13-adipate-NHS ester was precipitated by adding EtOAc (5 mL) and centrifuged, washed the precipitate with EtOAc (3 mL×2), dried in vacuum to get white solid (4 mg, 91%) and taken for the next step.

Synthesis of the D13 Conjugate with CRM₁₉₇ (D13-Adipate-CRM₁₉₇ or D13-CRM₁₉₇*)

Compound D13-adipate-NHS ester was dissolved in (4 mg, 1.54 μmol) in 0.1 M NaPi buffer (pH 7.0, 150 μL) in a 15 mL falcon tube. Freshly washed CRM₁₉₇ (obtained from EirGenix, Inc., Taiwan, expression system E. coli) (2 mg, 0.034 μmol) in 0.1 M NaPi buffer (pH 7.0, 150 μL) in a vial was added to it dropwise. The vial was rinsed with 0.1 M NaPi buffer (pH 7.0, 50 μL) and transferred to the reaction mixture in falcon tube and stirred at rt for 20 h. Obtained D13-adipate-CRM₁₉₇ solution was transferred to the Amicon Ultra vial (10 kDa, MWCO), centrifuged for 5 minutes at 2-8° C. temperature. Added 300 μL of 0.1 M NaPi to the reaction falcon tube, rinsed and transferred to the filter and centrifuged again. Additional washings were done using TBS buffer (pH 7.4) solution for five more times. After the final wash the conjugate was sterile-filtered and stored in TBS (1.0 mL) (pH 7.4) at 2-8° C. The loading found was 12.3 using MALDI-TOF MS. The conjugate was analysed using, SDS-PAGE, BCA, SEC-HPLC and for endotoxin.

Synthesis of the Conjugate with BSA (D13-Adipate-BSA or D13-BSA*)

Compound D13-adipate-NHS was dissolved in (1.7 mg, 0.654 μmol) in 0.1 M NaPi buffer (pH 7.0, 150 μL) in a 15 mL falcon tube. Freshly washed BSA (obtained from Sigma Aldrich, heat shock fraction, pH 7, ≥98%; product no. A7906) (3 mg, 0.045 μmol) in 0.1 M NaPi buffer (pH 7.0, 150 μL) in a vial was added to it dropwise. The vial was rinsed with 0.1 M NaPi buffer (pH 7.0, 50 μL) and transferred to the reaction mixture in falcon tube and stirred at rt for 20 h. Obtained D13-adipate-BSA solution was transferred to the Amicon Ultra vial (10 kDa, MWCO), centrifuged for 5 minutes at 2-8° C. temperature. Added 300 μL of 0.1 M NaPi to the reaction falcon tube, rinsed and transferred to the filter and centrifuged again. Additional washings were done using 1×PBS solution for five more times. After the final wash the conjugate was sterile-filtered and stored in PBS (0.5 mL) (pH 7.4) at 2-8° C. The loading found was 6.96 using MALDI-TOF MS. The conjugate was analysed using SDS-PAGE and SEC-HPLC.

### Synthesis of the NHS Ester of Compound D17 (D17-Adipate-NHS)

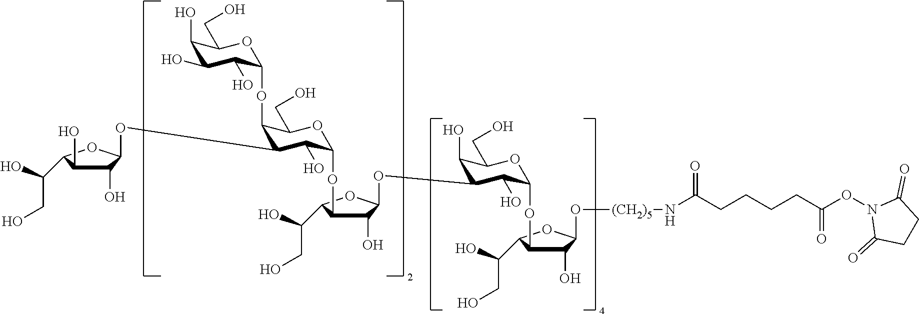

NHS ester of compound D17 was synthesized using similar procedure explained above for D13-adipate-NHS and dried in vacuum to get white solid (7 mg, 92%) and taken for the next step.

Synthesis of the D17 Conjugate with CRM₁₉₇ (D17-adipate-CRM₁₉₇ or D17-CRM₁₉₇*)

D17 conjugate with CRM₁₉₇ (3 mg) was synthesized using D17-adipate-NHS and CRM₁₉₇ using similar procedure explained above for D13-adipate-CRM₁₉₇ and the loading found was 9.69 using MALDI-TOF MS.

Synthesis of the Conjugate with BSA (D17-Adipate-BSA or D17-BSA*)

D17 conjugate with BSA (1 mg) was synthesized using D17-adipate-NHS and BSA using similar procedure explained above for D13-adipate-BSA and the loading found was 12.78 using MALDI-TOF MS.

### Synthesis of the NHS Ester of Compound D20 (D20-Adipate-NHS)

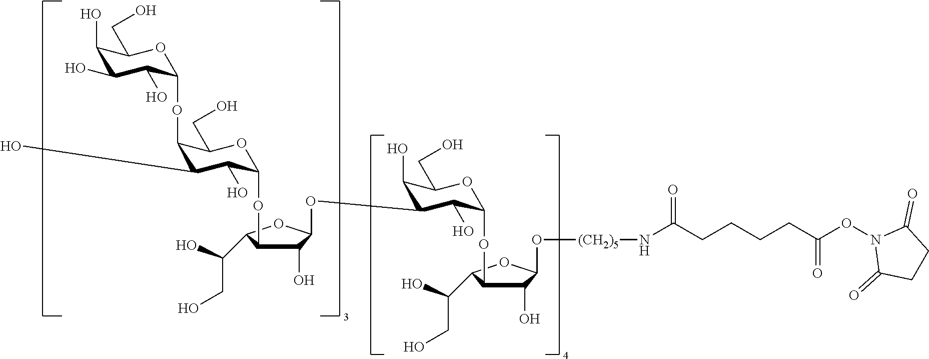

NHS ester of compound D20 was synthesized using similar procedure explained above for D13-adipate-NHS and dried in vacuum to get white solid (7 mg, 93%) and taken for the next step.

Synthesis of the D20 Conjugate with CRM₁₉₇ (D20-Adipate-CRM₁₉₇ or D20-CRM₁₉₇*)

D20 conjugate with CRM₁₉₇ (3 mg) was synthesized using D20-adipate-NHS and CRM₁₉₇ using similar procedure explained above for D13-adipate-CRM₁₉₇ and the loading found was 7.45 using MALDI-TOF MS.

Synthesis of the Conjugate with BSA (D20-Adipate-BSA or D20-BSA*)

D17 conjugate with BSA (1 mg) was synthesized using D17-adipate-NHS and BSA using similar procedure explained above for D13-adipate-BSA and the loading found was 9.41 using MALDI-TOF MS.

### II. Biology

### Materials:

ELISA plates (high-binding, EIA/RIA Plate, 96 well, flat bottom with low evaporation lid, company: Costar® 3361)
Detection antibody: Goat anti rabbit IgG peroxidase conjugate (Sigma, #A4914)
Blocking solution: Commercial blocking reagent (Roche, cat.no. 11112589001)
Antibody diluent: PBS+1% BSA (w/v)
Wash Buffer: PBS+0.1% Tween 20 (PBS-T)
Developing solution: 1 Step™ Ultra TMB-ELISA developer. (ThermoScientific, Cat #: 34028)
Stop solution—2M sulphuric acid (H₂SO₄)
Plate reader: FLUOstar Omega (BMG LABTECH)
Software: GraphPad Prism version 7 or higher for data plotting and analysis
Alum: Aluminium Hydroxide Adjuvant (Rehydragel® HPA), Chemtrade
QuantiPro™ BCA Assay Kit (SIGMA) Product: QPBCA-1KT; Lot #: SLBR7451V; Pcode: 1002296464
Mini-PROTEAN® TGX™ Gels—10%, 10 well (30 μL/well) Control Nr:64175708
GelCode™ Blue Safe Protein Stain; ThermoScientific; Ref: 1860957; Lot #: TA260266

### Methods:

### Bacterial Strains and LPS.

Klebsiella pneumoniae strains differing in their LPS (O-antigen) were used to isolate and purify the corresponding LPS. The purified LPS were used as coating antigen in Enzyme Linked Immunosorbent Assay (ELISA). LPS was isolated using a commercial LPS extraction kit (JH Science) according to the manufacturer's protocol.

TABLE 1
Klebsiella pneumoniae strains used for LPS isolation.

| # | LPS/O-antigen |
|---|---|
| 1 NCTC 9148 | O2a |
| 2 PCM27 | Galactan-III (O2afg) |

Formulation of vaccine candidates for immunization. All formulations were prepared under sterile conditions. Drug substance (DS) and buffer (10 mM TRIS-HCl, pH 7.4) were mixed in the appropriate pre-calculated dilution factor (see below) for the required glycan dose leaving out the required volume of aluminum hydroxide adjuvant (0.25 mg/mL). The DS-buffer mixture was gently mixed and aluminum hydroxide adjuvant (“aluminum”) stock was added for a final aluminum concentration of 0.250 mg/mL of aluminum. The mixture was immediately mixed by gentle pipetting and then mixed on a horizontal shaker at 250 rpm for 2 h at RT. Aliquots were stored in type 1 glass vials at 4° C. until further use.

The vaccines described above are prepared to contain the intended glycan dose (e.g., 2 μg glycan per injection) as follows. The average loading factor of the glycan antigen (in moles of antigen per mol carrier protein) is determined via MALDI-TOF MS by subtracting the determined molecular weight (m/z=1) of CRM₁₉₇ from the determined molecular weight of the DS (m/z=1), then this mass difference is divided by the theoretical molecular weight of the glycan antigen including the linker (here: alkyl) and spacer (here: adipoyl) moieties. The resulting loading factor is multiplied by the theoretical molecular weight of the glycan antigen excluding the linker and spacer moieties, providing the total mass of glycan attached on average per DS molecule. This total mass of glycan is divided by the determined molecular weight of the CRM₁₉₇ protein to yield the glycan-to-protein mass ratio of the DS. This ratio is multiplied by the determined protein concentration of the DS, as determined by the BCA Assay Kit (Sigma) according to the manufacturer's protocol, to yield the glycan concentration of the DS. To obtain the dilution factor necessary to dilute the DS to obtain the intended glycan dose per immunization, the glycan concentration of the DS is divided by the required glycan concentration (e.g., 4 μg/mL glycan concentration for a 2 μg glycan dose for rabbits with an injection volume of 500 μL). The DS is then diluted with this dilution factor relative to the final volume of the vaccine preparation.

Immunizations: Female Zika rabbits were immunized via the intramuscular (i.m.) route with an injection volume of 500 μL per dose. Animals were kept under specific pathogen-free conditions and were provided with water and food ad libitum.

ELISA: Coating of plates with antigen: Isolated LPS were used for coating. LPS was dissolved in isopropanol to a concentration of 10 μg/mL and 100 μL was used for coating so that each well was coated with 1 μg of LPS. LPS solutions were subjected to overnight evaporation at RT inside the biosafety cabinet. Blocking: The plates were blocked using 100 μL of commercial blocking solution and incubated for 1 h at RT. After blocking, the plates were washed 3× with PBS with 0.1% (v/v) Tween-20 (PBS-T). Incubation with diluted sera: Pooled or individual sera from different timepoints were diluted to their respective dilutions using 1% BSA (w/v) in PBS. 50-100 μL of the diluted sera were added in duplicates to the ELISA wells and incubated for 1 h at RT. 100 μL/well of 1% BSA (w/v) in PBS served as blank. After incubation with sera, the plates were washed 3× with PBS-T. Incubation with detection antibody: Anti-rabbit IgG HRP conjugate was diluted 1:10,000 in 1% BSA (w/v) in PBS and 100 μL/well were added and incubated for 30 minutes at RT. After the incubation with detection antibody, the plates were washed 3× with PBS-T. Substrate addition: To each well, 100 μL of TMB substrate were added and incubated for approx. 15 min. The reaction was stopped by adding 50 μL/well of 2M H₂SO₄. Absorption was measured at 450 nm using a plate reader. The absorption values were analyzed with the GraphPad Prism software.

Challenge experiments: Female CD-1 mice received pooled post-immune antisera (30-250 μL) from rabbits via the intraperitoneal route 24 hours and 1 hour before bacterial challenge. Prior to bacterial challenge, mice were pre-treated with 20 mg per animal of galactosamine. Mice were either infected with 2×10⁷ colony-forming units (CFU) of K. pneumoniae strain NCTC 9163 (O2a) or 1×10⁸ CFU of strain ST258 (Gal-III) via the intraperitoneal route. Mice were observed for 24 hours for clinical scores and sacrificed at humane endpoint.

(CRM₁₉₇)
SEQ ID NO: 1

| | |
|---|---|
| GADDVVDSSK SFVMENFSSY HGTKPGYVDS | 30 |
| IQKGIQKPKS GTQGNYDDDW KEFYSTDNKY | 60 |
| DAAGYSVDNE NPLSGKAGGV VKVTYPGLTK | 90 |
| VLALKVDNAE TIKKELGLSL TEPLMEQVGT | 120 |
| EEFIKRFGDG ASRVVLSLPF AEGSSSVEYI | 150 |
| NNWEQAKALS VELEINFETR GKRGQDAMYE | 180 |
| YMAQACAGNR VRRSVGSSLS CINLDWDVIR | 210 |
| DKTKTKIESL KEHGPIKNKM SESPNKTVSE | 240 |
| EKAKQYLEEF HQTALEHPEL SELKTVTGTN | 270 |
| PVFAGANYAA WAVNVAQVID SETADNLEKT | 300 |
| TAALSILPGI GSVMGIADGA VHHNTEEIVA | 330 |
| QSIALSSLMV AQAIPLVGEL VDIGFAAYNF | 360 |
| VESIINLFQV VHNSYNRPAY SPGHKTQPFL | 390 |
| HDGYAVSWNT VEDSIIRTGF QGESGHDIKI | 420 |
| TAENTPLPIA GVLLPTIPGK LDVNKSKTHI | 450 |
| SVNGRKIRMR CRAIDGDVTF CRPKSPVYVG | 480 |
| NGVHANLHVA FHRSSSEKIH SNEISSDSIG | 510 |
| VLGYQKTVDH TKVNSKLSLF FEIKS | 535 |

(diphtheria toxin (Uniprot ID: P00587))
SEQ ID NO: 2

| | |
|---|---|
| GADDVVDSSK SFVMENFSSY HGTKPGYVDS | 30 |
| IQKGIQKPKS GTQGNYDDDW KGFYSTDNKY | 60 |
| DAAGYSVDNE NPLSGKAGGV VKVTYPGLTK | 90 |
| VLALKVDNAE TIKKELGLSL TEPLMEQVGT | 120 |
| EEFIKRFGDG ASRVVLSLPF AEGSSSVEYI | 150 |
| NNWEQAKALS VELEINFETR GKRGODAMYE | 180 |
| YMAQACAGNR VRRSVGSSLS CINLDWDVIR | 210 |
| DKTKTKIESL KEHGPIKNKM SESPNKTVSE | 240 |
| EKAKQYLEEF HQTALEHPEL SELKTVTGTN | 270 |
| PVFAGANYAA WAVNVAQVID SETADNLEKT | 300 |
| TAALSILPGI GSVMGIADGA VHHNTEEIVA | 330 |
| QSIALSSLMV AQAIPLVGEL VDIGFAAYNF | 360 |
| VESIINLFQV VHNSYNRPAY SPGHKTQPFL | 390 |
| HDGYAVSWNT VEDSIIRTGF QGESGHDIKI | 420 |
| TAENTPLPIA GVLLPTIPGK LDVNKSKTHI | 450 |
| SVNGRKIRMR CRAIDGDVTF CRPKSPVYVG | 480 |
| NGVHANLHVA FHRSSSEKIH SNEISSDSIG | 510 |
| VLGYQKTVDH TKVNSKLSLF FEIKS | 535 |

(tetanus toxin (Uniprot ID: P04958))
SEQ ID NO: 3

| | |
|---|---|
| PITINNFRYS DPVNNDTIIM MEPPYCKGLD | 30 |
| IYYKAFKITD RIWIVPERYE FGTKPEDFNP | 60 |
| PSSLIEGASE YYDPNYLRTD SDKDRFLQTM | 90 |
| VKLFNRIKNN VAGEALLDKI INAIPYLGNS | 120 |
| YSLLDKFDTN SNSVSFNLLE QDPSGATTKS | 150 |
| AMLTNLIIFG PGPVLNKNEV RGIVLRVDNK | 180 |
| NYFPCRDGFG SIMQMAFCPE YVPTFDNVIE | 210 |
| NITSLTIGKS KYFQDPALLL MHELIHVLHG | 240 |
| LYGMQVSSHE IIPSKQEIYM QHTYPISAEE | 270 |
| LFTFGGQDAN LISIDIKNDL YEKTLNDYKA | 300 |
| IANKLSQVTS CNDPNIDIDS YKQIYQQKYQ | 330 |
| FDKDSNGQYI VNEDKFQILY NSIMYGFTEI | 360 |
| ELGKKFNIKT RLSYFSMNHD PVKIPNLLDD | 390 |
| TIYNDTEGFN IESKDLKSEY KGQNMRVNTN | 420 |
| AFRNVDGSGL VSKLIGLCKK IIPPTNIREN | 450 |
| LYNRTASLTD LGGELCIKIK NEDLTFIAEK | 480 |
| NSFSEEPFQD EIVSYNTKNK PLNFNYSLDK | 510 |
| IIVDYNLQSK ITLPNDRTTP VTKGIPYAPE | 540 |
| YKSNAASTIE IHNIDDNTIY QYLYAQKSPT | 570 |
| TLQRITMTNS VDDALINSTK IYSYFPSVIS | 600 |
| KVNQGAQGIL FLOWVRDIID DFTNESSQKT | 660 |
| TIDKISDVST IVPYIGPALN IVKQGYEGNE | 690 |
| IGALETTGVV LLLEYIPEIT LPVIAALSIA | 720 |
| ESSTQKEKII KTIDNFLEKR YEKWIEVYKL | 750 |
| VKAKWLGTVN TQFQKRSYQM YRSLEYQVDA | 780 |
| IKKIIDYEYK IYSGPDKEQI ADEINNLKNK | 810 |
| LEEKANKAMI NINIFMRESS RSFLVNQMIN | 840 |
| EAKKQLLEFD TOSKNILMQY IKANSKFIGI | 870 |
| TELKKLESKI NKVFSTPIPF SYSKNLDCWV | 900 |
| DNEEDIDVIL KKSTILNLDI NNDIISDISG | 930 |
| FNSSVITYPD AQLVPGINGK AIHLVNNESS | 960 |
| EVIVHKAMDI EYNDMFNNFT VSFWLRVPKV | 990 |
| SASHLEQYGT NEYSIISSMK KHSLSIGSGW | 1020 |
| SVSLKGNNLI WTLKDSAGEV RQITFRDLPD | 1050 |
| KFNAYLANKW VFITITNDRL SSANLYINGV | 1080 |
| LMGSAEITGL GAIREDNNIT LKLDRCNNNN | 1100 |
| QYVSIDKFRI FCKALNPKEI EKLYTSYLSI | 1130 |
| TFLRDFWGNP LRYDTEYYLI PVASSSKDVQ | 1160 |
| LKNITDYMYL TNAPSYTNGK LNIYYRRLYN | 1190 |
| GLKFIIKRYT PNNEIDSFVK SGDFIKLYVS | 1200 |
| YNNNEHIVGY PKDGNAFNNL DRILRVGYNA | 1230 |
| PGIPLYKKME AVKLRDLKTY SVQLKLYDDK | 1260 |
| NASLGLVGTH NGQIGNDPNR DILIASNWYF | 1290 |
| NHLKDKILGC DWYFVPTDEG WIND | 1314 |

(cholera toxin B subunit (Uniprot ID: P01556))
SEQ ID NO: 4

| | |
|---|---|
| TPQNITDLCA EYHNTQIYTL NDKIFSYTES | 30 |
| LAGKREMAII TFKNGAIFQV EVPGSQHIDS | 60 |
| QKKAIERMKD TLRIAYLTEA KVEKLCVWNN | 90 |
| KTPHAIAAIS MAN | 103 |

(Neisseria meningitidis outer membrane protein
(OMP) (Uniprot ID: Q51229))
SEQ ID NO: 5

| | |
|---|---|
| MKKTVFTCAM IALTGTAAAA QELQTANEFT | 30 |
| VHTDLSSISS TRAFLKEKHK AAKHISVRAD | 60 |
| IPFDANQGIR LEAGFGRSKK NIINLETDEN | 90 |
| KLGKTKNVKL PTGVPENRID LYTGYTYTQT | 120 |
| LSDSLNFRVG AGLGFESSKD SIKTTKHTLH | 150 |
| SSRQSWLAKV HADLLSQLGN GWYINPWSEV | 180 |
| KFDLNSRYKL NTGVTNLKKD INQKINGWGF | 210 |
| GLGANIGKKL GESASIEAGP FYKORTYKES | 240 |
| GEFSVTTKSG DVSLTIPKTS IREYGLRVGI | 270 |
| KF | 272 |

(capsid protein of bacteriophage Qβ (Uniprot ID:
P03615))
SEQ ID NO: 6

| | |
|---|---|
| AKLETVTLGN IGKDGKQTLV LNPRGVNPTN | 30 |
| GVASLSQAGA VPALEKRVTV SVSQPSRNRK | 60 |
| NYKVQVKIQN PTACTANGSC DPSVTRQAYA | 90 |
| DVTFSFTQYS TDEERAFVRT ELAALLASPL | 120 |
| LIDAIDOLNP AY | 132 |

1. An immunogenic compound comprising at least one oligosaccharide hybrid antigen having the structure of formula (I) 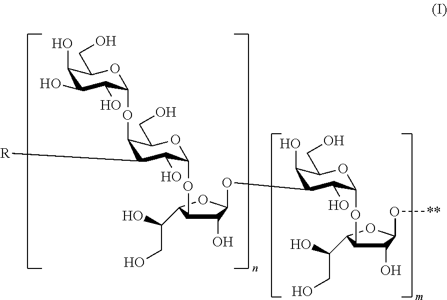
wherein
R is OH, or 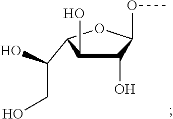
m is 3, 4, 5, 6, 7 or 8; and
n is 1, 2, 3, 4, 5 or 6;
or a pharmaceutically acceptable salt thereof.

2. The immunogenic compound according to claim 1, or a pharmaceutically acceptable salt thereof, wherein
m is 4, n is 2 and R is OH;
m is 4, n is 2 and R is 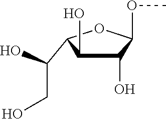
or;
m is 4, n is 3 and R is OH.

3. The immunogenic compound according to claim 1, or a pharmaceutically acceptable salt thereof, wherein m is 4, n is 2 and R is OH.

4. An immunogenic compound of formula (II) 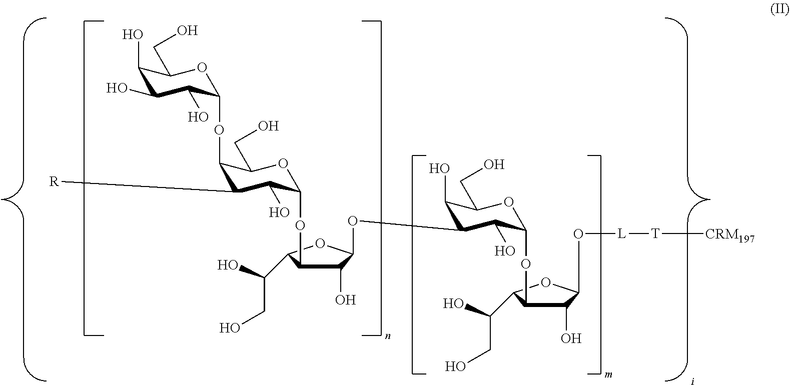
wherein
R is OH, or 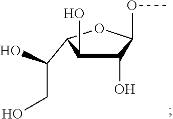
m is 3, 4, 5, 6, 7 or 8;
n is 1, 2, 3, 4, 5 or 6;
i is from 1 to 28; and
-L-T- represents a linker L and a spacer T which together form a bridge having a backbone with a length of 5 to 25 atoms covalently linked together that forms the shortest distance between the oxygen at C1 of the reducing end of the oligosaccharide and the nitrogen of the amino group of a lysine residue at the carrier protein CRM₁₉₇, wherein the atoms of the backbone are selected from the group consisting of carbon, nitrogen, oxygen and sulphur;
or a pharmaceutically acceptable salt thereof.

5. The immunogenic compound according to claim 4, or a pharmaceutically acceptable salt thereof, wherein
m is 4, n is 2 and R is OH;
m is 4, n is 2 and R is 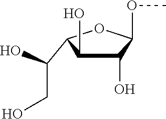
or;
m is 4, n is 3 and R is OH.

6. The immunogenic compound according to claim 4, or a pharmaceutically acceptable salt thereof, wherein m is 4, n is 2 and R is OH.

7. The immunogenic compound according to claim 4, or a pharmaceutically acceptable salt thereof, wherein
-L-T- represents a linker L and a spacer T which together form a bridge having a backbone with a length of 5 to 25 atoms covalently linked together that forms the shortest distance between the oxygen at C1 of the reducing end of the oligosaccharide and the nitrogen of the amino group of a lysine residue at the carrier protein CRM₁₉₇, bearing at most one double bond,
wherein the atoms of the backbone are selected from the group consisting of carbon, nitrogen, oxygen and sulphur, and
wherein the backbone may be substituted with one or more substituents independently selected from oxo, (C₁₋₄)alkyl, fluoro, and (C₁₋₂)alkoxy, and
wherein a part of the backbone optionally may be part of a 4-, 5- or 6-membered ring selected from:

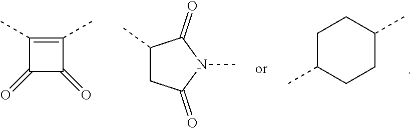

8. The immunogenic compound according to claim 4, or a pharmaceutically acceptable salt thereof, wherein the backbone of the bridge has a length of 8 to 20, atoms covalently linked together that forms the shortest distance between the oxygen at C1 of the reducing end of the oligosaccharide and the nitrogen of the amino group of a lysine residue at the carrier protein CRM₁₉₇.

9. The immunogenic compound according to claim 4, or a pharmaceutically acceptable salt thereof, wherein
L represents
*—(CH₂)ₐ—NH—; wherein a is from 2 to 10;
*—(CH₂CH₂O)_{b}—CH₂CH₂NH—, wherein b is 1, 2 or 3;
*—CH₂CH₂S—CH₂CH₂NH—;
*—(C₂₋₁₀)fluoroalkylene-NH— with fluoroalkylene being a saturated straight chain;
*—(CH₂)_{c}NHC(O)(CH₂)_{c′}—NH—, wherein c and c′ are independently from each other from 2 to 6;
*—(CH₂)_{d}NHC(O)NH(CH₂)_{d′}—NH—, wherein d and d′ are independently from each other from 2 to 6;
*—(CH₂)ₑ—C(O)—NH—(CH₂)_{e′}—NH—; wherein e is from 1 to 10 and e′ is from 2 to 10; or
*—(CH₂)_{f}—O—NH—, wherein f is from 2 to 10; or
L-T represents
*—(CH₂)_{g}—S—R¹, wherein g is from 2 to 10; and
T represents
—C(O)—(CH₂)ₕ—C(O)—, wherein h is from 0 to 10;
—C(O)—CH₂CH₂—(OCH₂CH₂)ⱼ—C(O)—, wherein j is from 1 to 5;
—C(O)—CH₂(CH₂)ₖ—(SCH₂(CH₂)_{k′})_{k″}—C(O)—, wherein k is 0 or 1, k′ is 0 or 1, and k″ is 1, 2, or 3; 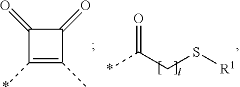
wherein l is 1 or 2; or 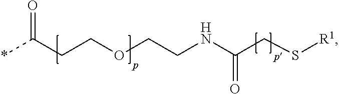
wherein p is from 1 to 4, and p′ is 1 or 2; and
R¹ represents 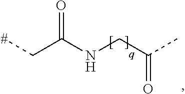
wherein q is 2 or 3; 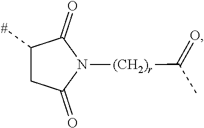
wherein r is 2 or 3; or

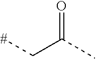

10. The immunogenic compound according to claim 4, or a pharmaceutically acceptable salt thereof, wherein
L represents 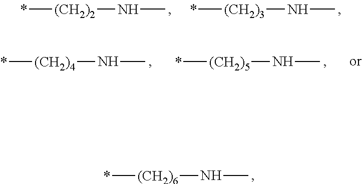
and
T represents

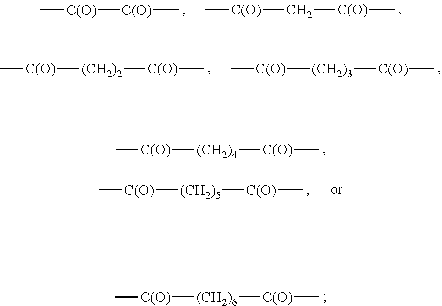

11. The immunogenic compound according to claim 4, or a pharmaceutically acceptable salt thereof, wherein i is from 6 to 15.

12. The immunogenic compound according to claim 4, or a pharmaceutically acceptable salt thereof, selected from the group consisting of the structures of formula (IIa), (IIb) and (IIc): 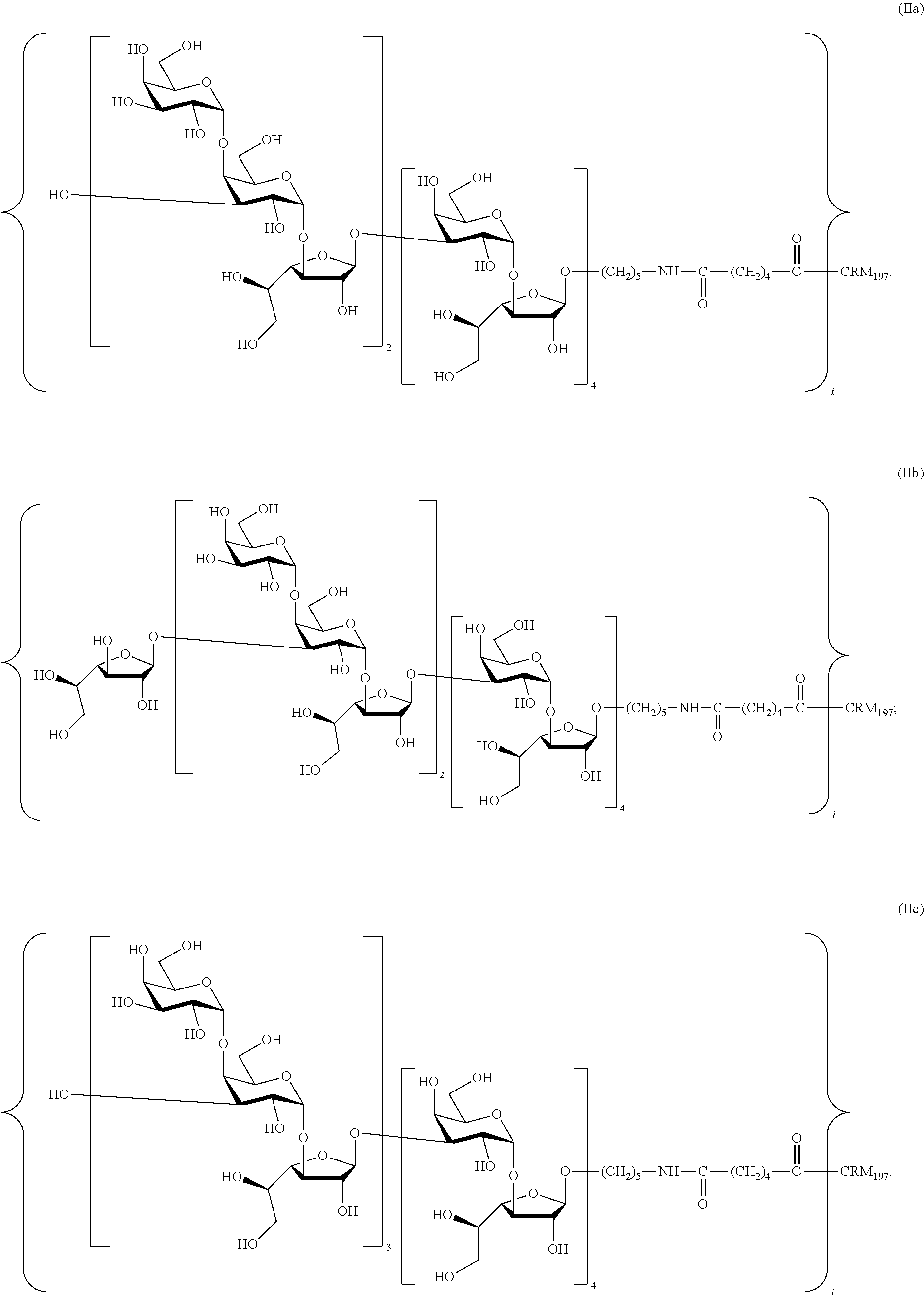
wherein i is from 1 to 28.

13. A pharmaceutical composition comprising, as active principle, an immunogenic compound according to claim 1, or a pharmaceutically acceptable salt thereof, and at least one therapeutically inert excipient.

14. The pharmaceutical composition according to claim 13, further comprising an adjuvant.

15. (canceled)

16. A method for the prevention and/or treatment of a K. pneumoniae infection in a human and/or animal, wherein the method comprises administering to the human and/or animal an effective amount of an immunogenic compound according to claim 1.

17. A multivalent vaccine comprising the immunogenic compound according to claim 1, or a pharmaceutically acceptable salt thereof.

18. A compound having the structure of formula (III): 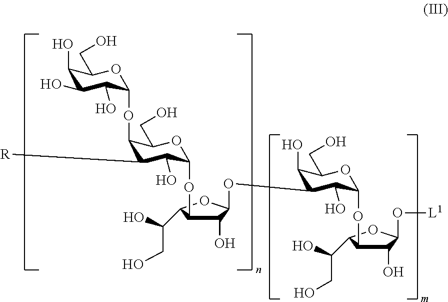
wherein
R is OH, or 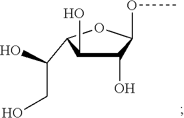
m is 3, 4, 5, 6, 7 or 8; preferably 3, 4, 5 or 6;
n is 1, 2, 3, 4, 5 or 6; preferably 2, 3, or 4;
L¹ represents
*—(C₂₋₁₀)alkylene-NH₂, preferably *—(CH₂)ₐ—NH₂; wherein a is from 2 to 10, more preferably l is 5;
*—(CH₂CH₂O)_{b}—CH₂CH₂NH₂, wherein b is 1, 2 or 3;
*—CH₂CH₂S—CH₂CH₂NH₂;
*—(C₂₋₁₀)fluoroalkylene-NH₂;
*—(CH₂)_{c}NHC(O)(CH₂)_{c′}—NH₂, wherein c and c′ are independently from each other from 2 to 6;
*—(CH₂)_{d}NHC(O)NH(CH₂)_{d′}—NH₂, wherein d and d′ are independently from each other from 2 to 6;
*—(C₁₋₁₀)alkylene-C(O)—NH—(C₂₋₁₀)alkylene-NH₂;
—(C₂₋₁₀)alkylene-O—NH₂; or
*—(C₂₋₁₀)alkylene-SH;
or a pharmaceutically acceptable salt thereof.

19. A compound having the structure of formula (IV): 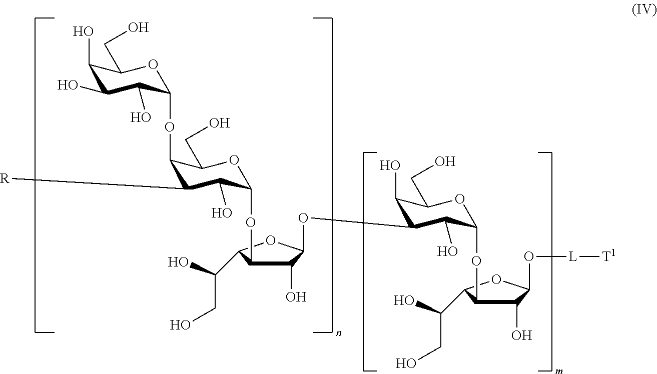
wherein
R is OH, or 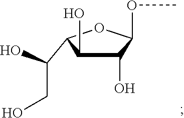
m is 3, 4, 5, 6, 7 or 8; preferably 3, 4, 5 or 6;
n is 1, 2, 3, 4, 5 or 6; preferably 2, 3, or 4;
L represents
*—(C₂₋₁₀)alkylene-NH—, preferably *—(CH₂)ₐ—NH— wherein a is from 2 to 10, more preferably 5;
*—(CH₂CH₂O)_{b}—CH₂CH₂NH—, wherein b is 1, 2 or 3;
*—CH₂CH₂S—CH₂CH₂NH—;
*—(C₂₋₁₀)fluoroalkylene-NH—;
*—(CH₂)_{c}NHC(O)(CH₂)_{c′}—NH—, wherein c and c′ are independently from each other from 2 to 6;
*—(CH₂)_{d}NHC(O)NH(CH₂)_{d′}—NH—, wherein d and d′ are independently from each other from 2 to 6;
*—(C₁₋₁₀)alkylene-C(O)—NH—(C₂₋₁₀)alkylene-NH—; or
*—(C₂₋₁₀)alkylene-O—NH—;
T¹ represents
—C(O)—(C₀₋₁₀)alkylene-C(O)X;
—C(O)—CH₂CH₂—(OCH₂CH₂)ⱼ—C(O)X, wherein j is from 1 to 5;
—C(O)—CH₂(CH₂)ₖ—(SCH₂(CH₂)_{k′})_{k″}—C(O)X, wherein k is 0 or 1, k′ is 0 or 1, and k″ is 1, 2, or 3; 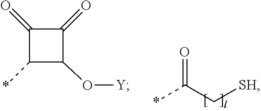
wherein l is 1 or 2; or 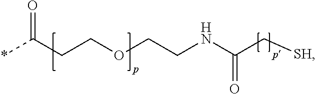
wherein p is from 1 to 4, preferably 1, and
p′ is 1 or 2;
—C(O)X represents —C(O)OH or an activated ester, wherein preferably
X represents 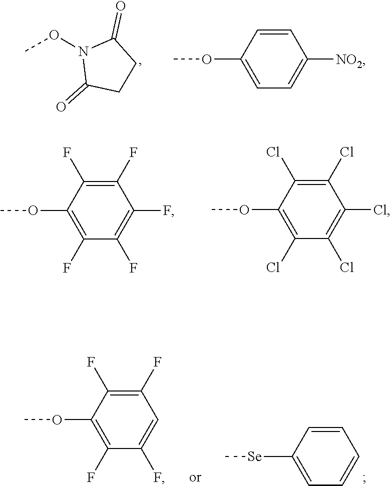
and
Y represents Me, Et, Bu or —(CH₂CH₂O)₃CH₃.

20. The immunogenic compound according to claim 1, or a pharmaceutically acceptable salt thereof, wherein
m is 3, 4, 5 or 6; and
n is 2, 3, or 4.

21. The immunogenic compound according to claim 4, or a pharmaceutically acceptable salt thereof, wherein
m is 3, 4, 5 or 6; and
n is 2, 3, or 4.

22. The immunogenic compound according to claim 4, or a pharmaceutically acceptable salt thereof, wherein the backbone of the bridge has a length of 8 to 16 atoms covalently linked together that forms the shortest distance between the oxygen at C1 of the reducing end of the oligosaccharide and the nitrogen of the amino group of a lysine residue at the carrier protein CRM₁₉₇.

23. The immunogenic compound according to claim 4, or a pharmaceutically acceptable salt thereof, wherein
L represents *—(CH₂)₅—NH—; and
T represents —C(O)—(CH₂)₄—C(O)—.

24. The immunogenic compound according to claim 4, or a pharmaceutically acceptable salt thereof, wherein i is from 6 to 15.

25. A pharmaceutical composition comprising, as active principle, an immunogenic compound according to claim 4, or a pharmaceutically acceptable salt thereof, and at least one therapeutically inert excipient.

26. A pharmaceutical composition comprising, as active principle, an immunogenic compound according to claim 12, or a pharmaceutically acceptable salt thereof, and at least one therapeutically inert excipient.

27. A method for the prevention and/or treatment of a K. pneumoniae infection in a human and/or animal, wherein the method comprises administering to the human and/or animal an effective amount of an immunogenic compound according to claim 4.

28. A method for the prevention and/or treatment of a K. pneumoniae infection in a human and/or animal, wherein the method comprises administering to the human and/or animal an effective amount of an immunogenic compound according to claim 12.

29. A multivalent vaccine comprising the immunogenic compound according to claim 4, or a pharmaceutically acceptable salt thereof.

30. A multivalent vaccine comprising the immunogenic compound according to claim 12, or a pharmaceutically acceptable salt thereof.


SEQUENCE LISTING (6 sequences)

SEQ ID NO: 1 - protein, 535 aa, synthetic construct
GADDVVDSSKSFVMENFSSYHGTKPGYVDSIQKGIQKPKSGTQGNYDDDWKEFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLHDGYAVSWNTVEDSIIRTGFQGESGHDIKITAENTPLPIAGVLLPTIPGKLDVNKSKTHISVNGRKIRMRCRAIDGDVTFCRPKSPVYVGNGVHANLHVAFHRSSSEKIHSNEISSDSIGVLGYQKTVDHTKVNSKLSLFFEIKS

SEQ ID NO: 2 - protein, 535 aa, Corynebacterium diphtheriae
GADDVVDSSKSFVMENFSSYHGTKPGYVDSIQKGIQKPKSGTQGNYDDDWKGFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLHDGYAVSWNTVEDSIIRTGFQGESGHDIKITAENTPLPIAGVLLPTIPGKLDVNKSKTHISVNGRKIRMRCRAIDGDVTFCRPKSPVYVGNGVHANLHVAFHRSSSEKIHSNEISSDSIGVLGYQKTVDHTKVNSKLSLFFEIKS

SEQ ID NO: 3 - protein, 1314 aa, Clostridium tetani
PITINNFRYSDPVNNDTIIMMEPPYCKGLDIYYKAFKITDRIWIVPERYEFGTKPEDFNPPSSLIEGASEYYDPNYLRTDSDKDRFLQTMVKLFNRIKNNVAGEALLDKIINAIPYLGNSYSLLDKFDTNSNSVSFNLLEQDPSGATTKSAMLTNLIIFGPGPVLNKNEVRGIVLRVDNKNYFPCRDGFGSIMQMAFCPEYVPTFDNVIENITSLTIGKSKYFQDPALLLMHELIHVLHGLYGMQVSSHEIIPSKQEIYMQHTYPISAEELFTFGGQDANLISIDIKNDLYEKTLNDYKAIANKLSQVTSCNDPNIDIDSYKQIYQQKYQFDKDSNGQYIVNEDKFQILYNSIMYGFTEIELGKKFNIKTRLSYFSMNHDPVKIPNLLDDTIYNDTEGFNIESKDLKSEYKGQNMRVNTNAFRNVDGSGLVSKLIGLCKKIIPPTNIRENLYNRTASLTDLGGELCIKIKNEDLTFIAEKNSFSEEPFQDEIVSYNTKNKPLNFNYSLDKIIVDYNLQSKITLPNDRTTPVTKGIPYAPEYKSNAASTIEIHNIDDNTIYQYLYAQKSPTTLQRITMTNSVDDALINSTKIYSYFPSVISKVNQGAQGILFLQWVRDIIDDFTNESSQKTTIDKISDVSTIVPYIGPALNIVKQGYEGNFIGALETTGVVLLLEYIPEITLPVIAALSIAESSTQKEKIIKTIDNFLEKRYEKWIEVYKLVKAKWLGTVNTQFQKRSYQMYRSLEYQVDAIKKIIDYEYKIYSGPDKEQIADEINNLKNKLEEKANKAMININIFMRESSRSFLVNQMINEAKKQLLEFDTQSKNILMQYIKANSKFIGITELKKLESKINKVFSTPIPFSYSKNLDCWVDNEEDIDVILKKSTILNLDINNDIISDISGFNSSVITYPDAQLVPGINGKAIHLVNNESSEVIVHKAMDIEYNDMFNNFTVSFWLRVPKVSASHLEQYGTNEYSIISSMKKHSLSIGSGWSVSLKGNNLIWTLKDSAGEVRQITFRDLPDKFNAYLANKWVFITITNDRLSSANLYINGVLMGSAEITGLGAIREDNNITLKLDRCNNNNQYVSIDKFRIFCKALNPKEIEKLYTSYLSITFLRDFWGNPLRYDTEYYLIPVASSSKDVQLKNITDYMYLTNAPSYTNGKLNIYYRRLYNGLKFIIKRYTPNNEIDSFVKSGDFIKLYVSYNNNEHIVGYPKDGNAFNNLDRILRVGYNAPGIPLYKKMEAVKLRDLKTYSVQLKLYDDKNASLGLVGTHNGQIGNDPNRDILIASNWYFNHLKDKILGCDWYFVPTDEGWTND

SEQ ID NO: 4 - protein, 103 aa, Vibrio cholerae
TPQNITDLCAEYHNTQIYTLNDKIFSYTESLAGKREMAIITFKNGAIFQVEVPGSQHIDSQKKAIERMKDTLRIAYLTEAKVEKLCVWNNKTPHAIAAISMAN

SEQ ID NO: 5 - protein, 272 aa, Neisseria meningitidis
MKKTVFTCAMIALTGTAAAAQELQTANEFTVHTDLSSISSTRAFLKEKHKAAKHISVRADIPFDANQGIRLEAGFGRSKKNIINLETDENKLGKTKNVKLPTGVPENRIDLYTGYTYTQTLSDSLNFRVGAGLGFESSKDSIKTTKHTLHSSRQSWLAKVHADLLSQLGNGWYINPWSEVKFDLNSRYKLNTGVTNLKKDINQKTNGWGFGLGANIGKKLGESASIEAGPFYKQRTYKESGEFSVTTKSGDVSLTIPKTSIREYGLRVGIKF

SEQ ID NO: 6 - protein, 132 aa, Escherichia virus Qbeta
AKLETVTLGNIGKDGKQTLVLNPRGVNPTNGVASLSQAGAVPALEKRVTVSVSQPSRNRKNYKVQVKIQNPTACTANGSCDPSVTRQAYADVTFSFTQYSTDEERAFVRTELAALLASPLLIDAIDQLNPAY